(19) United States
(12) Patent Application Publication
(10) Pub. No.: US 2026/0083655 A1
(43) Pub. Date: Mar. 26, 2026

(54) COMPOSITIONS COMPRISING SOPHOROLIPIDS AND LACTIC ACID

(71) Applicant: Evonik Operations GmbH, Essen (DE)

(72) Inventors: Stefan Neubauer, Koeln (DE); Stefan Julian Liebig, Duesseldorf (DE); Alina Muss, Essen (DE); Nadine Truchan, Duesseldorf (DE)

(73) Assignee: Evonik Operations GmbH, Essen (DE)

(21) Appl. No.: 19/113,041

(22) PCT Filed: Sep. 13, 2023
(86) PCT No.: PCT/EP2023/075089

§ 371 Date: Mar. 19, 2025

(30) Foreign Application Priority Data
Sep. 26, 2022 (EP) 22197747.3

(51) Int. Cl. A61K 8/60; A61K 8/34; A61K 8/365; A61K 8/73; A61Q 19/00

(52) U.S. Cl. CPC A61K 8/602; A61K 8/34; A61K 8/365; A61K 8/731; A61Q 19/00; A61K 2800/10

(57) ABSTRACT

The present invention relates to compositions containing at least one sophorolipid and lactic acid or its salt, in certain concentrations. The compositions can contain lactone or acid type sophorolipids, monohydric alcohols, and water. The present invention also relates to methods of utilizing the compositions of the invention, such as for cleaning a surface or moisturizing skin.

### FIELD OF THE INVENTION

The invention relates to compositions comprising sophorolipids and lactic acid or its salt in certain concentrations.

### PRIOR ART

US2022064577 discloses cleaning compositions, comprising at least one pH adjusting agent and at least one biosurfactant selected from the group consisting of glycolipids, lipopeptides, and mixtures thereof, wherein the composition has a pH of from about 1 to about 14.

A disadvantage of the disclosed system is its very low concentrations of biosurfactants and therefore, included in formulations has no real influence on the sustainability profile. Another disadvantage is the usage of a racemic solution of lactic acid and therefore, it cannot be assured that the D-lactic acid is excluded from the solution, which should be avoided. It is not a highly toxic compound but identified as a significant marker and toxic metabolite that can cause health problems (Pohanka M. D-Lactic Acid as a Metabolite: Toxicology, Diagnosis, and Detection. Biomed Res Int. 2020 Jun. 17; 2020).

IN202011005229 discloses different cleansing bodybath formulations for pets, that contain Rhamnolipids as surfactant. Among others lactic acid is mentioned as pH-adjuster. Described is as well that the bodybath itself shows antimicrobial properties. One disadvantage is that the biosurfactants' antimicrobial activity could potentially disbalance the skin's microbiome. Another disadvantage is the usage of a racemic solution of lactic acid and therefore, it cannot be assured that the D-lactic acid is excluded from the solution, which should be avoided. It is not a highly toxic compound but identified as a significant marker and toxic metabolite that can cause health problems (Pohanka M. D-Lactic Acid as a Metabolite: Toxicology, Diagnosis, and Detection. Biomed Res Int. 2020 Jun. 17; 2020).

Silveira, V.A.I., Kobayashi, R.K.T., de Oliveira Junior, A. G. et al. evaluated the antibacterial effect of sophorolipids in combination with lactic acid in an ethanol-based solution against certain bacteria (Braz. J. Microbiol. 2021, 52, 1769-1778). As disadvantage the addition of ethanol can be listed, as its property as penetration enhancer can lead to skin irritations as well as dry skin. Another disadvantage is that ethanol can influence the viscosity and be responsible of a low viscosity of the formulation. Another disadvantage in regards of the cleansing power is the identified lactonic C18:1 diacetylated form, which is lower than the respective acid form. This patent lists the ratio of lactic acid and sophorolipids and uses 10 times more lactic acid than sophorolipids, which can be seen as a disadvantage as well, as it can influence the pKs value of the formulation. Another disadvantage is the usage of a racemic solution of lactic acid and therefore, it cannot be assured that the D-lactic acid is excluded from the solution, which should be avoided. It is not a highly toxic compound but identified as a significant marker and toxic metabolite that can cause health problems (Pohanka M. D-Lactic Acid as a Metabolite: Toxicology, Diagnosis, and Detection. Biomed Res Int. 2020 Jun. 17; 2020).

The group of I.M. Banat (FEMS Microbiology Letters. 2016, 363, fnv224) showed that biosurfactants can have antibacterial properties against certain gram-positive and gram-negative bacteria. As example a combination of 1% sophorolipid and 0.8% lactic acid is listed. One disadvantage of this application is the relatively high ratio of lactic acid to biosurfactants, which might lead to an increased irritation potential on the skin. These high concentrations as well as the antimicrobial activity of the biosurfactants could potentially disbalance the skin's microbiome and lead to inflammatory skin conditions. Another disadvantage is the usage of a racemic solution of lactic acid and therefore, it cannot be assured that the D-lactic acid is excluded from the solution, which should be avoided. It is not a highly toxic compound but identified as a significant marker and toxic metabolite that can cause health problems (Pohanka M. D-Lactic Acid as a Metabolite: Toxicology, Diagnosis, and Detection. Biomed Res Int. 2020 Jun. 17; 2020).

It is an object of the invention to provide compositions with outstanding cleaning properties, especially of skin surfaces.

### DESCRIPTION OF THE INVENTION

It was found that, surprisingly, that a combination of sophorolipids and lactic acid, solves the problem of the instant invention.

The present invention therefore provides compositions comprising sophorolipids and lactic acid or its salt in certain concentrations and their use.

One advantage of the compositions according to the invention is their decreased foaming, which is desirable in for example eye make-up removal applications and advantageous especially in cleaning process conducted by machines to avoid leakage of excess foam.

A further advantage is the reduced necessity for microbial active preservatives.

A further advantage of the composition according to the invention is its low viscosity and therefore simple processability in any desired aqueous surface-active system.

A further advantage of the compositions according to the invention is their good skin and hair cleansing properties.

A further advantage of the compositions according to the invention is their good make-up removal property.

A further advantage of the compositions according to the invention is their good lipstick removal property.

A further advantage of the compositions according to the invention is their good eye make-up removal property.

A further advantage of the compositions according to the invention is their very good solubilizing efficacy for essential oils at low usage levels.

A further advantage of the composition according to the invention is their mildness and good physiological compatibility.

A further advantage of the compositions according to the invention is their good skin feel during and after washing.

A further advantage of the compositions according to the invention is their good rinseability compared to other surface-active compositions.

A further advantage of the compositions according to the invention is that they leave a smooth and soft skin feel after washing.

A further advantage of the mixture compositions according to the invention is that they can be synthesized free from petrochemical-based raw materials.

A further advantage of the mixture compositions according to the invention is that they can be synthesized free from critical raw materials such as tropical oils.

A further advantage of the mixture compositions according to the invention is their outstanding microbiological stability.

A further advantage is that L-lactic acid enantiomer can be comprised, which can be produced solely obtained by fermentation and therefore is a sustainably and natural raw material.

A further advantage is that the acidic sophorolipid shows improved make up removal performance when preserved with lactic acid.

A further advantage of the compositions according to the invention is their outstanding properties as emulsifier, especially when the sophorolipids comprise a high level of lactonic form.

A further advantage of the compositions according to the invention is their outstanding properties as solubilizer, especially when the sophorolipids comprise a high level of lactonic form.

A further advantage of the compositions according to the invention is their activity against acne, especially when the sophorolipids comprise a high level of lactonic form.

A further advantage of the compositions according to the invention is their positive influence on microbiomes, for example on the skin or on textile surfaces, especially when the sophorolipids comprise a high level of lactonic form.

The instant invention encompasses a composition comprising
a) at least one sophorolipid
b) lactic acid and/or a salt thereof,
characterized in that
a) is comprised in an amount of from 1.2 wt.-% to 70.0 wt.-% and
b) is comprised in an amount of from 0.08 wt.-% to 10.0 wt.-%,
wherein the weight percentages refer to the total composition.

Unless stated otherwise, percentages are data in percent by weight.

Wherever measurement values are stated hereinbelow, then, unless stated otherwise, these have been determined at a temperature of 25° C. and a pressure of 1013 mbar.

The “pH” in connection with the present invention—unless stated otherwise—is defined as the value which is measured for the relevant composition at 25° C. after stirring for five minutes using a pH electrode calibrated in accordance with ISO 4316 (1977).

In the context of the present invention, the term “sophorolipids” preferably is understood as meaning compounds of the general formulae (la) and (Ib) and salts thereof

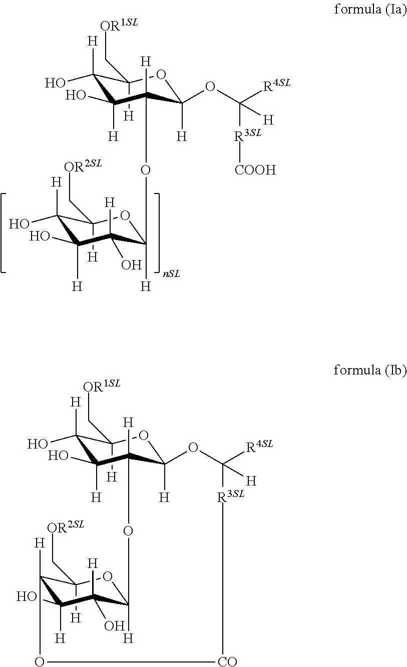

where
R^{1SL}=H or CO—CH₃,
R^{2SL}=H or CO—CH₃,
R^{3SL}=a divalent organic moiety which comprises 6 to 32 carbon atoms and which is unsubstituted or substituted by hydroxyl functions, is unbranched and optionally comprises one to three double or triple bonds,
R^{4SL}=H, CH₃ or a monovalent organic radical which comprises 2 to 10 carbon atoms and which is unsubstituted or substituted by hydroxyl functions, which is unbranched and which optionally comprises one to three double or triple bonds, and
nSL=1 or 0.

Sophorolipids may be used in accordance with the invention in their acid form or their lactone form.

Preferred compositions according to the instant invention are characterized in that the component a) has a content of lactone type sophorolipids of more than 50 wt-%, preferably more than 80 wt.-%, more preferably more than 95 wt.-%, wherein the weight percentages refer to the total component a).

To determine the content of sophorolipids in the acid or lactone form in a composition, refer to EP1411111B1, page 8, paragraph [0053].

Alternatively preferred compositions according to the instant invention are characterized in that the component a) has a content of acid type sophorolipids of from 55 wt.-% to 99 wt.-%, preferably 58 wt.-% to 98 wt.-%, more preferably 82 wt.-% to 95 wt.-%,

A preferred composition according to the instant invention is characterized in that, the total content of monohydric alcohols chosen from methanol, ethanol, propanol, isopropanol and butanol is in the range of from 0.0 wt.-% to 10.0 wt.-%, preferably from 0.0 wt.-% to 2.0 wt.-%, wherein the weight percentages refer to the total composition.

A preferred composition according to the instant invention is characterized in that, it comprises water, preferably in an amount of from 25.0 wt.-% to 95.0 wt.-%, wherein the weight percentages refer to the total composition.

A preferred composition according to the instant invention is characterized in that the weight ratio of component a) to component b) is from 5:1 to 30:1, preferably from 10:1 to 20:1.

The lactic acid in component b) can be comprised as a free acid or in its salt form.

The counter ion in the lactic acid's salt form is preferably chosen from the group of sodium and ammonium, preferably sodium.

Lactic acid can be included in the form of both enantiomers, D- or L-.

Mixtures and also the racemate can be used, of course.

A preferred composition according to the instant invention is characterized in that in component b) the L-enantiomer content is higher than 51.0 wt-%, preferably higher than 80.0 wt.-%, more preferably higher than 95 wt.-%,
wherein the weight percentages refer to the total component b).

The composition according to the instant invention preferably has a pH of 3.5 to 11.0, preferably of 4.0 to 9.0, particularly preferably of 4.5 to 7.0.

A preferred composition according to the instant invention is characterized in that
a) is comprised in an amount of from 30.0 wt.-% to 55.0 wt.-%,
b) is comprised in an amount of from 1.0 wt.-% to 5.0 wt.-%, and
water is comprised in an amount of from 30.0 wt.-% to 69.0 wt.-%,
wherein the weight percentages refer to the sum of a) and b) and water.

A preferred composition according to the instant invention is a concentrated composition, which can serve as a stock composition, for example as base ingredient for the preparation of formulations.

Therefore, a preferred composition according to any of the preceding claims, characterized in that
a) is comprised in an amount of from 30.0 wt.-% to 55.0 wt.-%,
b) is comprised in an amount of from 1.0 wt.-% to 5.0 wt.-%, and
water is comprised in an amount of from 40.0 wt.-% to 69.0 wt.-%,
wherein the weight percentages refer to the total composition.

Of course, a complex formulation for a specific purpose produced by combining the concentrated stock composition of the instant invention with further ingredients is also part of the instant invention.

Thus, a preferred composition according to the instant invention, preferably being a cosmetic, pharmaceutical or household care formulation, is characterized in that
a) is comprised in an amount of from 1.2 wt.-% to 10.0 wt.-%, and
b) is comprised in an amount of from 0.08 wt.-% to 3.0 wt.-%,
wherein the weight percentages refer to the total composition,
is encompassed by the instant invention.

The composition according to the instant invention can be formulated as household care composition; preferably in this instance the following applies:

The formulations according to the instant invention can further comprise one or more auxiliary agents selected from the group consisting of bleaching systems, hydrotropes, polymers, which may be synthetic, biopolymers, anti-redeposition aids, fiber protection agents, soil release agents, dye transfer inhibitors, fabric hueing agents, opacifiers, blueing dyes, enzyme stabilizing agents, solvents, viscosity modifiers, preservatives, pH-regulators and salts like NaCl and Na2SO4.

The composition according to the instant invention can be formulated as a cosmetic or pharmaceutical composition; preferably in this instance the following applies:

The compositions according to the invention can further comprise at least one additional component selected from the group of
emollients,
emulsifiers,
thickeners/viscosity regulators/stabilizers,
UV light protection filters,
antioxidants,
hydrotropes,
solids and fillers,
film formers,
pearlescence additives,
deodorant and antiperspirant active ingredients,
insect repellents,
self-tanning agents,
preservatives,
conditioning agents,
perfumes,
dyes,
odour absorbers,
superfatting agents,
other solvents.

Substances which can be used as exemplary representatives of the individual groups are known to those skilled in the art and can be found for example in German application DE 102008001788.4. This patent application is hereby incorporated as reference and thus forms part of the disclosure. As regards further optional components and the amounts used of these components, reference is made expressly to the relevant handbooks known to those skilled in the art, for example K.

Schrader, “Grundlagen und Rezepturen der Kosmetika [Cosmetics—fundamentals and formulations]”, 2nd edition, pages 329 to 341, HOthig Buch Verlag Heidelberg.

The amounts of the particular additives are determined by the intended use.

Typical boundary formulations for the respective applications are known prior art and are contained for example in the brochures of the manufacturers of the particular base and active ingredients.

These existing formulations can generally be adopted unchanged. However, if required, for adjustment and optimization, the desired modifications can be undertaken by simple tests without complication.

A preferred composition according to the instant inventions comprises a further biosurfactant, preferably selected from the group of rhamnolipids, glucolipids, cellulose lipids, mannosylerythritol lipids and trehalose lipids, preferably rhamnolipids and glucolipids, most preferably rhamnolipids.

Within the context of the present invention, “biosurfactants” are understood as meaning all glycolipids produced by fermentation. The term “biosurfactant” also covers glycolipids that are chemically or enzymatically modified after fermentation, as long as structurally a glycolipid remains. Raw materials for producing the biosurfactants that can be used are carbohydrates, in particular sugars such as e.g. glucose and/or lipophilic carbon sources such as fats, oils, partial glycerides, fatty acids, fatty alcohols, long-chain saturated or unsaturated hydrocarbons.

In the context of the present invention, the terms “surfactant” is understood to mean organic substances having interface-active properties that have the ability to reduce the surface tension of water at 20° C. and at a concentration of 0.5% by weight based on the overall composition to below 45 mN/m. Surface tension is determined by the Du Noüy ring method at 20° C.

The term “rhamnolipids” in the context of the present invention preferably is understood to mean particularly compounds of the general formula (I) and salts thereof,

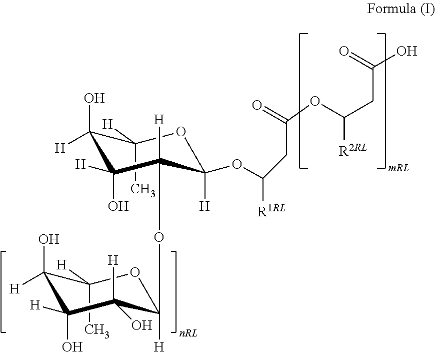

where
mRL=2, 1 or 0,
nRL=1 or 0,
R^{1RL} and R^{2RL}=mutually independently, identical or different, organic residues having 2 to 24, preferably 5 to 13 carbon atoms, in particular optionally branched, optionally substituted, particularly hydroxy-substituted, optionally unsaturated, in particular optionally mono-, bi- or tri-unsaturated alkyl residues, preferably those selected from the group consisting of pentenyl, heptenyl, nonenyl, undecenyl and tridecenyl and (CH₂)ₒ—CH₃ where o=1 to 23, preferably 4 to 12. If nRL=1, the glycosidic bond between the two rhamnose units is preferably in the α-configuration. The optically active carbon atoms of the fatty acids are preferably present as R-enantiomers (e.g. (R)-3-{(R)-3-[2-O-(α-L-rhamnopyranosyl)-α-L-rhamnopyranosyl]oxydecanoyl}oxydecanoate).

The term “di-rhamnolipid” in the context of the present invention is understood to mean compounds of the general formula (I) or salts thereof, where nRL=1.

The term “mono-rhamnolipid” in the context of the present invention is understood to mean compounds of the general formula (I) or salts thereof, where nRL=0.

Distinct rhamnolipids are abbreviated according to the following nomenclature: “diRL-CXCY” are understood to mean di-rhamnolipids of the general formula (I), in which one of the residues R^{1RL} and R^{2RL}=(CH₂)ₒ—CH₃ where o=X-4 and the remaining residue R¹ or R²=(CH₂)ₒ—CH₃ where o=Y-4.

“monoRL-CXCY” are understood to mean mono-rhamnolipids of the general formula (I), in which one of the residues R^{1RL} and R^{2RL}=(CH₂)ₒ—CH₃ where o=X-4 and the remaining residue R^{1RL} or R^{2RL}=(CH₂)ₒ—CH₃ where o=Y-4.

The nomenclature used therefore does not distinguish between “CXCY” and “CYCX”.

For rhamnolipids where mRL=0, monoRL-CX or diRL-CX is used accordingly.

If one of the abovementioned indices X and/or Y is provided with “:Z”, this signifies that the respective residue R^{1RL} and/or R^{2R}L is equal to an unbranched, unsubstituted hydrocarbon residue having X-3 or Y-3 carbon atoms having Z double bonds.

Methods for preparing the relevant rhamnolipids are disclosed, for example, in EP2786743 and EP2787065.

Rhamnolipids applicable in the context of the instant invention can also be produced by fermentation of Pseudomonas, especially Pseudomonas aeruginosa, which are preferably non genetically modified cells, a technology already disclosed in the eighties, as documented e.g. in EP0282942 and DE4127908. Rhamnolipids produced in Pseudomonas aeruginosa cells which have been improved for higher rhamnolipid titres by genetical modification can also be used in the context of the instant invention; such cells have for example been disclosed by Lei et al. in Biotechnol Lett. 2020 June; 42(6):997-1002.

Rhamnolipids produced by Pseudomonas aeruginosa are commercially available from Jeneil Biotech Inc., e.g. under the tradename Zonix, from Logos Technologies (technology acquired by Stepan), e.g. under the tradename NatSurFact, from Biotensidion GmbH, e.g. under the tradename Rhapynal, from AGAE technologies, e.g. under the name R90, R95, R95Md, R95Dd, from Locus Bio-Energy Solutions and from Shanghai Yusheng Industry Co. Ltd., e.g. under the tradename Bio-201 Glycolipids.

The present invention provides a composition preferably comprising as further biosurfactant rhamnolipids, characterized in that it comprises
51% by weight to 100% by weight, preferably 60% by weight to 95% by weight, particularly preferably 80% by weight to 90% by weight, of mono-rhamnolipids, especially those of formula (I) with nRL=0,
where the percentages by weight refer to the sum of all of the rhamnolipids present.

The present invention further provides a composition preferably comprising as further biosurfactant rhamnolipids, characterized in that it comprises
51% by weight to 95% by weight, preferably 55% by weight to 80% by weight, particularly preferably 60% by weight to 70% by weight, of diRL-C10C10,
where the percentages by weight refer to the sum of all of the rhamnolipids present.

A preferred composition according to the invention is characterized in that the composition comprises as further biosurfactant rhamnolipids as described above with a content of
0.5% by weight to 15% by weight, preferably 3% by weight to 12% by weight, particularly preferably 5% by weight to 10% by weight, of diRL-C10C12:1,
where the percentages by weight refer to the sum of all of the rhamnolipids present.

A further preferred composition according to the invention is characterized in that the composition comprises as further biosurfactant rhamnolipids as described above with a content of 0.5 to 25% by weight, preferably 3% by weight to 15% by weight, particularly preferably 5% by weight to 12% by weight, of diRL-C10C12, where the percentages by weight refer to the sum of all of the rhamnolipids present.

A further preferred composition according to the invention is characterized in that the composition comprises as further biosurfactant rhamnolipids as described above with a content of 0.1% by weight to 25% by weight, preferably 2% by weight to 10% by weight, particularly preferably 4% by weight to 8% by weight, of diRL-C8C10,
where the percentages by weight refer to the sum total of all rhamnolipids present.

An even further preferred composition according to the invention is characterized in that the composition comprises as further biosurfactant rhamnolipids as described above with a content of
0.1% by weight to 5% by weight, preferably 0.5% by weight to 3% by weight, particularly preferably
0.5% by weight to 2% by weight, of monoRL-C8C10 and/or, preferably and
0.1% by weight to 5% by weight, preferably 0.5% by weight to 3% by weight, particularly preferably
0.5% by weight to 2% by weight, of monoRL-C10C10,
where the percentages by weight refer to the sum of all of the rhamnolipids present.

The present invention further provides a composition alterntively preferably comprising as further biosurfactant rhamnolipids, characterized in that it comprises
10% by weight to 50% by weight, preferably 20% by weight to 40% by weight, particularly preferably 25% by weight to 35% by weight, of monoRL-C10C10 and
where the percentages by weight refer to the sum of all of the rhamnolipids present.

The alternatively preferred composition according to the invention is preferably characterized in that the composition comprises as further biosurfactant rhamnolipids as described above with a content of
10% by weight to 30% by weight, preferably 12% by weight to 25% by weight, particularly preferably
15% by weight to 20% by weight, of diRL-C10C10,
where the percentages by weight refer to the sum of all of the rhamnolipids present.

The alternatively preferred composition according to the invention is preferably characterized in that the composition comprises as further biosurfactant rhamnolipids as described above with a content of
10% by weight to 30% by weight, preferably 12% by weight to 25% by weight, particularly preferably
15% by weight to 20% by weight, of monoRL-C8C10,
where the percentages by weight refer to the sum of all of the rhamnolipids present.

The alternatively preferred composition according to the invention is preferably characterized in that the composition comprises as further biosurfactant rhamnolipids as described above with a content of
3% by weight to 25% by weight, preferably 5% by weight to 20% by weight, particularly preferably
10% by weight to 15% by weight, of monoRL-C10C12:1,
where the percentages by weight refer to the sum total of all rhamnolipids present.

The alternatively preferred composition according to the invention is preferably characterized in that the composition comprises as further biosurfactant rhamnolipids as described above with a content of
1% by weight to 15% by weight, preferably 2% by weight to 10% by weight, particularly preferably 3% by weight to 8% by weight, of diRL-C10C12,
where the percentages by weight refer to the sum of all of the rhamnolipids present.

Methods for preparing the relevant rhamnolipids are disclosed, for example, in EP2786743 and EP2787065.

Rhamnolipids applicable in the context of the instant invention can also be produced by fermentation of Pseudomonas, especially Pseudomonas aeruginosa, which are preferably non genetically modified cells, a technology already disclosed in the eighties, as documented e.g. in EP0282942 and DE4127908. Rhamnolipids produced in Pseudomonas aeruginosa cells which have been improved for higher rhamnolipid titres by genetical modification can also be used in the context of the instant invention; such cells have for example been disclosed by Lei et al. in Biotechnol Lett. 2020 June; 42(6):997-1002.

Rhamnolipids produced by Pseudomonas aeruginosa are commercially available from Jeneil Biotech Inc., e.g. under the tradename Zonix, from Logos Technologies (technology acquired by Stepan), e.g. under the tradename NatSurFact, from Biotensidion GmbH, e.g. under the tradename Rhapynal, from AGAE technologies, e.g. under the name R90, R95, R95Md, R95Dd, from Locus Bio-Energy Solutions and from Shanghai Yusheng Industry Co. Ltd., e.g. under the tradename Bio-201 Glycolipids.

The compositions according to the instant invention can be used in different fields of applications, for example in various cleaning applications.

In this context preferred composition according to the instant inventions are formulations, preferably cosmetic, laundry or dishwashing formulations.

A further aspect of the instant invention is the use of a composition according to the instant invention for cleaning of a surface, preferably selected from skin, a dish surface or a textile's surface, most preferably skin.

A further aspect of the instant invention is the use of a composition according to the instant invention for moisturizing human skin.

### BRIEF DESCRIPTION OF THE FIGURES

FIG. 1 illustrates the foam volume over time for test solutions

The examples adduced hereinafter describe the present invention by way of example, without any intention that the invention, the scope of application of which is apparent from the entirety of the description and the claims, be restricted to the embodiments specified in the examples.

### EXAMPLES

### Example 1: Preparation of a Composition According to the Invention

### Preparation of Sophorolipids

The crude product was prepared by means of fermentation with the yeast Candida bombicola on the basis of the substrates glucose, sunflower oil, rapeseed oil or olive oil (comprising primarily oleic acid as fatty acid fraction). The growth medium contained the following constituents: 10 g/L glucose (D-(+)-glucose*1H₂O), 7.5 g/L YNB (Yeast Nitrogen Base), 2 g/L yeast extract 1.1 L of the medium were autoclaved in a fermenter with a capacity of 2 L and were seeded with an exponential-phase preculture from the same medium. The temperature was set to 30° C. The p(O₂) was maintained at 30% relative saturation by admission of air via the stirrer speed, but the stirrer speed was never lower than 200 rpm. During the biomass formation phase, the pH dropped to 3.5 and was maintained at this level by addition of NaOH. After the end of the biomass formation phase (consumption of the glucose present, marked by the rise in p(02) or drop in p(C02)), the product formation phase was initiated by addition of 150 g of the corresponding oil, 200 mL of a 750 g/L glucose solution, and 10 mL of a 150 g/L yeast extract solution. The end of the product formation phase was marked by a renewed rise in p(O₂). After fermentation, the batch was autoclaved, with the crude product phase depositing as sediment. The crude product phase was washed with water and hexane, accordingly. The product phase was subsequently extracted with ethyl acetate and then the solvent was removed under reduced pressure. This gave a largely water-free product, corresponding to the dry mass of the invention.

Analysis by means of HPLC-MS and NMR showed that the product is composed largely of the diacetylated sophorolipid lactone form with glycosidically linked fatty acid (main constituents: sophorolipid lactone 65-80% by weight referring to all sophorolipids, fatty acid 1-16% by weight referring to the total composition, glycerol 1-3% by weight referring to the total composition). This component served as sophorolipids in composition C and D (high lactone form).

A 60% by weight aqueous suspension of the sophorolipid was admixed with 0.5% by weight of solid NaOH pellets. The mixture was then stirred at a temperature of 50° C. for 30 minutes in order to give, by hydrolysis, the deacetylated acid form of the sophorolipid.

The batch was then adjusted to a pH of 3 by addition of HCl, and the product was extracted with ethyl acetate. Removal of ethyl acetate gave a residue which can be ground to a powder and has a water solubility of >50% by weight.

This component served as sophorolipids in composition A and B (high acidic form) with a content of the acidic form or more than 55 wt.-% referring to all sophorolipids.

### Preparation of Composition A

Sophorolipids with high acidic content were dissolved in water and L-(+)-lactic acid was added under stirring, to receive the final product with a L-(+)-lactic acid concentration of 3% and a sophorolipid concentration of 45%. The pH value was adjusted to 5.

### Preparation of Non-Inventive Composition B

Sophorolipids with high acidic content were dissolved in water and sodium benzoate was added to the mixture under stirring, to receive the final product with a sodium benzoate concentration of 1% and a sophorolipid concentration of 45%. The pH value was adjusted to 5.

### Preparation of Non-Inventive Composition C

Sophorolipids with a higher lactonic form were dissolved in water and sodium benzoate was added to the mixture under stirring, to receive the final product with a sodium benzoate concentration of 1% and a sophorolipid concentration of 45%. The pH value was adjusted to 5.

### Preparation of Inventive Composition D

Sophorolipids with a higher lactonic form were dissolved in water and L-(+)-lactic acid was added to the mixture under stirring, to receive the final product with a L-(+)-lactic acid concentration of 3% and a sophorolipid concentration of 45%. The pH value was adjusted to 5.

### Example 2: Make-Up Removal Performance

The cleansing properties were determined by removing make-up from PMMA plates using a scrub abrasion and washability device. This method is designed to test the cleansing efficacy of aqueous surfactant solutions such as micellar waters or facial toners and can be used for make-up types that are able to dry completely on the described plates.

### Procedure:

At first the colour value of the PMMA plate itself is measured at three defined positions, using the respective positioning devices. As a second step the make-up is applied with a bar applicator. After one hour of drying the colour value of the coated PMMA plate is measured at the three exact same positions as before. The plate is fixed at the correct position, 2.5 g of the respective test solution, which are prepared by diluting each test substances with water to 3 wt %. Each one is applied onto a cotton pad and the cotton pad is attached to the sponge of the sliding carriage. After positioning of the sliding carriage, the removal experiment starts. The cycles are being set, whereas 1 cycle equals 1 movement forth and back. Speed: 10 cycles per minute, 1 cycle covers 160 mm. After one hour of drying the colour value on the plate is measured at the 3 positions as before and the make-up removal properties determined in & by using the ΔE values (Δ empty plate/coated plate resp. Δ coated plate/cleaned plate), using rule of three.

### Preparation of Test Solution:

All test substances of table 1 were diluted with water to a 3 wt % solution and adjusted to a pH of 7.

TABLE 1

| | | Make-Up- |
|---|---|---|
| | | removal |
| | | (%) |
| Composition A | According to the invention | 90.62 |
| Composition B | Not according to the invention | 88.28 |
| Water | Not according to the invention | 3.96 |
| Polyglyceryl-6 Caprylate | Not according to the invention | 57.99 |
| (and) Polyglyceryl-4 Caprate | | |
| Composition C | Not according to the invention | 79.00 |
| Composition D | According to the invention | 87.000 |

The results in Table 1 show that Composition A has a better make-up removal performance than composition B, composition D being better than C, with composition A comprising sophorolipids with a high acidic content outperforming all of them; water and the benchmark are doing worst.

### Example 3: Evaluation of Foaming Properties Using the SITA Foam Tester

Foamability of surfactants and surfactant-based cleansing products is an important consumer-perceived attribute. Consumers associate fast flash foaming and high foam volumes with efficacy and high quality. Both parameters can be determined using the “SITA foam tester R-2000” measuring device from SITA Messtechnik GmbH. In this device, foam is generated by introducing air into a defined volume of a surfactant solution through a special rotor. The total volume of liquid and resulting foam is measured over time by means of a computer-controlled sensing technique. Using this method, Composition A containing sophorolipid and lactic acid was evaluated for its foamability in comparison to Composition B containing sophorolipid and sodium benzoate.

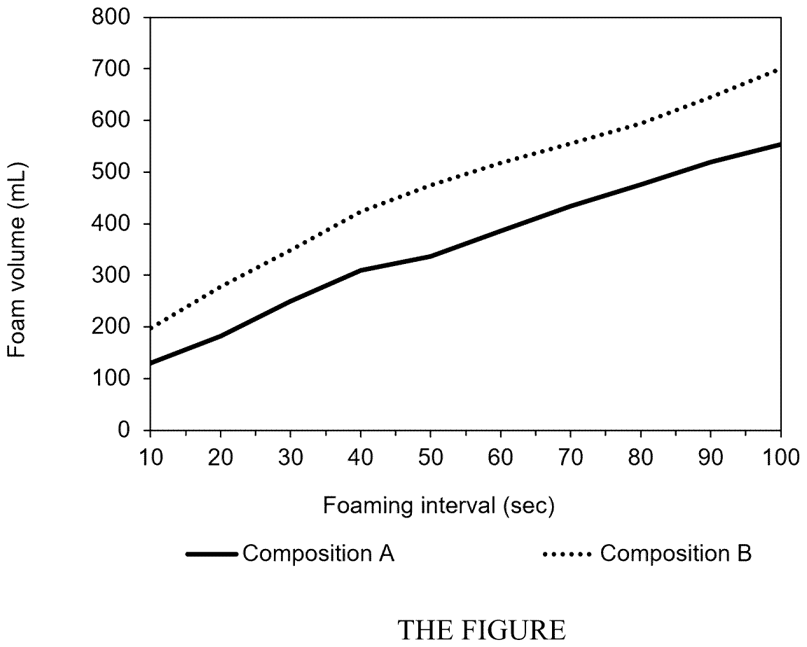

For evaluating the foaming performance, Compositions A and B were each diluted with water to a concentration of 0.5 wt % active surfactant matter. Each solution was adjusted to a total hardness of 100 dH (German hardness) and a pH value of 6. 300 mL of each test solution were tested for their foamability at 30° C. using a constant stirring speed of 1500 rpm for 10 sec. A total of 8 such measurement intervals was carried out for each test solution. All samples were tested in duplicate. FIG. 1 illustrates the foam volume over time for each test solution:

Measurement parameters: temperature: 30° C.±0,5° C.; sample volume/measurement: 300 mL; concentration of test sample: 0.5 wt % in water (10 °dH (German hardness)), pH adjusted with NaOH, stirring speed: 1500 rpm; stirring time: 10 sec; number of intervals: 8; number of repetitions: 2.

As seen in FIG. 1 the composition containing sophorolipid and lactic acid has a lower foam volume compared to composition B at a pH value of 6. This result is desired as a low foam volume is preferred in eye make-up remover and micellar water formulations.

### Shampoo Example Formulations:

Example formulations 1: Volume and Body Shampoo

| Ingredient | % |
|---|---|
| Aqua | ad 100 |
| Sodium Lauryl Sulfate | 7.0 |
| Sodium Laureth Sulfate | 5.0 |
| Cocamidopropyl Betaine | 2.0 |
| Composition A | 2.0 |
| Sodium Citrate | 1.5 |
| Sodium Xylenesulfonate | 1.5 |
| Sodium Chloride | 1.4 |
| Citric Acid | ad pH 5.5 |
| Sodium Levulinate, Sodium Benzoate | 1.0 |
| Hydroxypropyl Methylcellulose | 1.0 |
| Tetrasodium EDTA | 0.8 |
| Butylphenyl Methylpropional | 0.5 |
| Panthenol | 0.5 |
| Panthenyl Ethyl Ether | 0.3 |
| Starch Hydroxypropltrimonium chloride | 0.2 |
| Linalool | 0.1 |
| Hexyl Cinnamal | 0.1 |
| Limonene | 0.1 |
| Benzyl Salicylate | 0.2 |
| Magnesium Nitrate | 0.1 |
| Magnesium Chloride | 0.1 |
| Methylchloroisothiazolinone | 0.1 |
| Dyes | q.s. |

Example formulation 2: Repair Shampoo

| Ingredient | % |
|---|---|
| Aqua | ad 100 |
| Sodium Laureth Sulfate | 7.0 |
| Glycolipids | 3.0 |
| Composition A | 3.0 |
| Glycol Distearate | 2.5 |
| Cocamidopropyl Betaine | 2.0 |
| Sodium Citrate | 1.8 |
| Cocamide MEA | 1.8 |
| Sodium Xylenesulfonate | 1.5 |
| Dimethicone | 1.5 |
| Citric Acid | ad pH 5.0 |
| Sodium Benzoate | 0.7 |
| Polyquaternium-76 | 0.5 |
| Sodium Chloride | 0.9 |
| Glycerin | 0.5 |
| Tetrasodium EDTA | 0.3 |
| Butylphenyl Methylpropional | 0.2 |
| Hexyl Cinnamal | 0.1 |
| Linalool | 0.1 |
| alpha-Isomethyl Ionone | 0.1 |
| Geraniol | 0.2 |
| Hydrochloric Acid | 0.1 |
| Magnesium Nitrate | 0.1 |
| Methylchloroisothiazolinone | 0.1 |
| Magnesium Chloride | 0.1 |
| Methylisothiazolinone | 0.1 |
| Dyes | q.s. |

Example formulation 3: Anti-Dandruff Shampoo

| Ingredient | % |
|---|---|
| Aqua | ad 100 |
| Sodium Laureth Sulfate | 6.0 |
| Sodium Lauryl Sulfate | 5.0 |
| Composition A | 4.0 |
| Cocamide MEA | 4.0 |
| Zinc Carbonate | 3.0 |
| Glycol Distearate | 2.5 |
| Dimethicone | 1.0 |
| Zinc Pyrithione | 1.0 |
| Sodium Chloride | 0.6 |
| Cetyl Alcohol | 0.8 |
| Guar Hydroxypropyltrimonium Chloride | 0.3 |
| Sodium Xylenesulfonate | 0.5 |
| Magnesium Sulfate | 0.7 |
| Sodium Benzoate | 0.7 |
| Ammonium Laureth Sulfate | 0.3 |
| Citric Acid | ad pH 4.5 |
| Benzyl Alcohol | 0.2 |
| Methylchloroisothiazolinone | 0.1 |
| Methylisothiazolinone | 0.1 |
| Parfum, Dyes | q.s. |

Example formulation 4: Strengthening Shampoo

| Ingredient | % |
|---|---|
| Water | ad 100 |
| Sodium Lauryl Sulfate | 6.0 |
| Sodium Laureth Sulfate | 4.0 |
| Cocamidopropyl Betaine | 3.0 |
| Composition A | 3.0 |
| Glycol Distearate | 2.5 |
| Dimethicone | 2.0 |
| Sodium Citrate | 1.5 |
| Cocamide MEA | 1.0 |
| Sodium Xylenesulfonate | 1.0 |
| Citric Acid | ad pH 5.0 |
| Sodium Benzoate | 0.7 |
| Sodium Chloride | 0.8 |
| Tetrasodium EDTA | 0.3 |
| Polyquaternium-6 | 0.5 |
| Honey (Mel) | 0.5 |
| Prunus Armeniaca (Apricot) Fruit Extract | 0.5 |
| Methylchloroisothiazolinone | 0.1 |
| Methylisothiazolinone | 0.1 |
| Parfum, Dyes | q.s. |

Example formulation 5: Shampoo

| Ingredient | % |
|---|---|
| Aqua | ad 100 |
| Sodium Laureth Sulfate | 9.0 |
| Cocamidopropyl Betaine | 3.0 |
| Composition A | 3.0 |
| Glycerin | 2.5 |
| Sodium Chloride | 1.7 |
| Glycol Distearate | 1.5 |
| Panthenol | 0.2 |
| Propylene Glycol | 0.3 |
| Hydroxypropyl Guar Hydroxypropyltrimonium Chloride | 0.3 |
| Sodium Benzoate | 0.7 |
| PEG-55 Propylene Glycol Oleate | 0.5 |
| Citric Acid | ad pH 5.5 |
| Hydrolyzed Wheat Protein | 0.3 |
| Argania Spinosa Kernel Oil | 0.1 |
| Laureth-4 | 0.5 |
| Potassium Sorbate | 0.2 |
| Parfum, Dyes | q.s. |

Example formulation 6: Moisturising Shampoo

| Ingredient | % |
|---|---|
| Aqua | ad 100 |
| Sodium Laureth Sulfate | 8.0 |
| Cocamidopropyl Betaine | 3.0 |
| Sodium Chloride | 2.2 |
| Composition A | 2.0 |
| Hydrolyzed Keratin | 2.0 |
| Water Lily | 1.8 |
| Nymphaea Odorata Root Extract | 1.5 |
| Glyceryl Glucoside | 1.0 |
| Panthenol | 1.0 |
| Decyl Glucoside | 1.0 |
| Polyquaternium-10 | 0.2 |
| Guar Hydroxypropyltrimonium Chloride | 0.3 |
| PEG-40 Hydrogenated Castor Oil | 0.7 |
| Glycerin | 0.5 |
| Citric Acid | ad pH 5.0 |
| Sodium Benzoate | 1.2 |
| Propylene Glycol | 1.0 |
| Citronellol | 0.1 |
| Limonene | 0.1 |
| alpha-Isomethyl Ionone | 0.1 |
| Benzyl Alcohol | 0.2 |
| Dyes | q.s. |

Example formulation 7: Shampoo

| Ingredient | % |
|---|---|
| Aqua | ad 100 |
| Decyl Glucoside | 6.0 |
| Sodium Myreth Sulfate | 5.5 |
| Composition A | 3.0 |
| PEG-200 Hydrogenated Glyceryl Palmate | 2.5 |
| PEG-80 Sorbitan Laurate | 2.0 |
| PEG-90 Glycol Isostearate | 2.0 |
| Disodium PEG-5 Laurylcitrate Sulfosuccinate | 1.5 |
| Polyquaternium-10 | 1.2 |
| Hydrolyzed Silk | 1.2 |
| Laureth-2 | 1.2 |
| Citric Acid | ad pH 5.5 |
| Sodium Laureth Sulfate | 1.0 |
| PEG-40 Hydrogenated Castor Oil | 0.9 |
| Sodium Benzoate | 0.5 |
| Parfum, Dyes | q.s. |

Example formulation 8: Cleansing Oil Shampoo

| Ingredient | % |
|---|---|
| Aqua | ad 100 |
| Sodium Laureth Sulfate | 7.0 |
| MIPA-Laureth Sulfate | 4.0 |
| Sodium Chloride | 3.2 |
| Cocamidopropyl Betaine | 3.0 |
| Composition A | 3.0 |
| Glycerin | 2.5 |
| PEG-18 Castor Oil Dioleate | 2.0 |
| Propylene Glycol | 2.0 |
| PEG-55 Propylene Glycol Oleate | 1.7 |
| Behenamidopropyl Dimethylamine | 1.2 |
| Laureth-5 Carboxylic Acid | 1.0 |
| Persea Gratissima (Avocado) Oil | 1.0 |
| PPG-5-Ceteth-20 | 1.0 |
| Sodium Benzoate | 0.7 |
| Laureth-2 | 0.5 |
| Salicylic Acid | 0.3 |
| Linalool | 0.2 |
| alpha-Isomethyl Ionone | 0.1 |
| Limonene | 0.1 |
| Zea Mays (Corn) Germ Oil | 0.2 |
| Argania Spinosa Oil Kernel Oil | 0.1 |
| Camellia Oleifera Seed Oil | 0.1 |
| Sodium Hydroxide | 0.3 |
| Citric Acid | ad pH 5.0 |
| Parfum, Dyes | q.s. |

Example formulation 9: Shine Shampoo

| Ingredient | % |
|---|---|
| Aqua | ad 100 |
| Ammonium Lauryl Sulfate | 5.5 |
| Composition A | 2.0 |
| Cocamidopropyl Betaine | 2.0 |
| Glycolipids | 2.0 |
| Sodium Chloride | 2.0 |
| Cocamide MEA | 2.0 |
| Nacre Powder | 1.0 |
| Sodium Benzoate | 0.7 |
| Hydroxypropyl Guar Hydroxypropyltrimonium Chloride | 0.7 |
| Hydroxypropyltrimonium Hydrolyzed Wheat Protein | 0.7 |
| Sodium Hydroxide | 0.2 |
| Salicylic Acid | 0.5 |
| Limonene | 0.1 |
| Benzoic Acid | 0.5 |
| Linalool | 0.3 |
| Benzyl Alcohol | 0.5 |
| Tin Oxide | 0.2 |
| alpha-Isomethyl Ionone | 0.1 |
| Acrylates Copolymer | 0.1 |
| Citric Acid | ad pH 5.5 |
| Citronellol | 0.1 |
| Hexyl Cinnamal | 0.1 |
| Dyes | q.s. |

Example formulation 10: Shampoo

| Ingredient | % |
|---|---|
| Aqua | ad 100 |
| Sodium Cocoyl Isethionate | 3.5 |
| Sodium Lauryl Sulfoacetate | 3.5 |
| Disodium Laureth Sulfosuccinate | 3.5 |
| Cocamidopropyl Betaine | 3.0 |
| Composition A | 3.0 |
| Behenamidopropyl Dimethylamine | 0.9 |
| Sodium Lauroyl Sarcosinate | 1.0 |
| Glycol Distearate | 1.0 |
| C11-15 Pareth-7 | 1.0 |
| C12-13 Pareth-23 | 1.0 |
| C12-13 Pareth-3 | 1.0 |
| Decyl Glucoside | 1.0 |
| Tocopherol | 1.0 |
| Hydrogenated Coconut Acid | 1.0 |
| Sodium Chloride | 0.8 |
| Sodium Isethionate | 0.7 |
| Sodium Benzoate | 0.7 |
| Sodium Hydroxide | 0.3 |
| Phenoxyethanol | 0.6 |
| PPG-5 Ceteth-20 | 0.5 |
| PEG-55 Propylene Glycol Oleate | 0.5 |
| Trideceth-12 | 0.5 |
| Polyquaternium-7 | 0.5 |
| Polyquaternium-10 | 0.3 |
| Limonene | 0.1 |
| Benzoic Acid | 0.3 |
| Benzophenone-4 | 0.3 |
| Benzyl Salicylate | 0.2 |
| Linalool | 0.6 |
| Benzyl Alcohol | 0.6 |
| Amodimethicone | 0.5 |
| Propylene Glycol | 0.5 |
| Carbomer | 0.7 |
| Rosmarinus Officinalis (Rosemary) Leaf Oil | 0.7 |
| Methylparaben | 0.3 |
| Methylchloroisothiazolinone | 0.1 |
| Methylisothiazolinone | 0.1 |
| Citric Acid | ad pH 5.0 |
| Laureth-9 | 0.5 |
| Divinyldimethicone/Dimethicone Copolymer | 0.2 |
| Glycerin | 0.2 |
| Parfum, Dyes | q.s. |

Example formulation 11: Reinforcing Shampoo

| Ingredient | % |
|---|---|
| Aqua | ad 100 |
| Sodium Laureth Sulfate | 6.0 |
| Dimethicone | 3.0 |
| Sodium Chloride | 3.0 |
| Coco-Betaine | 3.0 |
| Composition A | 2.0 |
| Glycol Distearate | 1.0 |
| Guar Hydroxypropyltrimonium Chloride | 0.3 |
| Cocamide MIPA | 0.5 |
| Hydrolyzed keratin | 0.7 |
| Sodium Benzoate | 0.7 |
| Hydroxypropyltrimonium Hydrolyzed Wheat Protein | 0.3 |
| Sodium Cocoate | 0.7 |
| Sodium Hydroxide | 0.6 |
| Arginine | 0.7 |
| Salicylic Acid | 0.5 |
| Limonene | 0.2 |
| Linalool | 0.2 |
| 2-Oleamido-1.3-Octadecanediol | 0.1 |
| Carbomer | 0.5 |
| Methyl Cocoate | 0.5 |
| Citric Acid | ad pH 5.0 |
| Hexylene Glycol | 0.2 |
| Hexyl Cinnamal | 0.1 |
| Glyceryl Linoleate | 0.1 |
| Glyceryl Oleate | 0.2 |
| Glyceryl Linolenate | 0.2 |
| Dyes | q.s. |

Example formulation 12: Shampoo

| Ingredient | % |
|---|---|
| Aqua | ad 100 |
| Sodium Laureth Sulfate | 9.0 |
| Dimethicone | 3.5 |
| Sodium Chloride | 3.2 |
| Coco-Betaine | 3.0 |
| Glycol Distearate | 3.0 |
| Composition A | 2.0 |
| Guar Hydroxypropyltrimonium Chloride | 0.3 |
| Niacinamide | 0.1 |
| Cocamide MIPA | 2.0 |
| Saccharum Officinarum (Sugar Cane) Extract | 2.0 |
| Sodium Benzoate | 1.0 |
| Sodium Cocoate | 1.5 |
| Sodium Hydroxide | 0.6 |
| Salicylic Acid | 0.5 |
| Camellia Sinensis Extract/Camellia Sinensis Leaf Extract | 0.5 |
| Linalool | 0.2 |
| Benzyl Salicylate | 0.2 |
| Pyrus Malus (Apple) Fruit Extract | 0.3 |
| Carbomer | 0.5 |
| Pyridoxine HCl | 0.3 |
| Persea Gratissima (Avocado) Oil | 0.2 |
| Citric Acid | ad pH 5.5 |
| Butylphenyl Methylpropional | 0.1 |
| Methyl Cocoate | 0.3 |
| Hexylene Glycol | 0.1 |
| Hexyl Cinnamal | 0.1 |
| Dyes | q.s. |

Example formulation 13: Fortifying Shampoo

| Ingredient | % |
|---|---|
| Aqua | ad 100 |
| Ammonium Lauryl Sulfate | 6.0 |
| Cocamidopropyl Betaine | 3.0 |
| Sodium Chloride | 2.7 |
| Composition A | 2.0 |
| Niacinamide | 0.1 |
| Saccharum Officinarum (Sugar Cane) Extract | 0.9 |
| Cocos Nucifera (Coconut) Oil | 0.9 |
| Sodium Benzoate | 0.9 |
| Hydroxypropyl Guar Hydroxypropyltrimonium Chloride | 0.1 |
| Sodium Hydroxide | ad pH |
| Salicylic Acid | 0.4 |
| Limonene | 0.1 |
| Benzoic Acid | 0.4 |
| Linalool | 0.3 |
| Cinnamyl Alcohol | 0.2 |
| Pyrus Malus (Apple) Fruit Extract | 0.5 |
| Pyridoxine HCl | 0.2 |
| Bisabolol | 0.2 |
| Citric Acid | ad pH 5.0 |
| Citronellol | 0.1 |
| Hexylene Glycol | 0.2 |
| Dyes | q.s. |

Example formulation 14: Shampoo

| Ingredient | % |
|---|---|
| Aqua | ad 100 |
| Sodium Laureth Sulfate | 9.0 |
| Cocamidopropyl Betaine | 2.5 |
| Composition A | 2.5 |
| Sodium Chloride | 2.5 |
| Camellia Sinensis Leaf Extract | 2.0 |
| Guar Hydroxypropyltrimonium Chloride | 0.3 |
| Maltodextrin | 1.2 |
| Disodium EDTA | 1.0 |
| PPG-12 | 0.7 |
| Citric Acid | ad pH 5.0 |
| Sodium Hydroxide | 0.3 |
| Sodium Benzoate | 0.7 |
| Butylphenyl Methylpropional | 0.2 |
| Geraniol | 0.2 |
| Hexyl Cinnamal | 0.1 |
| Limonene | 0.1 |
| Linalool | 0.1 |
| Dyes | q.s. |

Example formulation 15: Brunette Shampoo

| Ingredient | % |
|---|---|
| Aqua | ad 100 |
| Sodium Laureth Sulfate | 8.5 |
| Cocamidopropyl Betaine | 4.0 |
| Sodium Chloride | 3.5 |
| Composition A | 2.0 |
| Selaginella Lepidophylla Aerial Extract | 2.0 |
| Gluconolactone | 1.8 |
| Glycerin | 1.8 |
| Dimethiconol | 1.0 |
| Guar Hydroxypropyltrimonium Chloride | 0.5 |
| TEA-Dodecylbenzenesulfonate | 1.0 |
| Maltodextrin | 1.0 |
| Disodium EDTA | 0.7 |
| Carbomer | 0.9 |
| PPG-12 | 0.7 |
| Citric Acid | ad pH 5.5 |
| Sodium Hydroxide | 0.9 |
| TEA-Sulfate | 0.7 |
| Triethanolamine | 0.5 |
| DMDM Hydantoin | 0.1 |
| Sodium Benzoate | 0.7 |
| Methylchloroisothiazolinone | 0.1 |
| Methylisothiazolinone | 0.1 |
| alpha-Isomethyl Ionone | 0.1 |
| Butylphenyl Methylpropional | 0.2 |
| Hexyl Cinnamal | 0.1 |
| Limonene | 0.1 |
| Linalool | 0.1 |
| Dyes | q.s. |

Example formulation 16: 2 in 1 Shampoo

| Ingredient | % |
|---|---|
| Aqua | ad 100 |
| Sodium Laureth Sulfate | 8.0 |
| PEG-200 Hydrogenated Glyceryl Palmate | 3.0 |
| Composition A | 3.0 |
| Disodium Cocoamphodiacetate | 3.0 |
| Polysorbate 20 | 2.5 |
| Glycerin | 2.5 |
| Sodium Chloride | 2.0 |
| PEG-7 Glyceryl Cocoate | 2.0 |
| Sodium Benzoate | 1.0 |
| Sodium Laureth-8 Sulfate | 1.0 |
| Sodium Oleth Sulfate | 1.0 |
| Sodium Hydroxide | 0.3 |
| PPG-5 Ceteth-20 | 1.0 |
| PEG-55 Propylene Glycol Oleate | 0.5 |
| Polyquaternium-10 | 0.2 |
| Salicylic Acid | 0.3 |
| Maltodextrin | 0.2 |
| Propylene Glycol | 0.2 |
| Disodium Ricinoleamido MEA-Sulfosuccinate | 0.1 |
| Citric Acid | ad pH 5.5 |
| Prunus Armeniaca (Apricot) Kernel Oil | 0.1 |
| Parfum, Dyes | q.s. |

Example formulation 17: Shampoo

| Ingredient | % |
|---|---|
| Aqua | ad 100 |
| Sodium Laureth Sulfate | 9.0 |
| Cocamidopropyl Betaine | 3.0 |
| Sodium Chloride | 2.5 |
| Glycol Distearate | 2.5 |
| Hydrolyzed Keratin | 2.0 |
| Simmondsia Chinensis (Jojoba) Seed Oil | 2.0 |
| Composition A | 2.0 |
| Argania Spinosa Kernel Oil | 0.5 |
| Persea Gratissima (Avocado) Oil | 0.5 |
| Rosmarinus Officinalis (Rosemary) Leaf Extract | 0.3 |
| Helianthus Annuus (Sunflower) Seed Oil | 0.3 |
| Cocamide MEA | 0.4 |
| Guar Hydroxypropyltrimonium Chloride | 0.2 |
| Polyquaternium-47 | 0.2 |
| PEG-20 Castor Oil | 0.5 |
| Phenethyl Benzoate | 0.7 |
| Disodium EDTA | 0.2 |
| Methylchloroisothiazolinone | 0.3 |
| Methylisothiazolinone | 0.3 |
| Benzyl Salicylate | 0.1 |
| Hexyl Cinnamal | 0.1 |
| Citronellol | 0.1 |
| Dyes | q.s. |

Example formulation 18: Foam Shampoo

| Ingredient | % |
|---|---|
| Aqua | ad 100 |
| Sodium Laureth Sulfate | 5.0 |
| Isobutane | 5.0 |
| Decyl Glucoside | 5.0 |
| Propane | 3.5 |
| Sodium Cocoamphoacetate | 3.5 |
| Composition A | 3.5 |
| Glycolipids | 1.0 |
| Panthenol | 0.2 |
| Polyquaternium-10 | 0.2 |
| Hydroxypropyl Guar Hydroxypropyltrimonium Chloride | 0.1 |
| Sodium PCA | 0.5 |
| Octyldodecyl PCA | 0.5 |
| Maltodextrin | 0.5 |
| Tilia Cordata Flower Extract | 0.7 |
| Sodium Chloride | 0.9 |
| Citric Acid | ad pH 5.0 |
| Glycerin | 0.9 |
| Propylene Glycol | 0.3 |
| Butane | 0.7 |
| Sodium Acetate | 0.7 |
| Sorbitol | 0.5 |
| Isopropyl Alcohol | 0.5 |
| Dyes | q.s. |

Example formulation 19: Anti-Dandruff Shampoo

| Ingredient | % |
|---|---|
| Aqua | ad 100 |
| Sodium Laureth Sulfate | 10.0 |
| Cocamidopropyl Betaine | 3.0 |
| Acrylates Copolymer | 2.5 |
| Sodium Lauroyl Glutamate | 2.5 |
| Zinc Pyrithione | 2.0 |
| Composition A | 2.0 |
| Stearamidopropyl Dimethylamine | 1.0 |
| Piroctone Olamine | 0.7 |
| Hydroxypropyl Guar Hydroxypropyltrimonium Chloride | 0.2 |
| Propylene Glycol | 0.5 |
| Sodium PCA | 0.2 |
| Citrus Medica Limonum (Lemon) Peel Extract | 0.5 |
| Citric Acid | ad pH 5.0 |
| Glycol Distearate | 0.8 |
| PPG-9 | 0.2 |
| Laureth-4 | 0.2 |
| Sodium Chloride | 0.9 |
| Sodium Hydroxide | 0.3 |
| Sodium Naphthalenesulfonate | 0.7 |
| Sorbitol | 0.7 |
| DMDM Hydantoin | 0.1 |
| Sodium Benzoate | 0.8 |
| Limonene | 0.1 |
| Linalool | 0.1 |
| Butylphenyl Methylpropional | 0.1 |
| alpha-Isomethyl Ionone | 0.1 |
| Parfum, Dyes | q.s. |

Example formulation 20: Shampoo

| Ingredient | % |
|---|---|
| Aqua | ad 100 |
| Sodium Laureth Sulfate | 5.0 |
| Disodium Cocoamphodiacetate | 2.0 |
| Composition A | 2.0 |
| Laureth-2 | 0.3 |
| Panthenol | 0.2 |
| Niacinamide | 0.1 |
| Sodium Chloride | 0.7 |
| Citric Acid | ad pH 5.0 |
| PEG-7 Glyceryl Cocoate | 0.5 |
| Sodium Benzoate | 0.5 |
| Polyquaternium-10 | 0.7 |
| Salicylic Acid | 0.5 |
| Hexyl Salicylate | 0.2 |
| Benzyl Salicylate | 0.1 |
| Hexyl Cinnamal | 0.1 |
| Linalool | 0.1 |
| Limonene | 0.1 |
| Propylene Glycol | 0.2 |
| Dyes | q.s. |

Example formulation 21: Intensive Care Oil-Shampoo

| Ingredient | % |
|---|---|
| Aqua | ad 100 |
| Sodium Laureth Sulfate | 8.0 |
| Disodium Cocoamphodiacetate | 3.0 |
| Composition A | 0.5 |
| Prunus Armeniaca (Apricot) Kernel Oil | 2.5 |
| Hydrolyzed Keratin | 2.5 |
| Panthenol | 2.4 |
| Sodium Chloride | 2.2 |
| Citric Acid | ad pH 5.5 |
| Stearamidopropyl Dimethylamine | 1.2 |
| Polyquaternium-10 | 0.5 |
| Sodium Benzoate | 0.5 |
| PEG-7 Glyceryl Cocoate | 0.7 |
| Laureth-2 | 0.4 |
| PEG-40 Hydrogenated Castor Oil | 0.4 |
| Propylene Glycol | 0.8 |
| PEG-55 Propylene Glycol Oleate | 0.7 |
| Hexyl Cinnamal | 0.1 |
| Butylphenyl Methylpropional | 0.2 |
| Benzyl Salicylate | 0.1 |
| Linalool | 0.2 |
| Benzyl Alcohol | 0.5 |
| Limonene | 0.1 |
| Sodium Acetate | 0.2 |
| Dyes | q.s. |

Example formulation 22: Shampoo

| Ingredient | % |
|---|---|
| Aqua | ad 100 |
| Sodium Laureth Sulfate | 8.5 |
| Disodium Cocoamphodiacetate | 3.5 |
| Sodium Chloride | 3.5 |
| Composition A | 3.0 |
| Hydrolyzed Collagen | 1.0 |
| Panthenol | 0.4 |
| Cocodimonium Hydroxypropyl Hydrolyzed Keratin | 0.3 |
| Hydrolyzed Keratin | 0.3 |
| Citric Acid | ad pH 5.5 |
| Sodium Benzoate | 0.7 |
| Polyquaternium-10 | 0.2 |
| Hexyl Cinnamal | 0.1 |
| Limonene | 0.1 |
| Linalool | 0.1 |
| Benzyl Salicylate | 0.1 |
| Benzyl Alcohol | 0.2 |
| Propylene Glycol | 0.2 |
| Dyes | q.s. |

Example formulation 23: Shampoo

| Ingredient | % |
|---|---|
| Aqua | ad 100 |
| Sodium Laureth Sulfate | 8.0 |
| Cocamidopropyl Betaine | 3.0 |
| Composition A | 3.0 |
| PEG-7 Glyceryl Cocoate | 2.5 |
| Dimethicone | 2.5 |
| Prunus Armeniaca (Apricot) Kernel Oil | 2.5 |
| Panthenol | 0.3 |
| Cocodimonium Hydroxypropyl Hydrolyzed Keratin | 0.7 |
| Hydrolyzed Keratin | 0.7 |
| Sodium Chloride | 0.8 |
| Cocamide MEA | 0.5 |
| Glycol Distearate | 0.2 |
| Sodium Benzoate | 0.7 |
| Citric Acid | ad pH 5.5 |
| Polyquaternium-10 | 0.5 |
| Laureth-4 | 0.4 |
| PEG-40 Hydrogenated Castor Oil | 0.2 |
| Hydrogenated Castor Oil | 0.7 |
| Laureth-7 | 0.5 |
| Hexyl Cinnamal | 0.1 |
| Propylene Glycol | 0.8 |
| Hexyl Salicylate | 0.3 |
| Glycerin | 0.5 |
| Linalool | 0.2 |
| Limonene | 0.1 |
| Dyes | q.s. |

Example formulation 24: Shampoo

| Ingredient | % |
|---|---|
| Aqua | ad 100 |
| Sodium Coco Sulfate | 6.0 |
| Lauryl Glucoside | 4.0 |
| Glycerin | 3.5 |
| Sodium Lactate | 2.5 |
| Composition A | 2.0 |
| Stearamidopropyl Dimethylamine | 1.0 |
| Betaine | 0.8 |
| Sodium Chloride | 0.9 |
| Simmondsia Chinensis Seed Oil | 0.8 |
| Sodium Cocoyl Glutamate | 0.7 |
| Disodium Cocoyl Glutamate | 0.5 |
| Xanthan Gum | 0.5 |
| Hydrated Silica | 0.5 |
| Parfum, Dyes | q.s. |

Example formulation 25: Shampoo for Children

| Ingredient | % |
|---|---|
| Aqua | ad 100 |
| Sodium Coco Sulfate | 7.0 |
| Decyl Glucoside | 5.0 |
| Lactis Proteinum | 3.5 |
| Sorbitan Caprylate | 3.0 |
| Composition A | 3.0 |
| Glycerin | 2.5 |
| Sodium Lactate | 2.5 |
| Alcohol | 2.0 |
| Hydrolyzed Wheat Protein | 0.7 |
| Hydrolyzed Wheat Starch | 0.7 |
| Sodium Chloride | 0.9 |
| Limonene | 0.1 |
| Citral | 0.1 |
| Phenethyl Alcohol | 0.1 |
| Dyes | q.s. |

Example formulation 26: Intensive Cream Shampoo

| Ingredient | % |
|---|---|
| Aqua | ad 100 |
| Sodium Laureth Sulfate | 8.5 |
| Cocamidopropyl Betaine | 3.0 |
| Composition A | 2.5 |
| Sodium Chloride | 2.5 |
| Panthenol | 0.5 |
| Hydrolyzed Keratin | 0.8 |
| Argania Spinosa Kernel Oil | 0.5 |
| Cocamide MEA | 0.5 |
| Glycol Distearate | 0.5 |
| PEG-7 Glyceryl Cocoate | 0.4 |
| Sodium Benzoate | 0.7 |
| Citric Acid | ad pH 5.0 |
| Laureth-4 | 0.3 |
| PEG-40 Hydrogenated Castor Oil | 0.4 |
| Hydrogenated Castor Oil | 0.4 |
| Styrene/Acrylates Copolymer | 0.5 |
| Dimethicone | 0.8 |
| Polyquaternium-7 | 0.9 |
| PEG-12 Dimethicone | 0.5 |
| Guar Hydroxypropyltrimonium Chloride | 0.3 |
| Benzyl Salicylate | 0.1 |
| Propylene Glycol | 0.8 |
| Glycerin | 0.5 |
| Limonene | 0.1 |
| Linalool | 0.1 |
| PEG-14m | 0.1 |
| Dyes | q.s. |

Example formulation 27: Shampoo

| Ingredient | % |
|---|---|
| Aqua | ad 100 |
| Sodium Laureth Sulfate | 7.5 |
| Cocamidopropyl Betaine | 3.0 |
| Composition A | 2.5 |
| Sodium Chloride | 2.0 |
| Sodium Xylenesulfonate | 1.7 |
| Cocamide MEA | 1.5 |
| Sodium Citrate | 1.5 |
| Citric Acid | ad pH 5.0 |
| Dimethiconol | 1.0 |
| Cassia Hydroxypropyltrimonium Chloride | 0.3 |
| Sodium Benzoate | 0.9 |
| Disodium EDTA | 0.7 |
| Panthenol | 0.7 |
| Panthenyl Ethyl Ether | 0.3 |
| Persea Gratissima (Avocado) Oil | 0.2 |
| Methylchloroisothiazolinone | 0.1 |
| Methylisothiazolinone | 0.1 |
| Parfum, Dyes | q.s. |

Example formulation 28: Caffeine Shampoo

| Ingredient | % |
|---|---|
| Aqua | ad 100 |
| Sodium Laureth Sulfate | 6.0 |
| Laureth-2 | 4.0 |
| Disodium Laureth Sulfosuccinate | 2.5 |
| Sodium Lauroyl Glutamate | 2.5 |
| Composition A | 2.0 |
| Sodium Chloride | 2.0 |
| Caffeine | 1.5 |
| Panthenol | 0.2 |
| PEG-120 Methyl Glucose Dioleate | 0.5 |
| Hydrolysed Wheat Protein | 0.7 |
| Citric Acid | ad pH 5.5 |
| Sodium Citrate | 0.5 |
| Menthol | 0.7 |
| PEG-40 Hydrogenated Castor Oil | 0.4 |
| Potassium Sorbate | 0.3 |
| Polyquaternium-7 | 0.4 |
| Disodium EDTA | 0.5 |
| Sodium Benzoate | 0.7 |
| Zinc PCA | 0.3 |
| Niacinamide | 0.1 |
| Limonene | 0.1 |
| Tocopherol | 0.1 |
| Phenoxyethanol | 0.5 |
| Methylparaben | 0.3 |
| Propylparaben | 0.3 |
| Dyes | q.s. |

Example formulation 29: Power Grey Shampoo

| Ingredient | % |
|---|---|
| Aqua | ad 100 |
| Sodium Laureth Sulfate | 6.5 |
| Sodium Cocoamphoacetate | 4.5 |
| PEG-3 Distearate | 3.0 |
| Cocamidopropyl Betaine | 3.0 |
| Composition A | 2.5 |
| Sodium Chloride | 2.5 |
| Caffeine | 1.5 |
| Panthenol | 0.2 |
| Phenoxyethanol | 0.7 |
| Coco Glucoside | 0.9 |
| Glyceryl Oleate | 0.4 |
| Phosphoric Acid | 0.2 |
| PEG-120 Methyl Glucose Dioleate | 0.9 |
| Menthol | 0.9 |
| Polyquaternium-7 | 0.7 |
| Sodium Hydroxide | 0.3 |
| Citric Acid | ad pH 5.5 |
| Niacinamide | 0.1 |
| Zinc PCA | 0.5 |
| Limonene | 0.1 |
| Sodium Benzoate | 0.2 |
| Methylparaben | 0.3 |
| Propylparaben | 0.3 |
| Dyes | q.s. |

Example formulation 30: Shampoo

| Ingredient | % |
|---|---|
| Aqua | ad 100 |
| Lauryl Glucoside | 6.5 |
| Cocamidopropyl Betaine | 3.0 |
| Coco Glucoside | 2.5 |
| Composition A | 2.0 |
| Glyceryl Oleate | 1.5 |
| Sodium Cocoyl Glutamate | 1.0 |
| Panthenol | 0.5 |
| Polyquaternium-10 | 0.3 |
| Chamomilla Recutita Extract | 0.5 |
| Glyceryl Caprylate | 0.3 |
| p-Anisic Acid | 0.05 |
| Citric Acid | ad pH 5.0 |
| Parfum, Dyes | q.s. |

Example formulation 31: Caffeine Shampoo

| Ingredient | % |
|---|---|
| Aqua | ad 100 |
| Sodium Coco Sulfate | 6.0 |
| Glycerin | 5.5 |
| Lauryl Glucoside | 4.5 |
| Cocamidopropyl Betaine | 3.0 |
| Composition A | 2.5 |
| Lauroyl Sarcosine | 2.0 |
| Caffeine | 1.0 |
| Xanthan Gum | 1.0 |
| Hydroxypropyl Guar Hydroxypropyltrimonium Chloride | 0.3 |
| Alcohol | 0.8 |
| Coffea Arabica Bean Extract | 0.3 |
| Panthenyl Ethyl Ether | 0.4 |
| Panthenol | 0.2 |
| Limonene | 0.1 |
| Linalool | 0.1 |
| Dyes | q.s. |

Example formulation 32: Shampoo

| Ingredient | % |
|---|---|
| Aqua | ad 100 |
| Sodium Laureth Sulfate | 6.5 |
| Cocamidopropyl Betaine | 3.0 |
| Sodium Chloride | 3.0 |
| Composition A | 2.0 |
| Panthenol | 0.2 |
| Benzophenone-4 | 0.9 |
| Glycol Distearate | 0.7 |
| Laureth-4 | 0.8 |
| Glycerin | 0.9 |
| Formic Acid | 0.2 |
| Decyl Glucoside | 0.9 |
| Polyquaternium-10 | 0.4 |
| Niacinamide | 0.1 |
| Sodium Benzoate | 0.1 |
| Potassium Sorbate | 0.2 |
| Citric Acid | ad pH 5.0 |
| Parfum, Dyes | q.s. |

Example formulation 33: Shampoo

| Ingredient | % |
|---|---|
| Aqua | ad 100 |
| Sodium Laureth Sulfate | 8.5 |
| Cocamidopropyl Betaine | 3.0 |
| Sodium Chloride | 2.0 |
| Disodium Cocoamphodiacetate | 2.0 |
| Composition A | 2.0 |
| Sodium Benzoate | 0.7 |
| Panthenol | 0.2 |
| Sodium Laureth-8 Sulfate | 0.7 |
| Citric Acid | ad pH 5.0 |
| Sodium Cocoyl Glutamate | 0.8 |
| Glycol Distearate | 0.9 |
| Styrene/Acrylates Copolymer | 0.5 |
| Sodium Oleth Sulfate | 0.7 |
| Coco Glucoside | 0.9 |
| Cocamide MEA | 0.7 |
| Isopropyl Alcohol | 0.2 |
| Parfum, Dyes | q.s. |

Example formulation 34: Volume Shampoo

| Ingredient | % |
|---|---|
| Aqua | ad 100 |
| Sodium Laureth Sulfate | 7.0 |
| Cocamidopropyl Betaine | 3.0 |
| Coco Glucoside | 3.0 |
| Composition A | 2.0 |
| Nymphaea Coerulea Flower Extract | 2.0 |
| Hydrolyzed Rice Protein | 2.0 |
| Cocamide MEA | 2.0 |
| Polyquaternium-10 | 0.5 |
| Malic Acid | ad pH 5.5 |
| Disodium EDTA | 0.5 |
| Propylene Glycol | 0.5 |
| Benzophenone-4 | 0.3 |
| Ethylhexyl Methoxycinnamate | 0.3 |
| Sodium Chloride | 0.9 |
| Benzyl Alcohol | 0.9 |
| PPG-9 | 0.2 |
| Methylisothiazolinone | 0.1 |
| Methylparaben | 0.1 |
| Propylparaben | 0.1 |
| Parfum, Dyes | q.s. |

Example formulation 35: Shampoo

| Ingredient | % |
|---|---|
| Aqua | ad 100 |
| Sodium Laureth Sulfate | 6.5 |
| Coco Glucoside | 5.0 |
| Cocamidopropyl Betaine | 3.0 |
| Composition A | 3.0 |
| Glycol Distearate | 2.8 |
| Dimethicone | 1.7 |
| Laureth-23 | 1.5 |
| Laureth-4 | 1.5 |
| Polyquaternium-7 | 0.5 |
| Cocamide MEA | 0.7 |
| Cetyl Alcohol | 0.4 |
| Simmondsia Chinensis Seed Oil | 0.8 |
| Panthenol | 0.3 |
| Malic Acid | ad pH 5.5 |
| Glycine | 0.7 |
| Benzyl Alcohol | 0.7 |
| Disodium EDTA | 0.5 |
| Sodium Hydroxide | 0.2 |
| Guar Hydroxypropyltrimonium Chloride | 0.5 |
| Ethylhexyl Methoxycinnamate | 0.7 |
| Methylparaben | 0.3 |
| Propylparaben | 0.3 |
| PPG-9 | 0.2 |
| Benzophenone-4 | 0.3 |
| Sodium Chloride | 0.8 |
| DMDM Hydantoin | 0.1 |
| Hexyl Cinnamal | 0.1 |
| Limonene | 0.1 |
| Dyes | q.s. |

Example formulation 36: Shampoo

| Ingredient | % |
|---|---|
| Aqua | ad 100 |
| Sodium Laureth Sulfate | 5.0 |
| Sodium Lauryl Sulfate | 5.0 |
| Cocamidopropyl Betaine | 3.0 |
| Composition A | 2.5 |
| Olea Europaea Fruit Oil | 2.0 |
| Cocamide MEA | 2.0 |
| Sodium Chloride | 1.9 |
| Glycol Distearate | 1.7 |
| Glycolipids | 1.0 |
| Benzyl Alcohol | 1.0 |
| Polyquaternium-10 | 0.5 |
| Hydroxypropyl Methylcellulose | 0.5 |
| Silicone Quaternium-18 | 0.9 |
| Malic Acid | ad pH 5.5 |
| Disodium EDTA | 0.5 |
| Sodium Xylenesulfonate | 0.7 |
| Trideceth-6 | 0.7 |
| Trideceth-12 | 0.5 |
| DMDM Hydantoin | 0.2 |
| Methylparaben | 0.3 |
| Propylparaben | 0.3 |
| Linalool | 0.1 |
| Hexyl Cinnamal | 0.1 |
| Butylphenyl Methylpropional | 0.1 |
| Limonene | 0.1 |
| Dyes | q.s. |

Example formulation 37: Shampoo

| Ingredient | % |
|---|---|
| Aqua | ad 100 |
| Sodium Laureth Sulfate | 3.0 |
| Sodium Lauryl Sulfate | 3.5 |
| Glycol Distearate | 3.3 |
| Dimethicone | 3.0 |
| Cocamidopropyl Betaine | 2.5 |
| Composition A | 2.0 |
| Benzyl Alcohol | 0.7 |
| Betaine | 0.4 |
| Cetyl Alcohol | 0.7 |
| Cocamide MEA | 0.7 |
| Glyceryl Oleate | 0.5 |
| Propylene Glycol | 0.6 |
| Menthol | 0.7 |
| Guar Hydroxypropyltrimonium Chloride | 0.4 |
| Butylene Glycol | 0.5 |
| Menthyl Lactate | 0.2 |
| Disodium EDTA | 0.5 |
| Tocopheryl Acetate | 0.1 |
| Laureth-4 | 0.7 |
| Niacinamide | 0.1 |
| Maltodextrin | 0.2 |
| Sodium Ascorbyl Phosphate | 0.1 |
| PPG-9 | 0.2 |
| Salicylic Acid | 0.7 |
| Pyridoxine HCl | 0.1 |
| Malic Acid | ad pH 5.0 |
| Laureth-23 | 0.5 |
| Silica | 0.7 |
| Sodium Chloride | 0.7 |
| Sodium Hydroxide | 0.3 |
| Methylchloroisothiazolinone | 0.1 |
| Methylisothiazolinone | 0.1 |
| Parfum, Dyes | q.s. |

Example formulation 38: Shampoo

| Ingredient | % |
|---|---|
| Aqua | ad 100 |
| Cocamidopropyl Betaine | 7.0 |
| Sodium Methyl Cocoyl Taurate | 4.0 |
| Glycolipids | 2.0 |
| Composition A | 1.0 |
| Sodium Chloride | 2.0 |
| Sodium C14-16 Olefin Sulfonate | 1.0 |
| PEG-120 Methyl Glucose Dioleate | 1.0 |
| Glycol Distearate | 1.0 |
| Sodium Benzoate | 1.0 |
| Caprylyl/Capryl Glucoside | 0.7 |
| PEG-7 Glyceryl Cocoate | 0.7 |
| Citric Acid | ad pH 5.3 |
| Parfum | 0.2 |
| Polyquaternium-10 | 0.2 |
| Coconut Acid | 0.1 |
| Laureth-4 | 0.1 |
| Hydrogenated Castor Oil | 0.1 |
| Glycerin | 0.1 |
| Lactic Acid | 0.1 |
| Propylene Glycol | 0.1 |
| Hydrolyzed Keratin | 0.1 |
| Ceramide NP | 0.05 |
| Panthenol | 0.05 |

Example formulation 39: Conditioning Shampoo

| Ingredient | % |
|---|---|
| Aqua | ad 100 |
| Sodium Cocoamphoacetate | 5.0 |
| Cocamidopropyl Betaine | 4.0 |
| Glycolipids | 1.5 |
| Composition A | 1.5 |
| Starch Hydroxypropltrimonium chloride | 1.2 |
| Isostearamide MIPA; Glyceryl Laurate | 1.2 |
| Isoamyl Laurate | 1.0 |
| Bis-(Isostearoyl/Oleoyl Isopropyl) Dimonium Methosulfate | 0.5 |
| Citric Acid | 3.0 |
| Perfume, Preservative, Dyes | q.s. |

Example formulation 40: Conditioning Shampoo

| Ingredient | % |
|---|---|
| Aqua | ad 100 |
| Sodium Laureth Sulfate | 7.0 |
| Cocamidopropyl Betaine | 1.5 |
| Composition As | 1.5 |
| Palmitamidopropyltrimonium Chloride | 1.0 |
| Polysilicone-19 | 1.0 |
| PEG-18 Glyceryl Oleate/Cocoate | 2.5 |
| Citric Acid | ad pH 5.5 |
| Perfume, Preservative | q.s. |

Example formulation 41: Conditioning Shampoo

| Ingredient | % |
|---|---|
| Aqua | ad 100 |
| Sodium Cocoamphoacetate | 5.0 |
| Cocamidopropyl Betaine | 3.0 |
| Disodium Lauryl Sulfosuccinate | 3.0 |
| Glycolipids | 1.5 |
| Composition As | 1.5 |
| Palmitamidopropyltrimonium Chloride | 2.0 |
| Isostearamide MIPA, Glyceryl Laurate | 1.0 |
| PEG-200 Hydrogenated Glyceryl Palmate; | 1.0 |
| PEG-7 Glyceryl Cocoate | |
| Methoxy PEG/PPG-7/3 Aminopropyl Dimethicone | 0.5 |
| Starch Hydroxypropltrimonium chloride | 0.3 |
| Polyquaternium-10 | 0.2 |
| Citric Acid | ad pH 5.5 |
| Perfume, Preservative | q.s. |

Example formulation 42: Conditioning Shampoo

| Ingredient | % |
|---|---|
| Aqua | ad 100 |
| Sodium Laureth Sulfate | 6.0 |
| Cocamidopropyl Betaine | 3.0 |
| Composition A | 3.0 |
| Glycol Distearate, Laureth-4, Cocamidopropyl Betaine | 2.0 |
| PEG-120 Methyl Glucose Dioleate | 1.7 |
| Composition A | 1.5 |
| Sorbitan Sesquicaprylate | 1.0 |
| Isoamyl Laurate | 1.0 |
| Sodium Chloride | 1.0 |
| Silicone-Quaternium-22 | 0.8 |
| Glycolipids | 0.5 |
| Hydroxypropyl Guar Hydroxypropyltrimonium Chloride | 0.5 |
| Creatine | 0.5 |
| Citric Acid | ad pH 5.5 |
| Perfume, Preservative | q. s. |

Example formulation 43: Natural Shampoo

| Ingredient | % |
|---|---|
| Aqua | ad 100 |
| Sodium Coco-Sulfate | 9.0 |
| Glycerin | 5.0 |
| Lauryl Glucoside | 2.5 |
| Composition A | 2.5 |
| Sodium Lactate | 1.0 |
| Betaine | 1.0 |
| Hamamelis Virginiana Leaf Extract | 1.0 |
| Alcohol Denat. | 1.0 |
| Sodium/Disodium Cocoyl Glutamate | 0.8 |
| Maris Sal | 0.5 |
| Dipotassium Glycyrrhizate | 0.5 |
| Glycerophosphocholine | 0.1 |
| Parfum, Preservative | q. s. |

Example formulation 44: Natural Shampoo

| Ingredient | % |
|---|---|
| Aqua | ad 100 |
| Sodium Coco-sulfate | 6.0 |
| Glycerin | 5.0 |
| Coco-glucoside | 4.0 |
| Lauryl Glucoside | 3.0 |
| Composition A | 2.0 |
| Glyceryl Oleate | 1.5 |
| Sodium Chloride | 1.0 |
| Calendula Officinalis Flower Extract | 0.5 |
| Parfum | 0.5 |
| Starch Hydroxypropyltrimonium chloride | 0.3 |
| Glyceryl Undecylenate | 0.3sarch |
| Polyglyceryl-4 Caprate | 0.3 |
| Xanthan Gum | 0.3 |
| Locust bean Gum | |
| Lysolecithin | 0.2 |
| Coco-Caprylate | 0.1 |
| Magnolia Officinalis Bark Extract | 0.1 |
| Tocopherol | 0.1 |
| Squalene | 0.1 |

Example formulation 45: Natural Shampoo

| Ingredient | % |
|---|---|
| Aqua | ad 100 |
| Sodium Lauroyl Methyl Isethionate | 5.0 |
| Disodium Cocoamphodipropionate | 4.0 |
| Acrylates Copolymer | 2.0 |
| Hydroxyethylcellulose | 2.0 |
| Composition A | 2.0 |
| Cocos Nucifera Oil | 0.8 |
| Panthenol | 0.4 |
| Tocopherol | 0.4 |
| Tocopheryl Acetate | 0.2 |
| Cetyl Triethylmonium Dimethicone PEG-8 Succinate | 0.2 |
| Glycol Distearate | 0.2 |
| Laureth-4 | 0.2 |
| Cocamidopropyl Betaine | 0.2 |
| Ethylhexyl Salicylate | 0.1 |
| Disodium EDTA | 0.1 |
| Ethylhexylglycerin | 0.1 |
| Glycerin | 0.1 |
| Hexylene Glycol | 0.1 |
| Sodium Coco PG-dimonium Chloride Phosphate | 0.1 |
| BHT | 0.1 |
| Citric Acid | 0.1 |
| Phenoxyethanol | 0.1 |
| Formic Acid | 0.1 |
| Parfum | q.s. |

Example formulation 46: Natural Shampoo

| Ingredient | % |
|---|---|
| Aqua | ad 100 |
| Moroccan Lava Clay | 5.0 |
| Behenyl Alcohol | 3.0 |
| Glycerin | 2.5 |
| Glyceryl Stearate Citrate | 2.0 |
| Lauryl Glucoside | 2.0 |
| Coco-glucoside | 2.0 |
| Helianthus Annuus Hybrid Oil | 1.5 |
| Prunus Armeniaca Kernel Oil | 1.5 |
| Composition A | 1.0 |
| Glycolipids | 1.0 |
| Argania Spinosa Kernel Oil | 1.0 |
| Magnolia Officinalis Bark Extract | 1.0 |
| Triethyl Citrate, Glyceryl Caprylate Benzoic Acid | 1.0 |
| Xanthan Gum | 0.8 |
| Sodium Lactate | 0.5 |
| Benzyl Alcohol | 0.3 |
| Lactic Acid | 0.2 |
| Parfum | q.s. |

Example formulation 47: Natural Shampoo

| Ingredient | % |
|---|---|
| Aqua | ad 100 |
| Cocamidopropyl Betaine | 7.0 |
| Cetrimonium Chloride | 5.0 |
| Decyl Glucoside | 5.0 |
| Sodium Chloride | 2.0 |
| Composition A | 1.0 |
| Sorbitan Caprylate | 0.8 |
| Cocos Nucifera Oil | 0.8 |
| Inulin | 0.5 |
| Benzyl Alcohol | 0.2 |
| Cetearyl Alcohol | 0.2 |
| Xanthan Gum | 0.2 |
| Sodium Cocoyl Glutamate | 0.1 |
| Parfum | 0.1 |
| Glycerin | 0.2 |
| Argania Spinosa Kernel Oil | 0.1 |
| Titanium Dioxide | 0.1 |
| Hydroxypropyl Guar Hydroxypropyltrimonium Chloride | 0.1 |
| Lactic Acid | 0.1 |
| Hexyl Cinnamal | 0.1 |
| Sodium Hydroxide | 0.1 |
| Ascorbyl Palmitate | 0.1 |
| Tocopherol | 0.1 |

Example formulation 48: Natural Shampoo

| Ingredient | % |
|---|---|
| Aqua | ad 100 |
| Glycerin | 5.0 |
| Sodium Coco-sulfate | 5.0 |
| Lauryl Glucoside | 4.0 |
| Composition A | 4.0 |
| Betaine | 3.0 |
| Maris Sal | 2.0 |
| Camellia Oleifera Leaf Extract | 1.5 |
| Pisum Sativum Peptide | 1.5 |
| Oryza Sativa Extract | 1.0 |
| Pisum Sativum Seed Extract | 0.5 |
| Disodium Cocoyl Glutamate | 0.4 |
| Sodium Cocoyl Glutamate | 0.4 |
| PCA Glyceryl Oleate | 0.2 |
| Alcohols | 0.1 |
| Parfum | q.s. |

Example formulation 49: Anti-Hair Loss Tonic

| Ingredient | % |
|---|---|
| Aqua | ad 100 |
| Alcohol Denat. | 50.0 |
| PEG-40 Hydrogenated Castor Oil | 2.5 |
| Composition A | 1.5 |
| PEG-7 Glyceryl Cocoate | 2.0 |
| Sphinganine | 0.1 |
| Parfum, Preservative | q. s. |

Example formulation 50: Shower Oil

| Ingredient | % |
|---|---|
| Aqua | ad 100 |
| Magnesium Laureth Sulfate | 6.0 |
| PEG-7 Glyceryl Cocoate | 5.0 |
| Hydrogenated Starch Hydrolysate | 3.0 |
| Cocamidopropyl Betaine | 3.0 |
| Composition A | 3.0 |
| Decyl Glucoside | 1.0 |
| Prunus Amygdalus Dulcis (Sweet Almond) Oil | 1.0 |
| Olive Oil | 0.5 |
| PEG-7 Esters | 0.2 |
| Parfum | 0.1 |
| Polysorbate 20 | 0.1 |
| Sodium Benzoate | 0.2 |
| Citric Acid | 0.1 |
| PEG-6 Caprylic/Capric Glycerides | 0.1 |
| PEG-120 Methyl Glucose Dioleate | 0.1 |
| Disodium EDTA | 0.1 |

Example formulation 51: Shower Mousse

| Ingredient | % |
|---|---|
| Aqua | ad 100 |
| Sodium Lauroyl Glutamate | 15.0 |
| Isobutane | 6.0 |
| Cocamidopropyl Betaine | 4.0 |
| Glycerin | 3.0 |
| Composition A | 3.0 |
| Sodium Chloride | 2.6 |
| Sodium Lauroyl Glycinate | 1.5 |
| Propane | 1.0 |
| Butane | 0.6 |
| Citric Acid | 0.4 |
| lodopropynyl Butylcarbamate | 0.3 |
| Lauric Acid | 0.3 |
| Parfum | 0.1 |
| Polysorbate 20 | 0.1 |
| Sodium Benzoate | 0.1 |
| Sodium Hydroxide | 0.1 |
| Stearic Acid | 0.1 |
| Tetrasodium EDTA | 0.1 |

Example formulation 52: Shower Jelly

| Ingredient | % |
|---|---|
| Glycerin | 3.5 |
| Sodium Laureth Sulfate | 3.5 |
| Composition A | 3.0 |
| Propylene Glycol | 2.0 |
| Chondrus Crispus Extract | 2.0 |
| Pyrus Malus Juice | 1.0 |
| Vitis Vinifera Juice | 0.5 |
| Parfum | 0.2 |
| Butylphenyl Methylpropional | 0.1 |

Example formulation 53: Shower Jelly

| Ingredient | % |
|---|---|
| Aqua | ad 100 |
| Glycerin | 9.0 |
| Coco-Glucoside | 5.0 |
| Decyl Glucoside | 5.0 |
| Composition A | 2.0 |
| Chondrus crispus extract | 1.0 |
| Parfum | 0.2 |
| Citric Acid | 0.2 |
| Sodium Anisate | 0.1 |
| Sodium Levulinate | 0.1 |

Example formulation 54: Shower Scrub

| Ingredient | % |
|---|---|
| Aqua | ad 100 |
| Maris Sal | 15.0 |
| Sodium Laureth Sulfate | 9.0 |
| Composition A | 2.0 |
| Sodium Cocoamphoacetate | 1.0 |
| Betaine | 1.0 |
| Citrus Limon (Lemon) Peel Powder | 0.5 |
| Lactic Acid | ad pH 5.5 |
| Parfum, Dyes | q s. |

Example formulation 55: Cellulose Scrub

| Ingredient | % |
|---|---|
| Aqua | ad 100 |
| Composition A | 7.0 |
| Coco Glucoside | 6.0 |
| Glycerin | 5.0 |
| Glyceryl Stearate | 5.0 |
| Citrus Aurantium Dulcis Fruit Water | 4.5 |
| Helianthus Annuus Hybrid Oil | 4.0 |
| Cellulose | 2.0 |
| Jojoba Esters | 2.0 |
| Cetyl Alcohol | 2.0 |
| Simmondsia Chinensis (Jojoba) Seed Oil | 2.0 |
| Olea Europaea (Olive) Fruit Oil | 2.0 |
| Hydrogenated Castor Oil | 0.8 |
| Prunus Armeniaca (Apricot) Kernel Oil | 0.5 |
| Leontopodium Alpinum Extract | 0.1 |
| Xanthan Gum | 0.3 |
| Benzyl Alcohol | 0.2 |
| Parfum | 0.1 |
| Dehydroacetic Acid | 0.1 |
| Citric acid | ad pH 5.0 |
| Lactic acid | 0.1 |
| Sodium Hydroxide | 0.1 |
| Parfum, Dyes | q. s. |

Example formulation 56: Exfoliating Body Scrub

| Ingredient | % |
|---|---|
| Aqua | ad 100 |
| Coco-Glucoside | 7.0 |
| Alcohol | 6.0 |
| Glycerin | 5.0 |
| Glycine Soja Oil | 5.0 |
| Hydrated Silica | 4.0 |
| Xanthan Gum | 3.0 |
| Composition A | 3.0 |
| Disodium/Sodium Cocoyl Glutamate | 1.0 |
| Coffea Arabica (Coffee) Seed Powder | 1.0 |
| Cocos Nucifera (Coconut) Shell Powder | 1.0 |
| Citric Acid | ad pH 5.2 |
| Glyceryl Oleate | 0.5 |
| Parfum | 0.1 |
| Preservative, Dyes | q. s. |

Example formulation 57: Dry Shampoo

| Ingredient | % |
|---|---|
| Butane, Propane | 53.0 |
| Alcohol Denat. | 36.0 |
| Aluminum Starch Octenylsuccinate | 3.0 |
| Panthenol | 1.5 |
| Tocopherol | 1.0 |
| Cocos Nucifera Oil | 1.0 |
| Curcuma Longa (Turmeric) Root Extract | 0.6 |
| Silica | 0.5 |
| Sodium Bicarbonate | 0.5 |
| Charcoal Powder | 0.4 |
| Hydrolyzed Rice Protein | 0.4 |
| Aminopropyl Phenyl Trimethicone | 0.4 |
| Benzophenone-4 | 0.3 |
| Polysorbate 20 | 0.3 |
| Composition A | 0.3 |
| Cyclopentasiloxane | 0.2 |
| Cyclohexasiloxane | 0.2 |
| Glycerin | 0.2 |
| Aqua | 0.2 |
| Parfum | q.s. |

Example formulation 58: Dry Shampoo

| | % |
|---|---|
| Butane, Isobutane, Propane | 55.0 |
| Oryza Sativa (Rice) Starch | 25.0 |
| Alcohol denat. | 18.0 |
| Parfum | 1.0 |
| Composition A | 0.5 |
| Distearyldimonium Chloride | 0.3 |
| Cetrimonium Chloride | 0.2 |

### Shower Example Formulations

Example formulation 59: Shower Gel

| Ingredient | % |
|---|---|
| Aqua | ad 100 |
| Sodium Laureth Sulfate | 8.5 |
| Cocamidopropyl Betaine | 3.0 |
| Sodium Chloride | 2.7 |
| Composition A | 2.5 |
| Guar Gum | 0.5 |
| Polyquaternium-7 | 0.5 |
| Bisabolol | 0.2 |
| Trideceth-9 | 0.5 |
| Sodium Sulfate | 0.9 |
| Tetrasodium EDTA | 0.2 |
| Citric Acid | ad pH 5.5 |
| Sodium Benzoate | 0.7 |
| Benzyl Alcohol | 0.7 |
| Benzyl Salicylate | 0.1 |
| Geraniol | 0.2 |
| Hexyl Cinnamal | 0.1 |
| Limonene | 0.1 |
| Linalool | 0.1 |
| Dyes | q.s. |

Example formulation 60: Body Wash

| Ingredient | % |
|---|---|
| Water | ad 100 |
| Sodium Laureth Sulfate | 4.0 |
| Sodium Lauryl Sulfate | 4.0 |
| Sodium Chloride | 3.3 |
| Cocamidopropyl Betaine | 3.0 |
| Composition A | 3.0 |
| Sodium Benzoate | 1.5 |
| Citric Acid | ad pH 5.5 |
| Disodium EDTA | 0.5 |
| Zea Mays (Corn) Silk Extract | 0.5 |
| Methylchloroisothiazolinone | 0.1 |
| Methylisothiazolinone | 0.1 |
| Potassium Sorbate | 0.2 |
| Parfum, Dyes | q.s. |

Example formulation 61: Cool Down Shower Gel

| Ingredient | % |
|---|---|
| Aqua | ad 100 |
| Sodium Laureth Sulfate | 8.0 |
| Cocamidopropyl Betaine | 3.0 |
| Composition A | 3.0 |
| Glycerin | 2.7 |
| Acrylates Copolymer | 2.5 |
| Sodium Chloride | 2.2 |
| Phenoxyethanol | 1.5 |
| Taurine | 1.5 |
| Benzyl Alcohol | 1.0 |
| Sodium Hydroxide | 0.5 |
| PEG-40 Hydrogenated Castor Oil | 0.7 |
| Citric Acid | ad pH 5.0 |
| Mannitol | 0.7 |
| Benzophenone-4 | 0.3 |
| Ethylhexyl Salicylate | 0.5 |
| Cellulose | 0.5 |
| Linalool | 0.1 |
| Butyl Methoxydibenzoylmethane | 0.2 |
| Hydroxypropyl Methylcellulose | 0.2 |
| Dyes | q.s. |

Example formulation 62: Shower Gel

| Ingredient | % |
|---|---|
| Aqua | ad 100 |
| Cocamidopropyl Betaine | 5.0 |
| Sodium Myreth Sulfate | 4.5 |
| Sodium Laureth Sulfate | 4.0 |
| Composition A | 3.5 |
| PEG-7 Glyceryl Cocoate | 3.2 |
| Glycerin | 3.0 |
| Glyceryl Glucoside | 2.5 |
| Bisabolol | 2.0 |
| Chamomilla Recutita (Matricaria) Flower Extract | 2.2 |
| Sodium Chloride | 2.2 |
| PEG-40 Hydrogenated Castor Oil | 2.0 |
| Citric Acid | ad pH 5.0 |
| Glycolipids | 1.5 |
| PEG-90 Glyceryl Isostearate | 1.2 |
| PEG-200 Hydrogenated Glyceryl Palmate | 1.2 |
| Benzophenone-4 | 1.0 |
| Laureth-2 | 0.7 |
| Polyquaternium-7 | 0.5 |
| BHT | 0.2 |
| Sodium Benzoate | 0.2 |
| Methylparaben | 0.2 |
| Benzyl Alcohol | 0.2 |
| Citronellol | 0.1 |
| Limonene | 0.1 |
| Dyes | q.s. |

Example formulation 63: Shower Gel

| Ingredient | % |
|---|---|
| Aqua | ad 100 |
| Sodium Laureth Sulfate | 8.0 |
| Cocamidopropyl Betaine | 3.0 |
| Acrylates Copolymer | 3.0 |
| PEG-7 Glyceryl Cocoate | 2.7 |
| Composition A | 2.5 |
| Helianthus Annuus Seed Oil | 1.5 |
| Glycerin | 1.5 |
| Glycolipids | 1.5 |
| Sodium Chloride | 1.0 |
| PEG-40 Hydrogenated Castor Oil | 1.0 |
| Disodium Cocoyl Glutamate | 1.0 |
| Trisodium EDTA | 1.0 |
| Propylene Glycol | 1.0 |
| Microcrystalline Cellulose | 0.9 |
| Benzophenone-4 | 0.5 |
| Sodium Sulfate | 0.7 |
| Phenoxyethanol | 0.7 |
| Acacia Gum | |
| Propylparaben | 0.1 |
| Methylparaben | 0.1 |
| Benzyl Alcohol | 0.2 |
| Parfum, Dyes | q.s. |

Example formulation 64: Body Wash

| Ingredient | % |
|---|---|
| Aqua | ad 100 |
| Sodium Hydroxypropyl Starch Phosphate | 6.0 |
| Sodium Laureth Sulfate | 5.5 |
| Cocamidopropyl Betaine | 3.0 |
| Composition A | 3.0 |
| Lauric Acid | 2.5 |
| Petrolatum | 2.0 |
| Sodium Cocoyl Glycinate | 2.0 |
| Glycerin | 1.7 |
| Sodium Lauroyl Isethionate | 1.5 |
| Glycine Soja (Soybean) Oil | 1.0 |
| Helianthus Annuus (Sunflower) Oil | 1.0 |
| Sodium Chloride | 1.0 |
| Stearic Acid | 0.7 |
| Guar Hydroxypropyltrimonium Chloride | 0.3 |
| DMDM Hydantoin | 0.1 |
| Tallow Acid | 0.2 |
| Palmitic Acid | 0.1 |
| Sodium Isethionate | 0.2 |
| BHT | 0.1 |
| Tetrasodium EDTA | 0.2 |
| Methylisothiazolinone | 0.3 |
| Iodopropynyl Butylcarbamate | 0.1 |
| Titanium Dioxide | 0.1 |
| Parfum, Dyes | q.s. |

Example formulation 65: Anti-Aging Body Wash

| Ingredient | % |
|---|---|
| Aqua | ad 100 |
| Petrolatum | 5.0 |
| Mineral (Paraffinum Liquidum) Oil | 4.0 |
| Sodium Trideceth Sulfate | 3.5 |
| Sodium Lauryl Sulfate | 3.0 |
| Sodium Lauroamphoacetate | 3.0 |
| Sodium Chloride | 2.3 |
| Composition A | 2.0 |
| Trideceth-3 | 1.0 |
| Simmondsia Chinensis (Jojoba) Butter | 0.7 |
| Niacinamide | 0.1 |
| Panthenol | 0.3 |
| Soluble Collagen | 0.5 |
| Citric Acid | ad pH 5.5 |
| Disodium EDTA | 0.3 |
| Guar Hydroxypropyltrimonium Chloride | 0.3 |
| Methylchloroisothiazolinone | 0.1 |
| Methylisothiazolinone | 0.1 |
| PEG-90m | 0.7 |
| Sodium Benzoate | 0.3 |
| Sodium Hydroxide | 0.1 |
| Xanthan Gum | 0.5 |
| Calcium Alginate | .1 |
| Parfum, Dyes | q.s. |

Example formulation 66: Shower Gel

| Ingredient | % |
|---|---|
| Aqua | ad 100 |
| Sodium Laureth Sulfate | 9.0 |
| Cocamidopropyl Betaine | 3.0 |
| Composition A | 3.0 |
| Glycerin | 3.0 |
| Sodium Chloride | 2.8 |
| PEG-7 Glyceryl Cocoate | 2.5 |
| PEG-40 Hydrogenated Castor Oil | 2.5 |
| Citric Acid | ad pH 5.5 |
| Sodium Benzoate | 1.0 |
| Potassium Sorbate | 0.7 |
| Alcohol | 0.5 |
| Citronellol | 0.2 |
| Benzyl Alcohol | 0.7 |
| Limonene | 0.1 |
| Linalool | 0.2 |
| Geraniol | 0.2 |
| Hexyl Cinnamal | 0.1 |
| Ethylhexyl Salicylate | 0.1 |
| Octocrylene | 0.3 |
| Butyl Methoxydibenzoylmethane | 0.2 |
| Dyes | q.s. |

Example formulation 67: Shower Gel

| Ingredient | % |
|---|---|
| Aqua | ad 100 |
| Alcohol | 6.0 |
| Coco Glucoside | 6.0 |
| Glycerin | 3.0 |
| Composition A | 3.0 |
| Xanthan Gum | 1.5 |
| Disodium Cocoyl Glutamate | 2.0 |
| Citric Acid | ad pH 5.5 |
| Citrus Aurantifolia (Lime) Oil | 0.5 |
| Sodium Cocoyl Glutamate | 0.7 |
| Glyceryl Oleate | 0.3 |
| Sodium Lauroyl Lactylate | 0.8 |
| Limonene | 0.1 |
| Linalool | 0.1 |
| Dyes | q.s. |

Example formulation 68: Shower Gel for Men

| Ingredient | % |
|---|---|
| Aqua | ad 100 |
| Sodium Laureth Sulfate | 9.5 |
| Cocamidopropyl Betaine | 3.0 |
| Cocamide MEA | 3.0 |
| Composition A | 3.0 |
| Glycerin | 2.5 |
| PPG-12 | 2.0 |
| Sodium Chloride | 2.0 |
| Citric Acid | ad pH 5.5 |
| Sodium Benzoate | 1.0 |
| Phenoxyethanol | 0.7 |
| Benzyl Alcohol | 0.7 |
| Butylphenyl Methylpropional | 0.1 |
| Citronellol | 0.1 |
| Geraniol | 0.2 |
| Hexyl Cinnamal | 0.1 |
| Limonene | 0.1 |
| Linalool | 0.1 |
| Dyes | q.s. |

Example formulation 69: Moisturising Shower Cream

| Ingredient | % |
|---|---|
| Aqua | ad 100 |
| Sodium Laureth Sulfate | 8.5 |
| Cocamidopropyl Betaine | 3.0 |
| Glycerin | 2.5 |
| Composition A | 2.0 |
| Sodium Chloride | 1.7 |
| Cocamide MEA | 1.5 |
| Styrene/Acrylates Copolymer | 1.5 |
| Sodium Salicylate | 1.0 |
| Sodium Benzoate | 1.0 |
| Polyquaternium-7 | 1.0 |
| Citric Acid | ad pH 5.0 |
| Tetrasodium EDTA | 1.0 |
| Glycol Distearate | 0.9 |
| Laureth-4 | 0.5 |
| Magnolia Officinalis Flower Extract | 0.2 |
| Butyrospermum Parkii Butter Extract | 0.2 |
| Prunus Amygdalus Dulcis (Sweet Almond) Extract | 0.1 |
| Tapioca Starch polymethylsilsesquioxane | 0.5 |
| Benzyl Benzoate | 0.1 |
| Cinnamyl Alcohol | 0.1 |
| Hexyl Cinnamal | 0.1 |
| Parfum, Dyes | q.s. |

Example formulation 70: Shower Cream

| | |
|---|---|
| Aqua | ad 100 |
| Ammonium Lauryl Sulfate | 6.0 |
| Coco Glucoside | 3.5 |
| Cocamidopropyl Betaine | 2.0 |
| Sodium Cocoamphoacetate | 2.0 |
| Composition A | 1.5 |
| Argania Spinosa Kernel Oil | 1.0 |
| Butyrospermum Parkii Butter | 1.0 |
| Glycerin | 1.0 |
| Polyquaternium-7 | 1.0 |
| Styrene/Acrylates Copolymer | 1.0 |
| Gelatin Crosspolymer | 0.5 |
| Sodium Chloride | 0.5 |
| Citric Acid | ad pH 5.0 |
| Sodium Benzoate | 0.3 |
| Sodium Salicylate | 0.1 |
| Linalool | 0.1 |
| Dyes | q.s. |

Example formulation 71: Shower Cream

| Ingredient | % |
|---|---|
| Aqua | ad 100 |
| Sodium Laureth Sulfate | 7.0 |
| Cocamidopropyl Betaine | 3.0 |
| Composition A | 2.0 |
| Sodium Chloride | 1.9 |
| Polyquaternium-7 | 1.5 |
| Cocamide MEA | 1.5 |
| PEG-7 Glyceryl Cocoate | 1.0 |
| Laureth-10 | 1.0 |
| Glycol Distearate | 0.8 |
| Citric Acid | ad pH 5.0 |
| Linalool | 0.1 |
| Limonene | 0.1 |
| Benzyl Salicylate | 0.1 |
| Butylphenyl Methylpropional | 0.1 |
| alpha-Isomethyl Ionone | 0.1 |
| Hexyl Cinnamal | 0.1 |
| Citronellol | 0.1 |
| Sodium Benzoate | 0.7 |
| Sodium Salicylate | 0.1 |
| Dyes | q.s. |

Example formulation 72: Shower Cream

| Ingredient | % |
|---|---|
| Aqua | ad 100 |
| Glycerin | 7.5 |
| Glycine Soja Oil | 3.0 |
| Lauryl Glucoside | 3.0 |
| Sodium Coco Sulfate | 3.0 |
| Composition A | 2.5 |
| Alcohol | 1.5 |
| Xanthan Gum | 1.5 |
| Butyrospermum Parkii Butter | 1.2 |
| Sodium Cetearyl Sulfate | 1.0 |
| Sodium Cocoyl Glutamate | 1.0 |
| Disodium Cocoyl Glutamate | 1.0 |
| Tocopherol | 0.1 |
| Helianthus Annuus Seed Oil | 0.3 |
| Limonene | 0.1 |
| Benzyl Salicylate | 0.1 |
| Linalool | 0.1 |
| Dyes | q.s. |

Example formulation 73: Shower Gel

| Ingredient | % |
|---|---|
| Aqua | ad 100 |
| Sodium Coco Sulfate | 5.0 |
| Glycerin | 5.0 |
| Lauryl Glucoside | 4.0 |
| Sodium Lactate | 2.5 |
| Composition A | 2.0 |
| Sodium Cocoyl Glutamate | 2.0 |
| Disodium Cocoyl Glutamate | 2.0 |
| Alcohol | 1.0 |
| Prunus Cerasus Fruit Extract | 1.0 |
| Limonene | 0.1 |
| Coumarin | 0.2 |
| Linalool | 0.1 |
| Citral | 0.1 |
| Dyes | q.s. |

Example formulation 74: Shower Gel

| Ingredient | % |
|---|---|
| Aqua | ad 100 |
| Alcohol | 5.0 |
| Coco-Glucoside | 5.0 |
| Glycerin | 5.0 |
| Composition A | 2.5 |
| Xanthan Gum | 2.5 |
| Disodium Cocoyl Glutamate | 2.0 |
| Citric Acid | ad pH 5.0 |
| Citrus Aurantifolia (Lime) Oil | 1.0 |
| Sodium Cocoyl Glutamate | 1.0 |
| Glyceryl Oleate | 1.0 |
| Sodium Lauroyl Lactylate | 0.7 |
| Limonene | 0.1 |
| Citral | 0.1 |
| Linalool | 0.1 |
| Dyes | q.s. |

Example formulation 75: Oil Shower

| Ingredient | % |
|---|---|
| Aqua | ad 100 |
| Sodium Laureth Sulfate | 8.0 |
| Cocamidopropyl Betaine | 3.0 |
| Composition A | 2.0 |
| Acrylates Copolymer | 2.0 |
| Glycolipids | 2.0 |
| Tocopheryl Acetate | 0.5 |
| PEG-7 Glyceryl Cocoate | 0.5 |
| Disodium EDTA | 0.4 |
| Benzophenone-4 | 0.3 |
| PEG-40 Hydrogenated Castor Oil | 0.7 |
| Cellulose | 0.7 |
| Hydroxypropyl Methylcellulose | 0.7 |
| Sodium Chloride | 0.9 |
| Ethylhexylglycerin | 0.2 |
| Phenoxyethanol | 0.7 |
| Methylisothiazolinone | 0.3 |
| Sodium Benzoate | 0.3 |
| Sodium Hydroxide | 0.3 |
| Citric Acid | ad pH 5.5 |
| Parfum, Dyes | q.s. |

Example formulation 76: Shower Gel

| Ingredient | % |
|---|---|
| Aqua | ad 100 |
| Ammonium Lauryl Sulfate | 6.0 |
| Coco-Betaine | 3.0 |
| Composition A | 3.0 |
| Lauroyl/Myristoyl Methyl Glucamide | 1.5 |
| Citric Acid | ad pH 5.0 |
| Benzyl Alcohol, Glycerin, Benzoic Acid, Sorbic Acid | 1.0 |
| Parfum | 0.8 |
| Aloe Vera Leaf Extract | 0.1 |

Example formulation 77: Showergel

| Ingredient | % |
|---|---|
| Aqua | ad 100 |
| Composition A | 7.0 |
| Sesamum Indicum (Sesame) Seed Oil | 4.0 |
| Glycolipid | 3.0 |
| Sucrose. Alcohol | 2.0 |
| Amino Acids | 1.0 |
| Glycerin | 1.0 |
| Carrageenan | 0.3 |
| Lavandula Officinalis Flower Oil | 0.3 |
| Xanthan Gum | 0.3 |
| Lactic Acid | 0.1 |

Example formulation 78: Shower Scrub

| Ingredient | % |
|---|---|
| Aqua | ad 100 |
| Maris Sal | 15.0 |
| Sodium Laureth Sulfate | 9.0 |
| Composition A | 2.0 |
| Sodium Cocoamphoacetate | 1.0 |
| Betaine | 1.0 |
| Citrus Limon (Lemon) Peel Powder | 0.5 |
| Lactic Acid | ad pH 5.5 |
| Parfum, Dyes | q s. |

Example formulation 79: Cellulose Scrub

| Ingredient | % |
|---|---|
| Aqua | ad 100 |
| Composition A | 7.0 |
| Coco Glucoside | 6.0 |
| Glycerin | 5.0 |
| Glyceryl Stearate | 5.0 |
| Citrus Aurantium Dulcis Fruit Water | 4.5 |
| Helianthus Annuus Hybrid Oil | 4.0 |
| Cellulose | 2.0 |
| Jojoba Esters | 2.0 |
| Cetyl Alcohol | 2.0 |
| Simmondsia Chinensis (Jojoba) Seed Oil | 2.0 |
| Olea Europaea (Olive) Fruit Oil | 2.0 |
| Hydrogenated Castor Oil | 0.8 |
| Prunus Armeniaca (Apricot) Kernel Oil | 0.5 |
| Leontopodium Alpinum Extract | 0.1 |
| Xanthan Gum | 0.3 |
| Benzyl Alcohol | 0.2 |
| Parfum | 0.1 |
| Dehydroacetic Acid | 0.1 |
| Citric acid | ad pH 5.0 |
| Lactic acid | 0.1 |
| Sodium Hydroxide | 0.1 |
| Parfum, Dyes | q.s. |

Example formulation 80: Exfoliating Body Scrub

| Ingredient | % |
|---|---|
| Aqua | ad 100 |
| Coco-Glucoside | 7.0 |
| Alcohol | 6.0 |
| Glycerin | 5.0 |
| Glycine Soja Oil | 5.0 |
| Hydrated Silica | 4.0 |
| Xanthan Gum | 3.0 |
| Composition A | 3.0 |
| Disodium/Sodium Cocoyl Glutamate | 1.0 |
| Coffea Arabica (Coffee) Seed Powder | 1.0 |
| Cocos Nucifera (Coconut) Shell Powder | 1.0 |
| Citric Acid | ad pH 5.2 |
| Glyceryl Oleate | 0.5 |
| Parfum | 0.1 |
| Preservative, Dyes | q.s. |

Example formulation 81: Washing Powder

| Ingredient | % |
|---|---|
| Talc | 60.0 |
| Sodium Cocoyl Isethionate | 15.0 |
| Sodium C14-16 Olefin Sulfonate | 10.0 |
| Sodium Lauroyl Glutamate | 4.0 |
| Composition A | 3.0 |
| Potassium Laurate | 2.0 |
| Sodium Myristoyl Glutamate | 1.5 |
| Chondrus Crispus | 1.5 |
| Isostearyl Alcohol | 1.0 |
| Glycerin | 1.0 |
| BHT | 0.4 |
| Butylene Glycol | 0.3 |
| Protease | 0.2 |
| Salicylic Acid | 0.1 |

Example formulation 82: Washing Powder

| Ingredient | % |
|---|---|
| Hydrates Silica | ad 100 |
| Zea Mays Starch | 30.0 |
| Sodium Cocoyl Isethionate | 6.0 |
| Amylopektin | 5.0 |
| Sodium Lauryl Sulfate | 2.5 |
| Sodium Bicarbonate | 2.0 |
| Citric Acid | 1.5 |
| Sodium Lauroyl Glutamate | 1.0 |
| Sodium Palmitate | 1.0 |
| Composition A | 1.0 |
| Diglycerin | 0.9 |
| Sodium Lauroyl Aspartate | 0.8 |
| Titanium Dioxide | 0.5 |
| Maltodextrin | 0.5 |
| Allantoin | 0.4 |
| Aqua | 0.3 |
| Butylene Glycol | 0.3 |
| Aloe Vera Leaf Extract | 0.1 |
| Citrus Limon (Lemon) Peel Powder | 0.1 |
| Parfum, Preservatives, Dyes | q.s. |

Example formulation 83: Powder Cleanser

| Ingredient | % |
|---|---|
| Hydrated Silica | ad 100 |
| Tapioca Flour | 10.0 |
| Capryl/Capramidopropyl Betaine | 5.0 |
| Sodium Cocoamphoacetate | 5.0 |
| Helianthus Annuus (Sunflower) Seed Oil | 4.0 |
| Oleyl Erucate | 2.0 |
| Sucrose Cocoate | 2.0 |
| Composition A | 2.0 |
| Coconut Acid | 1.0 |
| Citric Acid | ad pH 6.0 |
| Preservatives, parfum | q.s. |

Example formulation 84: Powder Cleanser

| Ingredient | % |
|---|---|
| Talc | ad 100 |
| Distarch Phosphate | 10.0 |
| Disodium Lauryl Sulfosuccinate | 8.0 |
| Magnesium Aluminum Silicate | 5.0 |
| Oryza Sativa Powder | 5.0 |
| Sodium Lauroyl Glutamate | 2.0 |
| Composition A | 2.0 |
| Glucose | 2.0 |
| Magnesium Oxide | 2.0 |
| Maltodextrin | 2.0 |
| Titanium Dioxide | 1.0 |
| Pumice | 0.5 |
| Chlorella Vulgaris Powder | 0.1 |

### Soap Example Formulations

Example formulation 85: Gentle Liquid Soap

| Ingredient | % |
|---|---|
| Aqua | ad 100 |
| Sodium Laureth Sulfate | 8.5 |
| Cocamidopropyl Betaine | 3.0 |
| Composition A | 2.5 |
| PEG-7 Glyceryl Cocoate | 2.5 |
| Cocamide DEA | 2.5 |
| Glycerin | 2.0 |
| Sorbitan Sesquicaprylate | 1.5 |
| C12-15 Pareth-12 | 1.2 |
| Sodium Benzoate | 1.0 |
| Potassium Sorbate | 1.0 |
| Citric Acid | ad pH 5.5 |
| PEG-200 Hydrogenated Glyceryl Palmate | 1.0 |
| Sodium Lactate | 1.0 |
| Sodium Chloride | 0.7 |
| Tetrasodium Glutamate Diacetate | 0.2 |
| Linalool | 0.1 |
| Limonene | 0.1 |
| Citronellol | 0.1 |
| Dyes | q.s. |

Example formulation 86: Soap

| Ingredient | % |
|---|---|
| Aqua | ad 100 |
| Cocamidopropyl Betaine | 5.0 |
| Glycolipids | 2.5 |
| Composition A | 4.0 |
| Glycerin | 2.7 |
| PEG-7 Glyceryl Cocoate | 2.5 |
| Simmondsia Chinensis (Jojoba) Seed Oil | 2.5 |
| Glyceryl Glucoside | 2.3 |
| Mel | 2.0 |
| Sodium Laureth Sulfate | 3.0 |
| Sodium Chloride | 2.1 |
| Citric Acid | ad pH 5.0 |
| PEG-40 Hydrogenated Castor Oil | 1.8 |
| PEG-120 Methyl Glucose Dioleate | 1.7 |
| Trisodium EDTA | 1.0 |
| Glycol Distearate | 1.0 |
| Polyquaternium-10 | 0.3 |
| Laureth-10 | 0.7 |
| Benzophenone-4 | 0.5 |
| Cocamide MEA | 0.8 |
| Maltodextrin | 0.5 |
| Sodium Benzoate | 0.7 |
| Sodium Salicylate | 0.6 |
| Benzoic Acid | 0.4 |
| Linalool | 0.1 |
| Parfum, Dyes | q.s. |

Example formulation 87: Caring Liquid Soap

| Ingredient | % |
|---|---|
| Aqua | ad 100 |
| Aloe Barbadensis Leaf Juice | 5.0 |
| Ammonium Lauryl Sulfate | 5.0 |
| Coco Glucoside | 4.0 |
| Cocamidopropyl Betaine | 4.0 |
| Sodium Cocoamphoacetate | 1.5 |
| Composition A | 1.5 |
| Glycerin | 1.5 |
| Sodium Chloride | 1.5 |
| Citric Acid | ad pH 5.5 |
| Sodium Benzoate | 1.0 |
| Sodium Salicylate | 0.5 |
| Methylisothiazolinone | 0.3 |
| Linalool | 0.1 |
| Butylphenyl Methylpropional | 0.2 |
| Limonene | 0.1 |
| Dyes | q.s. |

Example formulation 88: Cream Soap

| Ingredient | % |
|---|---|
| Aqua | ad 100 |
| Alcohol | 5.0 |
| Coco Glucoside | 5.0 |
| Glycerin | 5.0 |
| Composition A | 2.5 |
| Disodium Cocoyl Glutamate | 2.0 |
| Xanthan Gum | 1.5 |
| Citric Acid | ad pH 5.5 |
| Malva Sylvestris Leaf Extract | 1.0 |
| Sodium Lauroyl Lactylate | 1.0 |
| Glyceryl Oleate | 1.0 |
| Sodium Cocoyl Glutamate | 0.7 |
| Linalool | 0.1 |
| Limonene | 0.1 |
| Dyes | q.s. |

Example formulation 89: Liquid Soap

| Ingredient | % |
|---|---|
| Aqua | ad 100 |
| Sodium Laureth Sulfate | 5.0 |
| Sodium Chloride | 3.5 |
| Cocamidopropyl Betaine | 3.5 |
| Coco Glucoside | 3.0 |
| Composition A | 2.0 |
| Glyceryl Oleate | 1.0 |
| Hibiscus Sabdariffa Flower Extract | 1.0 |
| Alcohol | 1.0 |
| Sodium Lactate | 1.0 |
| Citric Acid | ad pH 5.0 |
| Sodium Benzoate | 0.7 |
| Potassium Sorbate | 0.1 |
| Parfum, Dyes | q.s. |

Example formulation 90: Liquid Soap

| Ingredient | % |
|---|---|
| Aqua | ad 100 |
| Glycerin | 7.0 |
| Alcohol | 4.0 |
| Sodium Coco Sulfate | 3.0 |
| Lauryl Glucoside | 2.5 |
| Composition A | 2.0 |
| Xanthan Gum | 1.5 |
| Mangifera Indica (Mango) Fruit Extract | 0.7 |
| Limonene | 0.1 |
| Linalool | 0.1 |
| Dyes | q.s. |

Example formulation 91: Liquid Soap

| Ingredient | % |
|---|---|
| Aqua | ad 100 |
| Sodium Lauryl Sulfate | 6.0 |
| Cocamidopropyl Betaine | 3.0 |
| Composition A | 3.0 |
| Triethylcitrate; Caprylyl Glycol; Benzoic Acid | 1.0 |
| Lactic Acid | ad pH 5.0 |
| Chamomilla Recutita Extract | 0.5 |
| Sodium Chloride | 0.9 |
| Sodium Lactate | 0.7 |
| Styrene/Acrylates Copolymer | 0.5 |
| Glycerin | 0.5 |
| Polyquaternium-7 | 0.3 |
| Hexyl Cinnamal | 0.1 |
| Sorbitol | 0.2 |
| Trisodium Ethylenediamine Disuccinate | 0.2 |
| Propylene Glycol | 0.3 |
| PEG-40 Hydrogenated Castor Oil | 0.9 |
| 1,2-Hexandiol | 0.5 |
| Parfum, Dyes | q.s. |

Example formulation 92: Cream Soap

| Ingredient | % |
|---|---|
| Aqua | ad 100 |
| Sodium Laureth Sulfate | 8.0 |
| Cocamidopropyl Betaine | 3.0 |
| Sodium Chloride | 2.6 |
| Composition A | 2.5 |
| Polyquaternium-7 | 0.4 |
| Glycerin | 0.9 |
| Glycol Distearate | 0.7 |
| Laureth-4 | 0.5 |
| Laureth-2 | 0.8 |
| PEG-55 Propylene Glycol Oleate | 0.4 |
| Propylene Glycol | 0.5 |
| Citric Acid | ad pH 5.5 |
| Hexyl Cinnamal | 0.1 |
| Benzyl Salicylate | 0.1 |
| Linalool | 0.1 |
| Limonene | 0.1 |
| Butylphenyl Methylpropional | 0.1 |
| Sodium Benzoate | 0.1 |
| Dyes | q.s. |

Example formulation 93: Jelly Soap

| Ingredient | % |
|---|---|
| Glycerin | 50.0 |
| Decyl Glucoside | 5.0 |
| Glycolipids | 3.0 |
| Composition A | 2.5 |
| Chondrus Crispus Powder | 2.0 |
| Methylpropanediol | 1.0 |
| Caprylyl Glycol | 1.0 |
| Phenylpropanol | 0.5 |
| Parfum | 0.2 |
| Helianthus Annuus (Sunflower) Seed Oil | 0.2 |
| Hydrogenated Coconut Oil | 0.2 |
| Olea Europea (Olive) Fruti Oil | 0.2 |
| Oryza Sativa (Rice) Bran Oil | 0.2 |
| Vitis Vinifera (Grape) Seed Oil | 0.1 |
| Prunus Amygdalus Dulcis (Sweet Almond) Oil | 0.1 |
| Aqua | ad 100 |
| Sodium Benzoate | 0.1 |
| Citric Acid | 0.1 |

Example formulation 94: Soap

| Ingredient | % |
|---|---|
| Talc | 65.0 |
| Composition A | 10.0 |
| Corn (Zea Mays) Flour | 9.0 |
| Glycerin | 7.0 |
| Sodium Laureth Sulfate | 4.5 |
| Citrus Aurantium Bergamia Fruit Oil | 3.5 |
| Benzyl Benzoate | 0.5 |
| Butylphenyl Methylpropional | 0.2 |
| Parfum | 0.2 |

Example formulation 95: Soap

| Ingredient | % |
|---|---|
| Aqua | ad 100 |
| Composition A | 6.0 |
| Glycolipids | 5.0 |
| Propylene Glycol | 5.0 |
| Helianthus Annuus Seed Oil | 4.0 |
| Cocos Nucifera Oil | 4.0 |
| Titanium Dioxide | 0.2 |
| Sodium Hydroxide | 0.2 |
| Sodium Coco-sulfate | 0.2 |
| Parfum | 0.1 |
| Sodium Chloride | 0.1 |
| L-limonene | 0.1 |

Example formulation 96: Painting Soap

| Ingredient | % |
|---|---|
| Aqua | ad 100 |
| Mica | 7.0 |
| Glycine Soja Oil | 5.0 |
| Glycerin | 4.0 |
| Alcohol | 3.0 |
| Cocamidopropyl Betaine | 3.0 |
| Composition A | 2.0 |
| Cetearyl Alcohol | 1.5 |
| Myristyl Alcohol | 1.0 |
| Glyceryl Stearate Citrate | 1.0 |
| Parfum | 0.4 |
| Xanthan Gum | 0.3 |
| Helianthus Annuus Seed Oil | 0.2 |
| Simmondsia Chinensis Seed Oil | 0.2 |
| Rubus Idaeus Fruit Extract | 0.2 |
| Tocopherol | 0.1 |

Example formulation 97: Painting Soap

| Ingredient | % |
|---|---|
| Aqua | ad 100 |
| Glycerin | 10.0 |
| Zea Starch | 9.0 |
| Lauryl Glucoside | 4.0 |
| Methylpropanediol | 3.0 |
| Stearyl Alcohol | 3.0 |
| Cocamidopropyl Betaine | 2.0 |
| Composition A | 2.0 |
| Glycine Soja Oil | 1.0 |
| Tocopherol | 0.5 |
| Coco-Glucoside | 0.4 |
| Panthenol | 0.2 |
| Glycerin Oleate | 0.2 |
| Caprylyl Glycol | 0.2 |
| Phenlypropanol | 0.2 |
| Sodium Hydroxide | 0.1 |
| Sodium Chloride | 0.1 |
| Xanthan Gum | 0.1 |
| Citric Acid | 0.1 |
| Parfum | 0.1 |
| Beta Carotene | 0.1 |
| Dyes | q.s. |

### Bath Example Formulations

Example formulation 98: Bath

| Ingredient | % |
|---|---|
| Aqua | ad 100 |
| PEG-80 Sorbitan Laurate | 4.0 |
| Cocamidopropyl Betaine | 4.0 |
| Sodium Laureth Sulfate | 4.0 |
| Composition A | 2.0 |
| Polysorbate 20 | 2.0 |
| Sodium Chloride | 1.5 |
| Citric Acid | ad pH 5.0 |
| Sodium Lauroamphoacetate | 1.0 |
| PEG-150 Distearate | 1.0 |
| Disodium Lauroamphodiacetate | 1.0 |
| Sodium Benzoate | 0.5 |
| Parfum, Dyes | q.s. |

Example formulation 99: Creme Oil Bath

| Ingredient | % |
|---|---|
| Aqua | ad 100 |
| Sodium Laureth Sulfate | 5.0 |
| Cocamidopropyl Betaine | 3.0 |
| Decyl Glucoside | 2.8 |
| Composition A | 2.5 |
| Glycerin | 2.5 |
| Glyceryl Glucoside | 2.0 |
| Diamond Powder | 2.0 |
| Helianthus Annuus (Sunflower) Seed Oil | 1.7 |
| Sodium Chloride | 1.5 |
| Glycol Distearate | 1.5 |
| PEG-40 Hydrogenated Castor Oil | 1.5 |
| Citric Acid | ad pH 5.5 |
| PEG-90 Glyceryl Isostearate | 1.2 |
| Laureth-4 | 1.0 |
| PEG-200 Hydrogenated Glyceryl Palmate | 1.0 |
| PEG-7 Glyceryl Cocoate | 1.0 |
| Benzophenone-4 | 0.9 |
| Laureth-2 | 0.7 |
| Sodium Benzoate | 0.7 |
| Butylphenyl Methylpropional | 0.1 |
| Linalool | 0.1 |
| Limonene | 0.1 |
| alpha-Isomethyl lonone | 0.1 |
| Dyes | q.s. |

Example formulation 100: Relaxing Good Sleep Bath

| Ingredient | % |
|---|---|
| Aqua | ad 100 |
| Cocamidopropyl Betaine | 3.0 |
| Coco Glucoside | 2.5 |
| Sodium Chloride | 2.5 |
| Disodium Lauryl Sulfosuccinate | 2.0 |
| Composition A | 2.0 |
| Glycerin | 1.7 |
| Sodium PCA | 0.5 |
| Glyceryl Oleate | 0.4 |
| Sodium Hydroxide | 0.3 |
| Citric Acid | ad pH 5.5 |
| Linalool | 0.1 |
| Limonene | 0.1 |
| Geraniol | 0.1 |
| Dyes | q.s. |

Example formulation 51: Pampering Oil Bath

| Ingredient | % |
|---|---|
| Aqua | ad 100 |
| Glycine Soja Oil | 20.0 |
| Composition A | 5.0 |
| Polyglyceryl-3 Palmitate | 4.5 |
| Glyceryl Caprylate | 1.5 |
| Simmondsia Chinensis Oil | 1.5 |
| Prunus Amygdalus Dulcis (Sweet Almond) Oil | 1.0 |
| Triticum Vulgare Germ Oil | 1.0 |
| Tocopherol | 0.2 |
| Limonene | 0.1 |
| Linalool | 0.1 |
| p-Anisic Acid | 0.1 |
| Dyes | q.s. |

Example formulation 101: Aroma Bath

| Ingredient | % |
|---|---|
| Aqua | ad 100 |
| Alcohol | 5.0 |
| Coco Glucoside | 5.0 |
| Sodium Coco Sulfate | 4.0 |
| Composition A | 2.5 |
| Xanthan Gum | 1.9 |
| Glycerin | 1.8 |
| Sodium Cocoyl Glutamate | 1.5 |
| Disodium Cocoyl Glutamate | 1.5 |
| Melissa Officinalis Extract | 1.0 |
| Lavandula Angustifolia Oil | 1.0 |
| Linalool | 0.1 |
| Limonene | 0.1 |
| Geraniol | 0.1 |
| Dyes | q.s. |

Example formulation 102: Bath Bomb

| Ingredient | % |
|---|---|
| Sodium Bicarbonate | 70.0 |
| Citric Acid | 8.0 |
| Citrus Nobilis Oil | 6.0 |
| Potassium Tartrate | 6.0 |
| Sodium Laureth Sulfate | 4.0 |
| Composition A | 4.0 |
| Betaine | 1.5 |
| Parfum | 0.5 |
| Dyes | q.s. |

Example formulation 103: Bath Bomb

| Ingredient | % |
|---|---|
| Sodium Bicarbonate | 67.0 |
| Citric Acid | 9.0 |
| Zea Mays Starch | 8.0 |
| Butyrospermum Parkii Butter | 5.0 |
| Sodium Lauryl Sulfoacetate | 4.0 |
| Composition A | 4.0 |
| Theobroma Cacao Seed Butter | 2.0 |
| Theobroma Cacao (Cocoa) Fruit Powder | 1.0 |
| Parfum, Dyes | q.s. |

Example formulation 104: Bath Salt

| Ingredient | % |
|---|---|
| Maris Sal | ad 100 |
| Parfum | 2.0 |
| Glycolipids | 1.0 |
| Composition A | 1.0 |
| Silica | 0.5 |
| Simmondsia Chinensis Seed Oil | 0.1 |
| Preservative, Dyes | q.s. |

Example formulation 105: Bath Salt

| Ingredient | % |
|---|---|
| Maris Sal | ad 100 |
| Rosmarinus Officinalis Leaf Oil | 3.0 |
| Parfum | 1.5 |
| Polysorbate 20 | 1.5 |
| Composition A | 1.5 |
| Preservative, Dyes | q.s. |

x-In-1 Example Formulations

Example formulation 106: Shower Gel and Shampoo

| Ingredient | % |
|---|---|
| Aqua | ad 100 |
| Coco Glucoside | 5.0 |
| Sodium Coco Sulfate | 3.0 |
| Cocamidopropyl Hydroxysultaine | 2.5 |
| Composition A | 2.0 |
| Lauryl Glucoside | 2.0 |
| Glyceryl Oleate | 1.8 |
| Inulin | 1.5 |
| Lecithin | 1.5 |
| Polyquaternium-39 | 1.5 |
| Citric Acid | ad pH 5.0 |
| Styrene/Acrylates Copolymer | 1.0 |
| Sodium Chloride | 1.0 |
| Denatonium Benzoate | 0.5 |
| Hydrogenated Palm Glycerides Citrate | 0.7 |
| Tocopherol | 0.3 |
| Sodium Benzoate | 0.7 |
| Phenoxyethanol | 0.3 |
| Parfum, Dyes | q.s. |

Example formulation 107: Shower, Shampoo and Shave

| Ingredient | % |
|---|---|
| Aqua | ad 100 |
| Cocamidopropyl Betaine | 5.0 |
| Sodium Laureth Sulfate | 4.0 |
| PEG-7 Glyceryl Cocoate | 4.0 |
| Sodium Chloride | 3.5 |
| Composition A | 2.0 |
| PEG-3 Distearate | 3.0 |
| Glycerin | 2.5 |
| Glyceryl Glucoside | 1.2 |
| Helianthus Annuus (Sunflower) Seed Oil | 1.0 |
| Persea Gratissima (Avocado) Oil | 1.0 |
| Polyquaternium-7 | 1.0 |
| PEG-90m | 1.0 |
| PEG-40 Hydrogenated Castor Oil | 1.0 |
| Citric Acid | ad pH 5.0 |
| Benzophenone-4 | 0.5 |
| PEG-200 Hydrogenated Glyceryl Palmate | 0.5 |
| Silica | 0.7 |
| Sodium Benzoate | 0.6 |
| Methylparaben | 0.4 |
| Propylparaben | 0.4 |
| Limonene | 0.1 |
| Butylphenyl Methylpropional | 0.1 |
| Benzyl Alcohol | 0.1 |
| Dyes | q.s. |

Example formulation 108: 2-in-1 Body Wash and Shampoo

| Ingredient | % |
|---|---|
| Aqua | ad 100 |
| Sodium Laureth Sulfate | 8.5 |
| Glycerin | 2.5 |
| Sodium Cocoyl Isethionate | 2.5 |
| Ammonium Lauryl Sulfate | 2.5 |
| Lauryl Betaine | 2.5 |
| Lauryl Glucoside | 1.5 |
| Sodium Chloride | 1.0 |
| Composition A | 1.0 |
| Polyquaternium-39 | 0.5 |
| Polyquaternium-7 | 0.5 |
| Panthenol | 0.1 |
| Citric Acid | ad pH 5.5 |
| Sodium Citrate | 0.2 |
| Propylene Glycol | 0.3 |
| PEG-55 Propylene Glycol Oleate | 0.7 |
| Styrene/Acrylates Copolymer | 0.2 |
| Phenoxyethanol | 0.7 |
| Sodium Benzoate | 0.3 |
| Tetrasodium EDTA | 0.1 |
| Parfum, Dyes | q.s. |

Example formulation 109: 2-in-1 Bubble Bath and Wash

| Ingredient | % |
|---|---|
| Aqua | ad 100 |
| Cocamidopropyl Betaine | 4.0 |
| Glycerin | 4.0 |
| Sodium Lauroamphodiacetate | 2.0 |
| Sodium Chloride | 2.0 |
| Coco Glucoside | 1.5 |
| Hydroxypropyl Starch Phosphate | 1.0 |
| Disodium Lauroamphodiacetate | 1.0 |
| Composition A | 1.0 |
| Lauric Acid | 1.0 |
| Acrylates/C10-30 Alkyl Acrylate Crosspolymer | 1.0 |
| Polyquaternium-7 | 1.0 |
| Styrene/Acrylates Copolymer | 0.5 |
| Citric Acid | ad pH 5.5 |
| Sodium Hydroxide | 0.3 |
| Sodium Benzoate | 0.2 |
| Benzoic Acid | 0.1 |
| Parfum, Dyes | q.s. |

Example formulation 110: Body and Face Wash

| Ingredient | % |
|---|---|
| Aqua | ad 100 |
| Sodium Laureth Sulfate | 4.5 |
| Petrolatum | 3.0 |
| Acrylates Copolymer | 2.5 |
| Cocamide MEA | 2.5 |
| Composition A | 2.0 |
| PPG-9 | 2.0 |
| Guar hydroxypropyltrimonium chloride | 0.2 |
| Menthol | 0.2 |
| DMDM Hydantoin | 0.1 |
| Tetrasodium EDTA | 0.2 |
| Methylisothiazolinone | 0.1 |
| BHT | 0.1 |
| Parfum, Dyes | q.s. |

Example formulation 111: Hair and Body Shampoo

| Ingredient | % |
|---|---|
| Aqua | ad 100 |
| Sodium Laureth Sulfate | 10.0 |
| Sodium Chloride | 4.0 |
| Glycerin | 3.5 |
| Coco-Betaine | 2.0 |
| Composition A | 2.0 |
| Niacinamide | 0.1 |
| Saccharum Officinarum (Sugar Cane) Extract | 1.0 |
| Sodium Benzoate | 1.0 |
| Sodium Hydroxide | 0.3 |
| PPG-5-Ceteth-20 | 1.0 |
| Polyquaternium-10 | 0.7 |
| Salicylic Acid | 0.5 |
| Limonene | 0.1 |
| Linalool | 0.2 |
| Benzyl Salicylate | 0.1 |
| Pyrus Malus (Apple Fruit) Extract | 0.3 |
| Pyridoxine HCl | 0.2 |
| Citric Acid | ad pH 5.0 |
| Taurine | 0.1 |
| Dyes | q.s. |

Example formulation 112: After-Sun Hair and Body Shampoo

| Ingredient | % |
|---|---|
| Aqua | ad 100 |
| Sodium Laureth Sulfate | 7.0 |
| Cocamidopropyl Betaine | 5.0 |
| PEG-18 Glyceryl Oleate/Cocoate | 2.5 |
| Composition A | 2.0 |
| Glycol Distearate | 2.0 |
| Coco Glucoside | 2.0 |
| Laureth-4 | 1.5 |
| Sodium Chloride | 0.9 |
| Panthenol | 0.1 |
| Sodium PCA | 0.7 |
| Polyquaternium-10 | 0.3 |
| Octyldodecyl PCA | 0.2 |
| Glycerin | 0.9 |
| PPG-9 | 0.7 |
| Citric Acid | ad pH 5.5 |
| Tetrasodium EDTA | 0.3 |
| Quaternium-80 | 0.2 |
| Propylene Glycol | 0.3 |
| Isopropyl Alcohol | 0.3 |
| Sodium Benzoate | 0.7 |
| Limonene | 0.1 |
| Linalool | 0.1 |
| Benzyl Salicylate | 0.1 |
| Butylphenyl Methylpropional | 0.1 |
| Ceramide NP | 0.05 |
| Dyes | q.s. |

Example formulation 113: Hair and Body Shampoo & Shower Gel

| Ingredient | % |
|---|---|
| Aqua | ad 100 |
| Sodium Laureth Sulfate | 7.0 |
| Sodium Chloride | 3.8 |
| Cocamidopropyl Betaine | 3.5 |
| Composition A | 3.0 |
| Panthenol | 0.3 |
| Hydrolyzed Keratin | 0.7 |
| Taurine | 0.5 |
| Simmondsia Chinensis Seed Oil | 0.5 |
| Disodium Cocoamphodiacetate | 0.5 |
| Citric Acid | ad pH 5.0 |
| Sodium Benzoate | 0.7 |
| Cocamide MEA | 0.4 |
| PEG-7 Glyceryl Cocoate | 0.3 |
| Polyquaternium-10 | 0.2 |
| Propylene Glycol | 0.3 |
| PEG-40 Hydrogenated Castor Oil | 0.4 |
| Linalool | 0.2 |
| Geraniol | 0.1 |
| Butylphenyl Methylpropional | 0.1 |
| Dyes | q.s. |

### Facial Cleansing Example Formulations

Example formulation 114: Eye Make-Up Remover

| Ingredient | % |
|---|---|
| Aqua | ad 100 |
| Butylene Glycol | 6.0 |
| Propanediol | 2.0 |
| Decyl Glucoside | 1.0 |
| Composition A | 1.0 |
| Glycerin | 1.0 |
| Aloe Barbadensis Leaf Extract | 0.5 |
| Citric Acid | ad pH 7 |
| Bioflavonoids | 0.1 |
| Pantolactone | 0.1 |

Example formulation 115: Eye Make-Up Remover

| Ingredient | % |
|---|---|
| Aqua | ad 100 |
| Cyclopentasiloxane | 8.0 |
| Glycerin | 6.0 |
| Isohexadecane | 6.0 |
| Butylene Glycol | 4.0 |
| Panthenol | 3.0 |
| Composition A | 3.0 |
| Sodium Chloride | 2.0 |
| Disodium EDTA | 1.0 |
| Sodium Hydroxide | 0.2 |
| Citric Acid | ad pH 7 |
| Phenoxyethanol | 0.2 |
| Methylparaben | 0.1 |

Example formulation 116: Micellar Lotion

| Ingredient | % |
|---|---|
| Aqua | ad 100 |
| Poloxamer 124 | 2.0 |
| Composition A | 2.0 |
| Glyceryl Glucoside | 1.5 |
| Glycerin | 1.0 |
| Polyglyceryl-10 Laurate | 1.0 |
| 1,2-Hexandiol | 1.0 |
| Decyl Glucoside | 0.5 |
| Arginine HCl | 0.3 |
| Sodium Cocoamphoacetate | 0.5 |
| Trisodium EDTA | 0.2 |
| Citric Acid | ad pH 6.5 |
| Phenoxyethanol | 0.1 |
| Sodium Hyaluronate | 0.05 |

Example formulation 117: Waterproof Eye Make-Up Removal

| Ingredient | % |
|---|---|
| Aqua | ad 100 |
| Aloe Barbadensis Leaf Juice | 5.0 |
| Sorbitol | 3.0 |
| Glycerin | 3.0 |
| Panthenol | 2.2 |
| Sodium Lauryl Glucose Carboxylate | 2.0 |
| Biosaccharide Gum-1 | 1.5 |
| Lauryl Glucoside | 1.0 |
| Composition A | 0.5 |
| Allantoin | 0.2 |
| Phenoxyethanol | 0.2 |
| Sodium Benzoate | 0.2 |
| Sorbic Acid | q.s. |
| Citric Acid | ad pH 6 |

Example formulation 118: Waterproof Eye Make-Up Removal

| Ingredient | % |
|---|---|
| Aqua | ad 100 |
| Isododecane | 6.0 |
| Isopropyl Palmitate | 4.0 |
| Dimethicone | 3.0 |
| Sodium Ascorbyl Phosphate | 2.0 |
| Sodium Chloride | 2.0 |
| Composition A | 1.5 |
| Caprylyl/Capryl Glucoside | 1.5 |
| Trisodium EDTA | 1.0 |
| Citric Acid | ad pH 7 |
| Sodium Hydroxide | 0.2 |
| Phenoxyethanol | 0.2 |
| Benzethonium Chloride | 0.2 |

Example formulation 119: Micellar Gel

| Ingredient | % |
|---|---|
| Aqua | ad 100 |
| Propanediol | 5.0 |
| Composition A | 3.0 |
| Glycerin, Aqua, Sodium Levulinate, Sodium Anisate | 3.0 |
| Polyglyceryl-4 Caprate | 2.0 |
| Xanthan Gum | 1.5 |
| Lactobacillus Ferment | 0.05 |
| Lactic Acid | ad pH 5.5 |

Example formulation 120: Micellar Gel

| Ingredient | % |
|---|---|
| Aqua | ad 100 |
| Propylene Glycol | 4.0 |
| Glycerin | 4.0 |
| Poloxamer 124 | 2.5 |
| Composition A | 2.5 |
| 1,2-Hexandiol | 2.0 |
| Pentylene Glycol | 1.0 |
| Acrylates/C10-30 Alkyl Acrylate Crosspolymer | 0.5 |
| Sodium Hyaluronate | 0.2 |
| Trisodium EDTA | 0.1 |
| Sodium Hydroxide | ad pH 6 |
| Cetrimonium Chloride | 0.1 |
| Phenoxyethanol | 0.1 |

Example formulation 121: Micellar Make-Up Remover

| Ingredient | % |
|---|---|
| Aqua | ad 100 |
| Aloe Barbadensis Leaf Juice | 4.0 |
| Caprylyl/Capryl Glucoside | 2.0 |
| Glycolipids | 1.5 |
| Composition A | 1.5 |
| Betaine | 1.0 |
| Glycerin | 1.0 |
| Benzyl Alcohol, Caprylyl Glycol, Benzoic Acid | 1.0 |
| Decyl Glucoside | 0.5 |
| Parfum | q. s. |
| Citric Acid | ad pH 7.0 |

Example formulation 122: Facial Cleansing Foam

| Ingredient | % |
|---|---|
| Aqua | ad 100 |
| Glycine Soja Oil | 5.0 |
| Coco-Glucoside | 4.0 |
| Glycerin | 3.5 |
| Composition A | 3.0 |
| Alcohol Denat. | 2.0 |
| Sodium Coco-Sulfate | 1.5 |
| Sodium/Disodium Cocoyl Glutamate | 1.0 |
| Glycolipids | 0.8 |
| Glyceryl Oleate | 0.5 |
| Hamamelis Virginiana Leaf Extract | 0.5 |
| Tocopherol | 0.1 |
| Helianthus Annuus Seed Oil | 0.1 |
| Lactic Acid | ad pH 6.5 |
| Preservative | q.s. |

Example formulation 123: Facial cleansing Foam

| Ingredient | % |
|---|---|
| Aqua | ad 100 |
| Sodium/Disodium Cocoyl Glutamate | 4.0 |
| Composition A | 4.0 |
| Sodium Lauryl Sulfate | 3.0 |
| Parfum | 1.0 |
| Benzyl Alcohol, Caprylyl Glycol, Benzoic Acid | 1.0 |
| Glycerin | 0.8 |
| Sodium Lauryl Glucose Carboxylate | 0.7 |
| Sodium Cottonseedamphoacetate | 0.7 |
| Lauryl Glucoside | 0.5 |
| Citric Acid | ad pH 7.0 |
| Capryloyl Glycine | 0.2 |
| Sodium Hydroxide | 0.2 |
| 1,2-Hexandiol | 0.2 |
| Allantoin | 0.1 |
| Hydroxypropyl Guar Hydroxypropyltrimonium Chloride | 0.1 |
| Tetrasodium EDTA | 0.1 |
| Dehydroacetic Acid | 0.1 |
| Sorbic Acid | 0.1 |
| Tropolone | 0.1 |

Example formulation 124: Cleansing Foam

| Ingredient | % |
|---|---|
| Aqua | ad 100 |
| Coco-Glucoside | 2.0 |
| Glycerin | 1.5 |
| Sodium Coco-Sulfate | 1.5 |
| Glyceryl Oleate | 1.0 |
| Decyl Glucoside | 0.5 |
| Sodium Benzoate | 0.5 |
| Citric Acid | ad pH 5.0 |
| Panthenol | 0.4 |
| Composition A | 0.2 |
| Albumen | 0.1 |
| Potassium Sorbate | 0.1 |
| Tocopherol | 0.1 |
| Hydrogenated Palm Glycerides Citrate | 0.1 |

Example formulation 125: Cleansing Foam

| Ingredient | % |
|---|---|
| Aqua | ad 100 |
| Aloe Barbadensis Leaf Juice | 28.0 |
| Glycerin | 7.0 |
| Decyl Glucoside | 5.0 |
| Ammonium Lauryl Sulfate | 2.0 |
| Benzyl Alcohol, Caprylyl Glocol, Benzoic Acid | 2.0 |
| Parfum | 0.8 |
| Composition A | 0.1 |
| Phytic Acid | 0.1 |
| Sodium Hydroxide | ad pH 7.0 |
| Gluconolactone | 0.1 |

Example formulation 126: Cleansing Foam

| Ingredient | % |
|---|---|
| Aqua | ad 100 |
| Glycerin | 3.0 |
| Decyl Glucoside | 3.0 |
| Glycolipids | 2.0 |
| Pentylene Glycol | 2.0 |
| Composition A | 1.0 |
| Citric Acid | ad pH 6.0 |
| Papain | 0.1 |

Example formulation 127: Pump Foam for Facial Cleansing

| Ingredient | % |
|---|---|
| Aqua | ad 100 |
| Sorbitol | 9.0 |
| Glycerin | 5.0 |
| Decyl Glucoside | 4.0 |
| Disodium Cocoyl Glutamate | 4.0 |
| Glyceryl Glucoside | 2.0 |
| Composition A | 2.0 |
| Propylene Glycol | 2.0 |
| Prunus Amygdalus Dulcis Oil | 2.0 |
| PEG-40 Hydrogenated Castor Oil | 1.5 |
| Sodium Sulfate | 0.5 |
| Citric Acid | ad pH |
| Polyquaternium-10 | 0.5 |
| Sodium Benzoate | 0.2 |
| Aminopeptidase | 0.1 |
| Panthenol | 0.1 |

Example formulation 128: Pump Foam for Facial Cleansing

| Ingredient | % |
|---|---|
| Aqua | ad 100 |
| Sucrose | 12.0 |
| Glycerin | 6.0 |
| Rosa Damascena Flower Water | 5.0 |
| Coco-Glucoside | 4.5 |
| Composition A | 2.5 |
| Aqua, Sodium Levulinate, Potassium Sorbate | 2.0 |
| Sodium Cocoyl Glutamate | 3.0 |
| Polyglyceryl-4 Caprate | 3.0 |
| Betaine | 1.0 |
| Citric Acid | ad pH 5.0 |
| Parfum | 0.8 |
| Sodium Chloride | 0.5 |

Example formulation 129: Micellar Water

| Ingredient | % |
|---|---|
| Aqua | ad 100 |
| PEG-6 Caprylic/capric Glycerides | 3.0 |
| Polysorbate 20 | 2.0 |
| Composition A | 0.8 |
| Butylene Glycol | 0.5 |
| PEG-8 | 0.4 |
| Poloxamer 407 | 0.4 |
| Disodium EDTA | 0.3 |
| Citric Acid | ad pH 6.5 |
| BHT | 0.2 |
| Sodium Benzoate | 0.2 |

Example formulation 130: Refreshing Micellar Water

| Ingredient | % |
|---|---|
| Aqua | ad 100 |
| Methylpropanediol | 3.0 |
| Propylene Glycol | 2.5 |
| Glycerin | 2.5 |
| Sodium Cocoamphoacetate | 2.0 |
| Glycolipids | 1.5 |
| Parfum | 0.8 |
| Composition A | 0.5 |
| Sodium Salicylate | 0.5 |
| Sodium Benzoate | 0.5 |
| Citric Acid | ad pH |
| Sodium Chloride | 0.2 |
| Tetrasodium EDTA | 0.1 |

Example formulation 131: Oil-infused Micellar Water

| Ingredient | % |
|---|---|
| Aqua | ad 100 |
| Isododecane | 7.5 |
| Dimethicone | 7.5 |
| Dicaprylyl Ether | 5.0 |
| Hexylene Glycol | 1.4 |
| Propanediol | 1.0 |
| Composition A | 1.0 |
| Glycolipids | 1.0 |
| Sodium Chloride | 1.0 |
| Trisiloxane | 0.8 |
| Alcohol Denat. | 0.8 |
| Glycerin | 0.8 |
| Propylene Glycol Dicaprylate/Dicaprate | 0.8 |
| Disodium EDTA | 0.5 |
| Hydroxyethylcellulose | 0.5 |
| Phenoxyethanol, Benzoic Acid | 0.5 |
| Decyl Glucoside | 0.5 |
| Caprylyl Glycol | 0.5 |
| Sodium Citrate | 0.3 |
| Citric Acid | ad pH 6 |
| Panthenol | 0.2 |
| Benzophenone-3 | 0.1 |
| Tocopheryl Acetate | 0.1 |
| Tocopherol | 0.1 |
| Zea Mays Oil | 0.1 |
| Chlorphenesin | 0.1 |

Example formulation 132: Facial Tonic

| Ingredient | % |
|---|---|
| Aqua | ad 100 |
| Glycerin. | 2.5 |
| Propanediol | 2.0 |
| Arginine | 1.0 |
| Composition A | 1.0 |
| Salicylic Acid | 1.0 |
| Coco-Betaine | 1.0 |
| Caprylyl/Capryl Glucoside | 0.8 |
| Disodium Cocoyl Glutamate | 0.8 |
| Xanthan Gum | 0.5 |
| Phytic Acid | 0.5 |
| Sodium Chloride | 0.5 |
| Sodium Cocoyl Glutamate | 0.4 |
| Citric Acid | ad pH 5.5 |

Example formulation 133: Facial Tonic

| Ingredient | % |
|---|---|
| Aqua | ad 100 |
| Glycerin | 5.0 |
| Caprylyl/Capryl Glucoside | 2.0 |
| Composition A | 1.5 |
| Glyceryl Caprylate | 1.5 |
| Parfum | 1.0 |
| Sodium Levulinate | 1.0 |
| Sodium Anisate | 1.0 |
| Sodium Salicylate | 1.0 |
| Sodium Cocoyl Glutamate | 0.1 |
| Polyglyceryl-6 Oleate | 0.1 |
| Disodium Cocoyl Glutamate | 0.1 |
| Citric Acid | ad pH 5.5 |
| Sodium Surfactin | 0.05 |

Example formulation 134: Facial Tonic

| Ingredient | % |
|---|---|
| Aqua | ad 100 |
| PEG-6 Caprylic/Capric Glycerides | 4.0 |
| Composition A | 3.0 |
| Fructooligosaccharides | 1.0 |
| Mannitol | 1.0 |
| Xylitol | 1.0 |
| Propylene Glycol | 1.0 |
| Cetrimonium Bromide | 1.0 |
| Disodium EDTA | 0.4 |
| Rhamnose | 0.1 |

Example formulation 135: Micellar Water

| Ingredient | % |
|---|---|
| Aqua | ad 100 |
| Glycerin | 5.0 |
| Polyglyceryl-4 Caprate | 5.0 |
| Betaine | 3.0 |
| Composition A | 2.0 |
| Sodium Levulinate | 2.0 |
| Lactobacillus Ferment | 0.2 |
| Sodium Benzoate | 0.2 |
| Sodium Hydroxide | 0.2 |
| Parfum | 0.1 |
| Lactic Acid | ad pH 5.8 |

Example formulation 136: Micellar Water

| Ingredient | % |
|---|---|
| Aqua | ad 100 |
| Glycerin | 6.0 |
| Poloxamer 184 | 2.5 |
| Phenoxyethanol | 1.0 |
| PEG-40 Hydrogenated Castor Oil | 1.0 |
| Lactobacillus Extract, Propylene Glycol | 0.8 |
| Composition A | 0.5 |
| Parfum | 0.5 |
| Citric Acid | ad pH 6.4 |
| Sodium Hydroxide | 0.2 |
| Disodium EDTA | 0.1 |

Example formulation 137: Micellar Water

| Ingredient | % |
|---|---|
| Aqua | ad 100 |
| Composition A | 5.0 |
| PEG-40 Hydrogenated Castor Oil | 2.5 |
| Glycerin | 2.5 |
| Prunus Amygdalus Dulcis Oil | 1.0 |
| Sorbitol | 1.0 |
| Decyl Glucoside | 1.0 |
| 1,2-Hexandiol | 0.1 |
| Glyceryl Glucoside | 0.5 |
| Poloxamer 124 | 0.5 |
| Propylene Glycol | 0.5 |
| Disodium Cocoyl Glutamate | 0.5 |
| Sodium Chloride | 0.2 |
| Trisodium EDTA | 0.2 |
| Polyquaternium-10 | 0.2 |
| Citric Acid | ad pH 6.2 |
| Sodium Acetate | 0.1 |
| Phenoxyethanol | 0.1 |

Example formulation 138: Micellar Water

| Ingredient | % |
|---|---|
| Aqua | ad 100 |
| Composition A | 5.0 |
| Glycerin | 4.0 |
| Coco-Glucoside | 2.0 |
| Polysorbate 80 | 0.2 |
| Calendula Officinalis Flower Extract | 2.0 |
| Caprylyl Glycol | 1.5 |
| Citric Acid | ad pH |
| Sodium Benzoate | 0.9 |
| Parfum | 0.6 |
| Isoleucine | 0.2 |
| Panthenol | 0.2 |
| PPG-1-PEG-9 Lauryl Glycol Ether | 0.2 |
| Propylene Glycol | 0.2 |
| Phytosphingosine | 0.1 |
| Sodium Chloride | 0.2 |

Example formulation 139: Micellar Water

| Ingredient | % |
|---|---|
| Aqua | ad 100 |
| Hexylene Glycol | 1.4 |
| Glycerin | 1.2 |
| Composition A | 1.0 |
| Poloxamer 184 | 1.0 |
| Disodium Cocoamphodiacetate | 1.0 |
| Disodium EDTA | 0.5 |
| Polyaminopropyl Biguanide | 0.2 |
| Lactic Acid | ad pH 5.4 |

Example formulation 140: Micellar Water

| Ingredient | % |
|---|---|
| Aqua | ad 100 |
| Glycerin | 5.0 |
| Alcohol Denat. | 2.0 |
| Sodium Lactate | 2.0 |
| Sodium PCA | 1.0 |
| Composition A | 0.5 |
| Parfum | 0.3 |
| Aloe Barbadensis Leaf Juice | 0.3 |
| Cocoyl Glutamic Acid | 0.2 |
| Sodium Cocoyl Glutamate | 0.2 |
| Glycolipids | 0.1 |
| Citric Acid | 0.1 |
| Lactic Acid | 0.1 |
| Sodium Hyaluronate | 0.1 |
| Phytosphingosine HCl | 0.1 |
| Benzyl Alcohol | 0.1 |

Example formulation 141: Face Cleanser and Scrub

| Ingredient | % |
|---|---|
| Aqua | ad 100 |
| Sodium Coco-Sulfate | 4.0 |
| Composition A | 2.0 |
| Glycerin | 2.0 |
| Sodium Cocoyl Glycinate | 1.5 |
| Silica | 1.5 |
| Acrylates Copolymer | 0.8 |
| PEG-7 Glyceryl Cocoate | 0.7 |
| Sodium Chloride | 0.5 |
| Polysorbate 20 | 0.5 |
| Cocamidopropyl Betaine | 0.5 |
| Panthenol | 0.2 |
| Parfum | 0.2 |
| Charcoal Powder | 0.2 |
| Salix Alba (Willow) Bark Extract | 0.2 |
| Glycol Distearate | 0.1 |
| Lecithin | 0.1 |
| Laureth-4 | 0.1 |
| Citric Acid | ad pH 5.6 |
| Lactic Acid | 0.1 |
| Tocopherol | 0.1 |
| Ascorbyl Palmitate | 0.1 |
| Disodium EDTA | 0.1 |
| Phenoxyethanol | 0.1 |
| Ethylhexylglycerin | 0.1 |
| Sodium Benzoate | 0.1 |
| Benzyl Alcohol | 0.1 |
| Potassium Sorbate | 0.1 |

### Microemulsion Example Formulations

Example formulation 142: Microemulsion for Make-Up Removal

| Ingredient | % |
|---|---|
| Water | ad 100 |
| 1,3-Butanediol | 15.0 |
| Polyglyceryl-4 Caprate | 6.0 |
| Polyglyceryl-3 Caprate | 6.0 |
| Composition A | 6.0 |
| Glycerin | 6.0 |
| Isohexadecane | 3.0 |
| Sodium Benzoate | 0.8 |
| Citric Acid | ad pH 5.0 |

Example formulation 143: Microemulsion for Make-Up Removal

| Ingredient | % |
|---|---|
| Water | ad 100 |
| 1,3-Butanediol | 7.5 |
| Polyglyceryl-4 Caprate | 5.5 |
| Glycolipids | 3.0 |
| Composition A | 1.5 |
| Glycerin | 5.0 |
| Sodium Benzoate | 0.8 |
| Citric Acid | Ad pH 5.0 |

### Toothpaste Example Formulations

Example formulation 144: Toothpaste

| Ingredient | % |
|---|---|
| Aqua | ad 100 |
| Glycerin | 29.0 |
| Sodium Metaphosphate | 12.0 |
| Hydrated Silica (Hydrated) | 10.0 |
| Xylitol | 10.0 |
| Composition A | 1.0 |
| Olaflur | 0.13 |
| Cocamidopropyl Betaine | 0.5 |
| Propylene Glycol | 1.0 |
| Chamomilla Recutita Flower Extract | 0.4 |
| Alcohol Denat | 0.5 |
| Panthenol | 0.1 |
| Sodium Fluoride | 0.1 |
| Hydroxyethylcellulose | 1.0 |
| Titanium Dioxide | 0.4 |
| Aroma | 1.0 |
| Citronellol | 0.0 |
| Eucalyptol | 0.2 |
| Eugenol | 0.1 |
| Menthol | 0.15 |
| Dyes, pH adjuster, preservative | q.s. |

Example formulation 145: Toothpaste

| Ingredient | % |
|---|---|
| Aqua | ad 100 |
| Hydrated Silica (Hydrated) | 22.0 |
| Sorbitol | 30.0 |
| Hydroxyethylcellulose | 1.5 |
| Composition A | 1.0 |
| Olaflur | 0.19 |
| Aroma | 1.0 |
| Limonene | 0.1 |
| Titanium Dioxide | 0.3 |
| Saccharin | 0.2 |
| Dyes, pH adjuster, preservative | q.s. |

Example formulation 146: Toothpaste

| Ingredient | % |
|---|---|
| Sorbitol | 45.0 |
| Aqua | ad 100 |
| Hydrated Silica (Hydrated) | 18.0 |
| Sodium Lauryl Sulfate | 1.05 |
| Composition A | 0.75 |
| PEG-32 | 1.0 |
| Flavor | 1.0 |
| Cellulose Gum | 0.8 |
| Cocamidopropyl Betaine | 0.35 |
| Sodium Saccharin | 0.2 |
| Sodium Fluoride | 0.23 |
| Zinc Sulfate | 0.3 |
| Mica | 0.5 |
| Dyes solution (1% FD&C Blue No. 1) | 0.02 |
| Titanium Dioxide | 0.3 |
| Eugenol | 0.2 |
| pH adjuster, preservative | q.s. |

Example formulation 147: Toothpaste

| Ingredient | % |
|---|---|
| Glycerin | 35.0 |
| Hydrated Silica (Hydrated) | 22.0 |
| Aqua | ad 100 |
| Sodium Bicarbonate | 20.0 |
| PEG-12 | 3.0 |
| Composition A | 1.5 |
| Sodium Lauryl Sulfate | 1.0 |
| Flavor | 1.0 |
| Sodium Hydroxide | 0.9 |
| Cellulose Gum | 0.9 |
| Chondrus Crispus | 0.5 |
| Sodium Saccharin | 0.1 |
| Calcium Peroxide | 0.1 |
| Titanium Dioxide | 0.4 |
| Sodium monofluorophosphate | 0.76 |
| PH adjuster, preservative | q.s. |

Example formulation 148: Toothpaste

| Ingredient | % |
|---|---|
| Aqua | ad 100 |
| Glycerin | 33.0 |
| Hydrated Silica (Hydrated) | 24.0 |
| Composition A | 1.5 |
| Cellulose Gum | 0.8 |
| Aroma | 0.9 |
| Sodium Fluoride | 0.23 |
| Sodium Saccharin | 0.2 |
| Commiphora Myrrha Oil | 0.05 |
| Salvia Officinalis Oil | 0.05 |
| Mentha Piperita Oil | 0.2 |
| Chamomilla Recutita Flower Extract | 0.2 |
| Limonene | 0.1 |
| Titanium Dioxide | 0.3 |
| Dyes solution (1% FD&C Blue No. 1) | 0.05 |
| pH adjuster, preservative | q.s. |

Example formulation 149: Toothpaste

| Ingredient | % |
|---|---|
| Glycerin | 27.0 |
| Hydrated Silica (Hydrated) | 21.0 |
| Sodium Hexametaphosphate | 13.0 |
| Aqua | ad 100 |
| PEG-6 | 5.0 |
| Flavor | 1.0 |
| Trisodium Phosphate | 1.0 |
| Sodium Lauryl Sulfate | 1.05 |
| Composition A | 0.35 |
| Chondrus Crispus | 0.7 |
| Cocamidopropyl Betaine | 0.1 |
| Sodium Saccharin | 0.2 |
| Polyethylene Glycol | 0.5 |
| Xanthan Gum | 0.7 |
| Sucralose | 0.5 |
| Mica | 0.3 |
| Titanium Dioxide | 0.5 |
| Sodium Fluoride | 0.32 |
| Dyes, pH adjuster, preservative | q.s. |

Example formulation 150: Toothpaste

| Ingredient | % |
|---|---|
| Aqua | ad 100 |
| Sorbitol | 29.0 |
| Hydrated Silica (Hydrated) | 22.0 |
| Disodium Pyrophosphate | 5.0 |
| Composition A | 1.0 |
| Glycolipids | 1.0 |
| Cellulose Gum | 1.0 |
| Aroma | 0.9 |
| Sodium Bicarbonate | 0.8 |
| Carbomer | 0.8 |
| Sodium Fluoride | 0.32 |
| Sodium Saccharin | 0.1 |
| Xanthan Gum | 0.5 |
| Titanium Dioxide | 0.5 |
| Limonene | 0.2 |
| Dyes, pH adjuster, preservative | q.s. |

Example formulation 151: Toothpaste

| Ingredient | % |
|---|---|
| Aqua | ad 100 |
| Hydrated Silica (Hydrated) | 21.0 |
| Sorbitol | 19.0 |
| Glycerin | 15.0 |
| Propylene Glycol | 5.0 |
| Tetrapotassium Pyrophosphate | 5.0 |
| Pentasodium Triphosphate | 5.0 |
| Sodium C14-16 Olefin Sulfonate | 0.5 |
| Glycolipids | 0.5 |
| Composition A | 0.5 |
| Disodium Pyrophosphate | 0.7 |
| Aroma | 0.3 |
| Titanium Dioxide | 0.4 |
| Xanthan Gum | 0.7 |
| Sodium Fluoride | 0.32 |
| Sodium Saccharin | 0.15 |
| Allantoin | 0.2 |
| Chamomilla Recutita Flower Extract | 0.1 |
| Salvia Officinalis Leaf Extract | 0.2 |
| Zinc Chloride | 0.3 |
| Dyes solution (1% FD&C Blue No. 1) | 0.05 |
| pH adjuster, preservative | q.s. |

Example formulation 152: Toothpaste

| Ingredient | % |
|---|---|
| Aqua | ad 100 |
| Hydrated Silica (Hydrated) | 21.0 |
| Xylitol | 10.0 |
| Sorbitol | 10.0 |
| Composition A | 1.5 |
| Betaine | 0.5 |
| Aloe Barbadensis Leaf Juice Powder | 0.3 |
| Xanthan Gum | 0.5 |
| Zinc Gluconate | 0.5 |
| Sodium Coco-sulfate | 0.5 |
| Mentha Spicata Crispa Herb Oil | 0.1 |
| Calendula Officinalis Flower Extract | 0.2 |
| Chamomilla Recutita Flower Extract | 0.2 |
| L-limonene | 0.1 |
| Mentha Arvensis Leaf Oil | 0.1 |
| Sodium Fluoride | 0.32 |
| Titanium Dioxide | 0.4 |
| PH adjuster, preservative | q.s. |

Example formulation 153: Toothpaste

| Ingredient | % |
|---|---|
| Aqua | ad 100 |
| Hydroxyapatite | 20.0 |
| Propylene Glycol | 10.0 |
| Glycerin | 10.0 |
| Sorbitol | 10.0 |
| Tetrapotassium Pyrophosphate | 7.0 |
| Silica | 7.0 |
| Composition A | 1.0 |
| Aroma | 1.0 |
| Cellulose Gum | 1.0 |
| Sodium C14-16 Olefin Sulfonate | 1.0 |
| Sodium Fluoride | 0.32 |
| Sodium Cocoyl Isethionate | 0.5 |
| Sodium Saccharin | 0.15 |
| Limonene | 0.2 |
| Titanium Dioxide | 0.4 |
| Dyes, pH adjuster, preservative | q.s. |

Example formulation 154: Toothpaste

| Ingredient | % |
|---|---|
| Aqua | ad 100 |
| Hydrogenated Starch Hydrolysate (Hydrogenated, Hydrolysed) | 20.0 |
| Hydrated Silica (Hydrated), | 15.0 |
| Glycerin | 15.0 |
| Alumina | 10.0 |
| Sodium Lauryl Sulfate | 1.05 |
| PEG-12 | 1.0 |
| Composition A | 0.75 |
| Perlite | 0.5 |
| Pentapotassium Triphosphate | 0.8 |
| Pentasodium Triphosphate | 0.8 |
| Sodium Monofluorophosphate | 0.76 |
| Cellulose Gum | 0.9 |
| Sodium Saccharin | 0.2 |
| Cocamidopropyl Betaine | 0.35 |
| Aroma | 1.0 |
| Limonene | 0.1 |
| Dyes solution (1% FD&C Blue No. 1) | 0.05 |
| Titanium Dioxide | 0.3 |
| pH adjuster, preservative | q.s. |

Example formulation 155: Toothpaste

| Ingredient | % |
|---|---|
| Dicalcium Phosphate Dihydrate (Dihydrate) | 25.0 |
| Aqua | ad 100 |
| Sorbitol | 20.0 |
| Glycerin | 20.0 |
| Methyl Salicylate | 1.0 |
| Sodium Lauryl Sulfate | 0.9 |
| Composition A | 0.9 |
| Titanium Dioxide | 0.5 |
| Chondrus Crispus | 0.8 |
| Cellulose Gum | 0.5 |
| Sodium Silicate | 0.3 |
| Hydrated Silica (Hydrated) | 1.0 |
| Thymol | 0.1 |
| Sodium Saccharin | 0.2 |
| Sodium Hydroxide | 0.5 |
| Menthol | 0.3 |
| Dyes solution (1% FD&C Blue No. 1) | 0.02 |
| pH adjuster, preservative | q.s. |

Example formulation 156: Toothpaste

| Ingredient | % |
|---|---|
| Aqua | ad 100 |
| Hydrated Silica (Hydrated) | 21.0 |
| Sorbitol | 21.0 |
| Glycerin | 18.0 |
| PEG-6 | 5.0 |
| Composition A | 1.5 |
| Xanthan Gum | 0.8 |
| Flavor | 0.7 |
| Titanium Dioxide | 0.5 |
| Sodium Citrate | 0.4 |
| Sodium Fluoride | 0.32 |
| Zinc Chloride | 0.5 |
| Sodium Saccharin | 0.2 |
| Chondrus Crispus | 0.7 |
| Limonene | 0.1 |
| Dyes solution (1% FD&C Blue No. 1) | 0.04 |
| pH adjuster, preservative | q.s. |

Example formulation 157: Toothpaste

| Ingredient | % |
|---|---|
| Aqua | ad 100 |
| Sorbitol | 36.0 |
| Hydrated Silica (Hydrated), | 18.0 |
| Glycerin | 18.0 |
| Potassium Nitrate | 1.0 |
| Aroma | 1.0 |
| Cocamidopropyl Betaine | 1.0 |
| Composition A | 1.0 |
| Xanthan Gum | 0.7 |
| Sodium Saccharin | 0.2 |
| Sodium Fluoride | 0.32 |
| Mica | 0.4 |
| Titanium Dioxide | 0.4 |
| Sodium Hydroxide | 0.5 |
| Limonene | 0.1 |
| Eugenol | 0.1 |
| Dyes, pH adjuster, preservative | q.s. |

Example formulation 158: Toothpaste

| Ingredient | % |
|---|---|
| Glycerin | 33.0 |
| PEG-8 | 5.0 |
| Hydrated Silica (Hydrated), | 5.0 |
| Calcium Sodium Phosphosilicate | 3.0 |
| Cocamidopropyl Betaine | 1.0 |
| Composition A | 1.0 |
| Sodium Methyl Cocoyl Taurate | 0.3 |
| Aroma | 0.8 |
| Titanium Dioxide | 0.5 |
| Carbomer | 0.5 |
| Sodium Saccharin | 0.2 |
| Sodium Fluoride | 0.32 |
| Limonene | 0.2 |
| Dyes, pH adjuster, preservative | q.s. |

Example formulation 159: Toothpaste

| Ingredient | % |
|---|---|
| Aqua | ad 100 |
| Sorbitol | 63.0 |
| Hydrated Silica (Hydrated) | 14.0 |
| Silica | 7.0 |
| Composition As | 1.0 |
| Titanium Dioxide | 0.6 |
| Maris Sal | 0.5 |
| Glycolipdis | 0.5 |
| Sodium Cocoyl Glutamate | 0.5 |
| Disodium Cocoyl Glutamate | 0.5 |
| Xanthan Gum | 0.7 |
| Sodium Fluoride | 0.32 |
| Echinacea Purpurea Extract (Extract) | 0.1 |
| Arnica Montana Flower Extract | 0.1 |
| Mentha Piperita Leaf Water | 0.1 |
| Myrtus Communis Leaf Water | 0.1 |
| Glycerin | 1.0 |
| Menthol | 0.2 |
| Aroma | 1.0 |
| L-limonene | 0.2 |
| Dyes, pH adjuster, preservative | q.s. |

Example formulation 160: Toothpaste

| Ingredient | % |
|---|---|
| Sorbitol | 33.0 |
| Aqua | ad 100 |
| Hydrated Silica (Hydrated) | 18.0 |
| Glycerin | 15.0 |
| PEG-32 | 4.0 |
| Sodium Lauryl Sulfate | 1.5 |
| Cellulose Gum | 1.0 |
| Composition A | 1.0 |
| Sodium Saccharin | 0.3 |
| Eucalyptol | 0.2 |
| Methyl Salicylate | 0.5 |
| Thymol | 0.1 |
| Phosphoric Acid | 0.3 |
| Menthol | 0.4 |
| Zinc Citrate | 0.5 |
| Sodium Phosphate | 0.2 |
| Xanthan Gum | 0.5 |
| Benzoic Acid | 0.2 |
| Sodium Monofluorophosphate | 0.76 |
| Flavor | 1.0 |
| Dyes solution (1% FD&C Blue No. 1) | 0.05 |
| pH adjuster, preservative | q.s. |

Example formulation 161: Toothpaste

| Ingredient | % |
|---|---|
| Hydrogenated Starch Hydrolysate (Hydrogenated, Hydrolysed) | 30.0 |
| Aqua | ad 100 |
| Hydrated Silica (Hydrated) | 22.0 |
| PEG-32 | 4.0 |
| Composition A | 1.0 |
| Aroma | 1.0 |
| Cellulose Gum | 1.0 |
| Sodium Fluoride | 0.32 |
| Sodium Saccharin | 0.25 |
| PVM/MA Copolymer | 0.7 |
| Trisodium Phosphate | 0.8 |
| Calcium Aluminum Borosilicate | 0.4 |
| Glycerin | 0.9 |
| Lecithin | 0.2 |
| Tin Oxide | 0.2 |
| Limonene | 0.1 |
| Dyes solution (1% FD&C Blue No. 1) | 0.04 |
| Titanium Dioxide | 0.3 |
| pH adjuster, preservative | q.s. |

Example formulation 162: Toothpaste

| Ingredient | % |
|---|---|
| Aqua | ad 100 |
| Sorbitol | 50.0 |
| Hydrated Silica (Hydrated), | 14.0 |
| Silica Dimethyl Silylate | 3.5 |
| Hydroxyethylcellulose | 3.5 |
| Composition A | 1.05 |
| PEG-40 Hydrogenated Castor Oil (Hydrogenated) | 0.8 |
| Aroma | 0.9 |
| Sodium Gluconate | 0.4 |
| Limonene | 0.1 |
| PEG-3 tallow aminopropyl amine | 0.5 |
| Olaflur | 0.06 |
| Stannous Fluoride | 0.4 |
| Sodium Saccharin | 0.2 |
| Potassium Hydroxide | 0.4 |
| Hydrochloric Acid | 0.2 |
| Dyes solution (1% FD&C Blue No. 1) | 0.04 |
| pH adjuster, preservative | q.s. |

Example formulation 163: Toothpaste

| Ingredient | % |
|---|---|
| Aqua | ad 100 |
| Sorbitol | 45.0 |
| Hydrated Silica (Hydrated) | 21.0 |
| Sodium Lauryl Sulfate | 1.5 |
| Composition A | xx |
| Cellulose Gum | 1.0 |
| Aroma | 1.0 |
| Zinc Citrate | 0.5 |
| Chondrus Crispus Powder (Powdered) | 0.8 |
| Sodium Saccharin | 0.2 |
| Sodium Fluoride | 0.1 |
| Titanium Dioxide | 0.5 |
| Hydroxyethylcellulose | 0.3 |
| Sodium Citrate | 0.2 |
| Stannous Fluoride | 0.4 |
| Dyes, pH adjuster, preservative | q.s. |

Example formulation 164: Toothpaste

| Ingredient | % |
|---|---|
| Sodium Bicarbonate | 35.00 |
| PEG-8 | 5.0 |
| Hydrated Silica (Hydrated | 18.0 |
| Glycerin | 18.0 |
| Composition A | 1.0 |
| Sodium Lauryl Sulfate | 0.5 |
| Sodium Saccharin | 0.5 |
| Aroma | 0.9 |
| Dipotassium Phosphate | 0.8 |
| Sodium Carbonate | 0.5 |
| Sodium Fluoride | 0.32 |
| PEG/PPG-116/66 Copolymer | 0.8 |
| Calcium Peroxide | 0.5 |
| Calcium Hydroxide | 0.4 |
| Dyes solution (1% FD&C Blue No. 1) | 0.04 |
| Sodium Silicate | 0.5 |
| Limonene | 0.1 |
| Silica | 0.9 |
| Titanium Dioxide | 0.3 |
| Dyes, pH adjuster, preservative | q.s. |

Example formulation 165: Toothpaste

| Ingredient | % |
|---|---|
| Glycerin | 33.0 |
| Hydrated Silica | 22.0 |
| Aqua | ad 100 |
| Sorbitol | 15.0 |
| Tetrapotassium Pyrophosphate | 3.5 |
| Composition A | 1.5 |
| Titanium Dioxide | 0.4 |
| Sodium Lauroyl Sarcosinate | 0.5 |
| Cellulose Gum | 0.8 |
| Lauryl Glucoside | 0.5 |
| PVP | 0.5 |
| Cocamidopropyl Betaine | 0.5 |
| Xanthan Gum | 0.5 |
| Stannous Fluoride | 0.4 |
| Dyes, pH adjuster, preservative | q.s. |

Example formulation 166: Toothpaste

| Ingredient | % |
|---|---|
| Aqua | ad 100 |
| Hydrated Silica (Hydrated) | 20.0 |
| Sorbitol | 18.0 |
| Xylitol | 14.0 |
| Composition A | 1.2 |
| Propylene Glycol | 1.0 |
| Glycerin | 1.0 |
| Sodium C14-16 Olefin Sulfonate | 0.3 |
| Aroma | 1.0 |
| Xanthan Gum | 0.5 |
| Disodium Phosphate | 0.4 |
| Sodium Fluoride | 0.32 |
| Zinc Chloride | 0.5 |
| Sodium Saccharin | 0.2 |
| Citric Acid | 0.2 |
| Titanium Dioxide | 0.4 |
| Dyes, pH adjuster, preservative | q.s. |

Example formulation 167: Toothpaste

| Ingredient | % |
|---|---|
| Aqua | ad 100 |
| Hydrogenated Starch Hydrolysate (Hydrogenated, Hydrolysed) | 25.0 |
| Hydrated Silica (Hydrated) | 21.0 |
| Hydroxyapatite | 4.5 |
| PEG-32 | 4.5 |
| Composition A | 1.0 |
| Sodium Lauryl Sulfate | 0.5 |
| Aroma | 0.9 |
| Sodium Monofluorophosphate | 0.76 |
| Trisodium Phosphate | 0.5 |
| Cellulose Gum | 1.0 |
| Sodium Saccharin | 0.2 |
| Sodium Hydroxide | 0.3 |
| Limonene | 0.1 |
| Dyes solution (1% FD&C Blue No. 1) | 0.05 |
| Titanium Dioxide | 0.4 |
| pH adjuster, preservative | q.s. |

Example formulation 168: Toothpaste

| Ingredient | % |
|---|---|
| Glycerin | 32.0 |
| Hydrated Silica (Hydrated) | 21.0 |
| Aqua | ad 100 |
| Sodium Bicarbonate | 30.0 |
| PEG-12 | 3.0 |
| Composition A | 1.5 |
| Sodium Lauryl Sulfate | 1.0 |
| Flavor | 1.0 |
| Sodium Hydroxide | 1.0 |
| Cellulose Gum | 1.0 |
| Chondrus Crispus | 0.7 |
| Sodium Saccharin | 0.15 |
| Calcium Peroxide | 0.5 |
| Titanium Dioxide | 0.5 |
| Sodium Monofluorophosphate | 0.76 |
| Dyes, pH adjuster, preservative | q.s. |

Example formulation 169: Toothpaste

| Ingredient | % |
|---|---|
| Aqua | ad 100 |
| Sorbitol | 63.0 |
| Hydrated Silica (Hydrated) | 22.0 |
| Propylene Glycol | 1.5 |
| Cellulose Gum | 1.5 |
| Composition A | 1.0 |
| Sodium C 14-16 Olefin Sulfonate | 0.5 |
| Aroma | 0.8 |
| Calcium Glycerophosphate | 0.4 |
| Sodium Fluoride | 0.11 |
| Sodium Saccharin | 0.4 |
| Cocamidopropyl Betaine | 0.5 |
| Citral | 0.1 |
| Geraniol | 0.1 |
| Limonene | 0.1 |
| Linalool | 0.1 |
| Titanium Dioxide | 0.2 |
| Dyes, pH adjuster, preservative | q.s. |

Example formulation 170: Toothpaste

| Ingredient | % |
|---|---|
| Aqua | ad 100 |
| Calcium Carbonate | 30.0 |
| Silica | 7.0 |
| Glycerin | 5.0 |
| Hydrated Silica | 2.5 |
| Composition A | 1.5 |
| Aroma | 1.0 |
| Chondrus Crispus Powder | 0.5 |
| Cellulose Gum | 0.5 |
| Chlorhexidine Gluconate | 0.1 |
| Limonene | 0.15 |
| Sodium Saccharin | 0.15 |
| Titanium Dioxide | 0.1 |
| Sodium Fluoride | 0.32 |
| Dyes, pH adjuster, preservative | q.s. |

Example formulation 171: Toothpaste

| Ingredient | % |
|---|---|
| Aqua | ad 100 |
| Hydrogenated Starch Hydrolysate | 20.0 |
| Hydrated Silica | 15.0 |
| PEG-32 | 4.0 |
| Glycolipids | 1.5 |
| Composition A | 1.0 |
| Propylene Glycol | 0.9 |
| Aroma | 0.9 |
| Sodium Lauryl Sulfat | 0.5 |
| Hydroxyethylcellulose | 0.5 |
| Olaflur | 0.13 |
| Titanium Dioxide | 0.1 |
| Sodium Fluoride | 0.1 |
| Sodium Saccharin | 0.2 |
| Chamomilla Recutita Flower Extract | 0.2 |
| Chlorhexidine Digluconate | 0.1 |
| Limonene | 0.1 |
| Citral | 0.1 |
| Dyes, pH adjuster, preservative | q.s. |

Example formulation 172: Toothpaste

| Ingredient | % |
|---|---|
| Aqua | ad 100 |
| Sorbitol | 20.0 |
| Hydrated Silica | 15.0 |
| Silica | 2.0 |
| Composition A | 1.0 |
| Cocamidopropyl Betaine | 0.75 |
| Titanium Dioxide | 0.15 |
| Flavor | 0.8 |
| Linalool | 0.2 |
| Sodium Monofluorophosphate | 0.76 |
| Sodium Fluoride | 0.1 |
| Sodium Saccharin | 0.25 |
| Cellulose Gum | 0.5 |
| Limonene | 0.1 |
| Chlorhexidine Digluconate | 0.12 |
| Dyes, pH adjuster, preservative | q.s. |

Example formulation 173: Toothpaste

| Ingredient | % |
|---|---|
| Aqua | ad 100 |
| Hydrated Silica | 15.0 |
| Glycerin | 15.0 |
| Potassium Nitrate | 5.0 |
| Sorbitol | 4.5 |
| Sodium Cocoamphoacetate | 0.5 |
| Composition A | 0.5 |
| PEG-40 Hydrogenated Castor Oil | 0.5 |
| Carboxymethyl Cellulose | 0.5 |
| Titanium Dioxide | 0.1 |
| Sodium Fluoride | 0.32 |
| Sodium Saccharin | 0.2 |
| Aroma | 0.2 |
| Chlorhexidine Digluconate | 0.12 |
| Dyes, pH adjuster, preservative | q.s. |

Example formulation 174: Toothpaste for Kids

| Ingredient | % |
|---|---|
| Aqua | ad 100 |
| Sorbitol | 28.0 |
| Hydrated Silica (Hydrated) | 18.0 |
| Hydroxyethylcellulose | 1.5 |
| Titanium Dioxide | 0.5 |
| Cocamidopropyl Betaine | 0.5 |
| Composition A | 0.5 |
| Olaflur | 0.07 |
| Aroma | 0.8 |
| Limonene | 0.1 |
| Sodium Saccharin | 0.2 |
| Hydrochloric Acid | 0.7 |
| Dyes, pH adjuster, preservative | q.s. |

Example formulation 175: Toothpaste for Children

| Ingredient | % |
|---|---|
| Sorbitol | 33.0 |
| Aqua | ad 100 |
| Hydrated Silica (Hydrated) | 22.0 |
| Composition A | 1.0 |
| Sodium C14-16 Olefin Sulfonate | 1.0 |
| Aroma | 0.8 |
| Cellulose Gum | 0.8 |
| Calcium Glycerophosphate | 0.5 |
| Olaflur | 0.07 |
| Sodium Fluoride | 0.1 |
| Propylene Glycol | 0.5 |
| Sodium Saccharin | 0.2 |
| Cocamidopropyl Betaine | 0.35 |
| Mica | 0.5 |
| Citral | 0.1 |
| Limonene | 0.1 |
| Linalool | 0.1 |
| Titanium Dioxide | 0.4 |
| Dyes solution (1% FD&C Blue No. 1) | 0.05 |
| Olaflur | 0.07 |
| pH adjuster, preservative | q.s. |

Example formulation 176: Baby Toothpaste

| Ingredient | % |
|---|---|
| Aqua | ad 100 |
| Hydrogenated Starch Hydrolysate (Hydrogenated, Hydrolysed) | 14.0 |
| Hydrated Silica (Hydrated) | 14.0 |
| PEG-32 | 3.5 |
| Silica | 7.0 |
| Composition A | 1.5 |
| Cellulose Gum | 1.0 |
| Aroma | 1.0 |
| Propylene Glycol | 0.7 |
| PEG-40 Hydrogenated Castor Oil (Hydrogenated), | 0.8 |
| Olaflur | 0.06 |
| Sodium Chloride | 0.2 |
| Sodium Saccharin | 0.15 |
| Tocopheryl Acetate | 0.1 |
| Glycerin | 1.0 |
| Retinyl Palmitate | 0.1 |
| Dyes solution (1% FD&C Blue No. 1) | 0.02 |
| pH adjuster, preservative | q.s. |

Example formulation 177: Toothpaste for Children

| Ingredient | % |
|---|---|
| Aqua | ad 100 |
| Hydrated Silica (Hydrated) | 22.0 |
| Glycerin | 20.0 |
| Xylitol | 10.0 |
| Composition A | 1.5 |
| Propylene Glycol | 0.7 |
| Xanthan Gum | 0.5 |
| Titanium Dioxide | 0.5 |
| Flavor | 0.9 |
| Sodium Lauroyl Sarcosinate | 0.5 |
| Disodium EDTA | 0.3 |
| Sodium Monofluorophosphate | 0.76 |
| Sodium Chloride | 0.2 |
| Dyes, pH adjuster, preservative | q.s. |

Example formulation 178: Kids Toothpaste

| Ingredient | % |
|---|---|
| Aqua | ad 100 |
| Sorbitol | 40.0 |
| Hydrated Silica (Hydrated) | 17.0 |
| Glycerin | 26.0 |
| Composition A | 1.5 |
| Xanthan Gum | 1.0 |
| Steareth-30 | 1.0 |
| Chondrus Crispus | 1.0 |
| Aroma | 1.0 |
| Disodium Phosphate | 0.5 |
| Sodium Benzoate | 0.2 |
| Amyloglucosidase | 0.1 |
| Citric Acid | 0.3 |
| Zinc Gluconate | 0.5 |
| Glucose Oxidase | 0.1 |
| Sodium Fluoride | 0.22 |
| Sodium Saccharin | 0.15 |
| Potassium Thiocyanate | 0.1 |
| Lactoperoxidase | 0.1 |
| Titanium Dioxide | 0.3 |
| Dyes, pH adjuster, preservative | q.s. |

Example formulation 179: 2 in 1 Toothpaste + Mouthwash

| Ingredient | % |
|---|---|
| Sorbitol | 38.0 |
| Aqua | ad 100 |
| Hydrated Silica (Hydrated) | 21.0 |
| Pentasodium Triphosphate | 5.0 |
| Sodium Lauryl Sulfate | 0.35 |
| Composition A | 1.05 |
| PEG-32 | 1.0 |
| Aroma | 1.0 |
| Alumina | 0.5 |
| Alcohols | 0.7 |
| Xanthan Gum | 0.8 |
| Sodium Fluoride | 0.23 |
| Sodium Saccharin | 0.2 |
| Disodium Phosphate | 0.5 |
| Cocamidopropyl Betaine | 0.35 |
| Zinc Sulfate | 0.5 |
| Trisodium Phosphate | 0.3 |
| Sodium Chloride | 0.4 |
| Sodium Sulfate | 0.2 |
| Limonene | 0.1 |
| Titanium Dioxide | 0.5 |
| Dyes, pH adjuster, preservative | q.s. |

Example formulation 180: 2 in 1 Toothpaste + Mouthwash

| Ingredient | % |
|---|---|
| Sorbitol | 29.0 |
| Aqua | ad 100 |
| Hydrated Silica | 15.0 |
| PEG-32 | 3.0 |
| Sodium Lauryl Sulfate | 0.5 |
| Composition A | 0.5 |
| Glycolipids | 0.5 |
| Aroma | 0.9 |
| Alcohols | 0.7 |
| Xanthan Gum | 0.5 |
| PEG-30 Glyceryl Stearate | 0.5 |
| Sodium Fluoride | 0.05 |
| Sodium Saccharin | 0.7 |
| Disodium Phosphate | 0.3 |
| Cocamidopropyl Betaine | 0.5 |
| Zinc Sulfate | 0.3 |
| Trisodium Phosphate | 0.5 |
| Sodium Chloride | 0.1 |
| Sodium Sulfate | 0.2 |
| Limonene | 0.02 |
| Dyes, pH adjuster, Preservative | q.s. |

### Mouthwash Example Formulations

Example formulation 181: Mouthrinse without Alcohol

| Ingredient | % |
|---|---|
| Aqua | ad 100 |
| Glycerin | 11.0 |
| Propylene Glycol | 7.0 |
| Composition A | 1.0 |
| Poloxamer 407 | 0.75 |
| Aroma | 0.9 |
| Cetylpyridinium Chloride | 0.04 |
| Sodium Fluoride | 0.05 |
| Sodium Saccharin | 0.07 |
| Menthol | 0.1 |
| Dyes, pH adjuster, Preservative | q.s. |

Example formulation 182: Mouthrinse without Alcohol

| Ingredient | % |
|---|---|
| Aqua | ad 100 |
| Sorbitol | 15.0 |
| Glycerin | 7.5 |
| Composition A | 1.0 |
| Xylitol | 1.0 |
| Cellulose Gum | 0.5 |
| Zinc PCA | 0.5 |
| Zinc Hydroxyapatite | 0.05 |
| Aroma | 0.9 |
| Silica | 0.05 |
| Ricinus Communis Seed Oil | 0.1 |
| Ammonium Acryloyldimethyltaurate/VP Copolymer | 0.05 |
| Mentha Arvensis Leaf Oil | 0.01 |
| Sodium Myristoyl Sarcosinate | 0.2 |
| Sodium Methyl Cocoyl Taurate | 0.2 |
| Sodium Saccharin | 0.5 |
| Sodium Fluoride | 0.05 |
| Limonene | 0.1 |
| Dyes, pH adjuster, Preservative | q.s. |

Example formulation 183: Mouthrinse without Alcohol

| Ingredient | % |
|---|---|
| Aqua | ad 100 |
| Sorbitol | 20.0 |
| PEG-40 | 0.5 |
| Composition A | 0.5 |
| Flavor | 0.05 |
| Xanthan Gum | 0.1 |
| Zinc Sulfate | 0.15 |
| Sodium Saccharin | 0.05 |
| Sodium Fluoride | 0.05 |
| Gellan Gum | 0.2 |
| Calcium Aluminum Borosilicate | 0.1 |
| Silica | 0.05 |
| Titanium Dioxide | 0.05 |
| Tin Oxide | 0.07 |
| Eugenol | 0.15 |
| Limonene | 0.05 |
| Linalool | 0.05 |
| Dyes, pH adjuster, Preservative | q.s. |

Example formulation 184: Mouthrinse without Alcohol

| Ingredient | % |
|---|---|
| Aqua | ad 100 |
| Glycerin | 7.5 |
| Composition A | 0.5 |
| Glycolipids | 0.5 |
| Flavor | 0.3 |
| Cetylpyridinium Chloride | 0.04 |
| Sodium Saccharin | 0.05 |
| Sucralose | 0.05 |
| Sodium Fluoride | 0.02 |
| Disodium Phosphate | 0.25 |
| Dyes, pH adjuster, Preservative | q.s. |

Example formulation 185: Mouthrinse

| Ingredient | % |
|---|---|
| Aqua | ad 100 |
| Glycerin | 10.0 |
| Sorbitol | 10.0 |
| Cocamidopropyl Betaine | 1.0 |
| Composition A | 0.5 |
| Potassium Chloride | 0.1 |
| Propylene Glycol | 0.5 |
| Aroma | 0.25 |
| Sodium Fluoride | 0.02 |
| Olaflur | 0.16 |
| Potassium Acesulfame | 0.1 |
| Sodium Chloride | 0.1 |
| Limonene | 0.03 |
| Sodium Sulfate | 0.05 |
| Dyes, pH adjuster, Preservative | q.s. |

Example Example formulation 43: Mouthrinse without Alcohol

| Ingredient | % |
|---|---|
| Aqua | ad 100 |
| Sorbitol | 15.0 |
| Composition A | 1.0 |
| Aloe Barbadensis Leaf Extract | 0.15 |
| Calendula Officinalis Flower Extract | 0.2 |
| Chamomilla Recutita Flower Extract | 0.2 |
| Sodium Fluoride | 0.02 |
| Calcium Glycerophosphate | 0.05 |
| Zinc Gluconate | 0.8 |
| Mentha Spicata Crispa Herb Oil | 0.05 |
| Mentha Arvensis Leaf Oil | 0.05 |
| Menthol | 0.04 |
| Dyes, pH adjuster, Preservative | q.s. |

Example formulation 186: Mouthrinse without Alcohol

| Ingredient | % |
|---|---|
| Aqua | ad 100 |
| Glycerin | 15.0 |
| Sodium Gluconate | 1.5 |
| Composition A | 0.5 |
| PEG-40 Hydrogenated Castor Oil | 0.5 |
| Olaflur | 0.16 |
| Aroma | 0.25 |
| Stannous Chloride | 0.05 |
| Sodium Fluoride | 0.02 |
| Cocamidopropyl Betaine | 0.35 |
| Sodium Saccharin | 0.07 |
| Dyes, pH adjuster, Preservative | q.s. |

Example formulation 187: Mouthrinse without Alcohol

| Ingredient | % |
|---|---|
| Aqua | ad 100 |
| Glycerin | 10.0 |
| Sorbitol | 8.0 |
| Composition A | 1.0 |
| Glycolipids | 0.5 |
| Tetrasodium Pyrophosphate | 0.25 |
| Aroma | 0.04 |
| Limonene | 0.1 |
| Sodium Fluoride | 0.02 |
| Sodium Saccharin | 0.08 |
| Dyes, pH adjuster, Preservative | q.s. |

Example formulation 188: Mouthrinse without Alcohol

| Ingredient | % |
|---|---|
| Aqua | ad 100 |
| Glycerin | 7.5 |
| PEG-60 Hydrogenated Castor Oil | 1.0 |
| Composition A | 0.5 |
| Sodium Citrate | 0.45 |
| Aroma | 0.3 |
| Zinc Chloride | 0.15 |
| Cetylpyridinium Chloride | 0.05 |
| Sodium Saccharin | 0.08 |
| Sodium Fluoride | 0.02 |
| Dyes, pH adjuster, Preservative | q.s. |

Example formulation 189: Mouthrinse without Alcohol

| Ingredient | % |
|---|---|
| Aqua | ad 100 |
| Glycerin | 11.0 |
| Composition A | 2.0 |
| Flavor | 1.0 |
| Cetylpyridinium Chloride | 0.5 |
| Poloxamer 407 | 0.1 |
| Sodium Saccharin | 0.1 |
| Sodium Fluoride | 0.02 |
| Sucralose | 0.05 |
| Mentha Viridis Leaf Oil | 0.1 |
| Dyes, pH adjuster, Preservative | q.s. |

Example formulation 190: Mouthrinse without Alcohol

| Ingredient | % |
|---|---|
| Aqua | ad 100 |
| Glycerin | 12.0 |
| Sorbitol | 8.0 |
| Potassium Nitrate | 0.15 |
| PEG-60 Hydrogenated Castor Oil | 0.5 |
| Composition A | 0.25 |
| Poloxamer 407 | 0.25 |
| Aroma | 0.2 |
| Disodium Phosphate, Sodium Phosphate | 0.25 |
| Sodium Fluoride | 0.05 |
| Sodium Saccharin | 0.08 |
| Dyes, pH adjuster, Preservative | q.s. |

Example formulation 191: Mouthrinse without Alcohol

| Ingredient | % |
|---|---|
| Aqua | ad 100 |
| Sorbitol | 10.0 |
| PEG-40 Hydrogenated Castor Oil | 1.0 |
| Potassium Citrate | 1.0 |
| Composition A | 0.25 |
| Glycerin | 0.9 |
| Aroma | 0.4 |
| Zinc Sulfate | 0.15 |
| Sodium Saccharin | 0.1 |
| Propylene Glycol | 0.3 |
| Citrus Limon Juice | 0.05 |
| Aloe Barbadensis Leaf Extract | 0.1 |
| Limonene | 0.05 |
| Sodium Fluoride | 0.02 |
| Dyes, pH adjuster, Preservative | q.s. |

Example formulation 192: Mouthrinse without Alcohol

| Ingredient | % |
|---|---|
| Aqua | ad 100 |
| Glycerin | 12.0 |
| Composition A | 1.2 |
| Gycolipids | 1.0 |
| Hydrogenated Starch Hydrolysate | 1.0 |
| Xylitol | 1.0 |
| Steareth-30 | 0.2 |
| Amyloglucosidase | 0.5 |
| Allantoin | 0.1 |
| Glucose Oxidase | 0.5 |
| Sodium Fluoride | 0.05 |
| Limonene | 0.1 |
| Dyes, pH adjuster, Preservative | q.s. |

Example formulation 193: Mouthrinse

| Ingredient | % |
|---|---|
| Aqua | ad 100 |
| Glycerin | 11.0 |
| Propylene Glycol | 7.5 |
| Sorbitol | 7.5 |
| Tetrapotassium Pyrophosphate | 1.5 |
| Polysorbate 20 | 0.5 |
| Composition A | 0.5 |
| Tetrasodium Pyrophosphate | 0.75 |
| Zinc Citrate | 0.15 |
| PVM/MA Copolymer | 0.1 |
| Flavor | 0.2 |
| Sodium Fluoride | 0.05 |
| Sodium Saccharin | 0.07 |
| Dyes, pH adjuster, Preservative | q.s. |

Example formulation 194: Mouthrinse

| Ingredient | % |
|---|---|
| Aqua | ad 100 |
| Glycerin | 11.0 |
| Alcohol | 7.5 |
| Sorbitol | 7.5 |
| Composition A | 1.0 |
| Poloxamer 407 | 0.5 |
| Sodium Phosphate, Disodium Phosphate | 0.15 |
| Flavor | 0.9 |
| Cetylpyridinium Chloride | 0.05 |
| Sodium Saccharin | 0.08 |
| Sodium Fluoride | 0.05 |
| Dyes, pH adjuster, Preservative | q.s. |

Example formulation 195: Mouthrinse

| Ingredient | % |
|---|---|
| Aqua | ad 100 |
| Alcohols | 22.0 |
| Sorbitol | 10.0 |
| Flavor | 1.0 |
| Composition A | 1.0 |
| Sodium Saccharin | 0.09 |
| Eucalyptol | 0.1 |
| Thymol | 0.06 |
| Sucralose | 0.06 |
| Sodium Fluoride | 0.05 |
| Menthol | 0.04 |
| Dyes, pH adjuster, Preservative | q.s. |

Example formulation 196: Mouthrinse

| Ingredient | % |
|---|---|
| Aqua | ad 100 |
| Glycerin | 12.0 |
| Xylitol | 7.0 |
| Disodium Phosphate | 1.5 |
| Composition A | 1.0 |
| Cocamidopropyl Betaine | 0.5 |
| Propylene Glycol | 0.5 |
| Stannous Fluoride | 0.09 |
| Aroma | 0.3 |
| Mentha Piperita Leaf Extract | 0.1 |
| Chamomilla Recutita Flower Extract | 0.2 |
| Salvia Officinalis Leaf Extract | 0.25 |
| Commiphora Myrrha Resin Extract | 0.15 |
| Sodium Saccharin | 0.1 |
| Citral | 0.05 |
| Limonene | 0.02 |
| Linalool | 0.02 |
| Dyes, pH adjuster, Preservative | q.s. |

Example formulation 55: Mouthrinse

| Ingredient | % |
|---|---|
| Aqua | ad 100 |
| Glycerin | 11.0 |
| Sorbitol | 11.0 |
| Propylene Glycol | 3.0 |
| Disodium Pyrophosphate | 1.5 |
| PEG-40 Hydrogenated Castor Oil | 1.0 |
| Composition A | 0.5 |
| PVM/MA Copolymer | 0.5 |
| Tetrapotassium Pyrophosphate | 0.25 |
| Sodium Levulinate | 0.25 |
| Sodium Anisate | 0.25 |
| Olaflur | 0.16 |
| Aroma | 0.8 |
| Arginine | 0.8 |
| Sodium Saccharin | 0.07 |
| Sodium Fluoride | 0.02 |
| Dyes, pH adjuster, Preservative | q.s. |

Example formulation 197: Mouthrinse

| Ingredient | % |
|---|---|
| Aqua | ad 100 |
| Alcohol Denat. | 33.0 |
| Glycerin | 10.0 |
| Sorbitol | 8.0 |
| PEG-40 Hydrogenated Castor Oil | 1.0 |
| Composition A | 1.0 |
| Glycolipids | 0.5 |
| Aroma | 0.6 |
| Menthol | 0.05 |
| Sodium Saccharin | 0.07 |
| Aluminum Lactate | 0.2 |
| Thymol | 0.06 |
| Curcuma Xanthorrhiza Root Extract | 0.05 |
| Sodium Fluoride | 0.02 |
| Dyes, pH adjuster, Preservative | q.s. |

Example formulation 198: Mouthrinse

| Ingredient | % |
|---|---|
| Aqua | ad 100 |
| Sorbitol | 12.5 |
| Propylene Glycol | 3.5 |
| Composition A | 0.5 |
| Sodium Lauryl Sulfate | 0.5 |
| Poloxamer 407 | 0.5 |
| Flavor | 0.3 |
| Eucalyptol | 0.08 |
| Methyl Salicylate | 0.1 |
| Thymol | 0.05 |
| Stannous Fluoride | 0.09 |
| Menthol | 0.1 |
| Sodium Saccharin | 0.06 |
| Sucralose | 0.7 |
| Camellia Sinensis Leaf Extract | 0.05 |
| Dyes, pH adjuster, Preservative | q.s. |

Example formulation 199: Mouthrinse

| Ingredient | % |
|---|---|
| Aqua | ad 100 |
| Composition A | 1.0 |
| Eucalyptol | 0.9 |
| Zinc Chloride | 0.25 |
| Methyl Salicylate | 0.05 |
| Thymol | 0.05 |
| Levomenthol | 0.07 |
| Stannous Fluoride | 0.09 |
| Dyes, pH adjuster, Preservative | q.s. |

Example formulation 200: Mouthrinse

| Ingredient | % |
|---|---|
| Aqua | ad 100 |
| Alcohol Denat. | 15.0 |
| Sorbitol | 5.0 |
| Glycine | 1.0 |
| Composition A | 1.0 |
| Aroma | 0.2 |
| Zinc Sulfate | 0.2 |
| Sodium Fluoride | 0.02 |
| Sodium Saccharin | 0.07 |
| Dyes, pH adjuster, Preservative | q.s. |

Example formulation 201: Mouthrinse

| Ingredient | % |
|---|---|
| Aqua | ad 100 |
| Sorbitol | 11.0 |
| Alcohol Denat. | 11.0 |
| Xylitol | 5.0 |
| PEG-40 Hydrogenated Castor Oil | 0.75 |
| Composition A | 0.5 |
| Isopropyl Alcohol | 0.5 |
| Menthol | 0.05 |
| Sodium Citrate | 0.15 |
| Mentha Piperita Herb Oil | 0.25 |
| Sodium Saccharin | 0.08 |
| Methyl Diisopropyl Propionamide | 0.05 |
| Thymol | 0.07 |
| Glycolipids | 0.1 |
| Sodium Fluoride | 0.05 |
| O-cymen-5-ol | 0.05 |
| Cyclodextrin | 0.07 |
| Eugenia Caryophyllus Leaf Oil | 0.1 |
| Propylene Glycol | 0.25 |
| Butylene Glycol | 0.25 |
| Psidium Guajava Leaf Extract | 0.08 |
| Phyllanthus Emblica Fruit Extract | 0.08 |
| Echinacea Purpurea Root Extract | 0.08 |
| Salvia Officinalis Leaf Extract | 0.2 |
| Dyes, pH adjuster, Preservative | q.s. |

Example formulation 202: Mouthrinse

| Ingredient | % |
|---|---|
| Aqua | ad 100 |
| Alcohols | 15.0 |
| Sorbitol | 7.0 |
| Sodium Lauroyl Sarcosinate | 1.0 |
| Composition A | 0.25 |
| PEG-40 Hydrogenated Castor Oil | 0.25 |
| Aroma | 0.4 |
| Cetylpyridinium Chloride | 0.04 |
| Sodium Citrate | 0.15 |
| Sodium Saccharin | 0.08 |
| Sodium Fluoride | 0.05 |
| Limonene | 0.05 |
| Dyes, pH adjuster, Preservative | q.s. |

Example formulation 203: Mouthrinse

| Ingredient | % |
|---|---|
| Aqua | ad 100 |
| Alcohol denat. | 18.0 |
| Sorbitol | 8.0 |
| Aroma | 1.0 |
| Poloxamer 407 | 0.9 |
| Methyl salicylate | 0.2 |
| Sodium Saccharin | 0.2 |
| Sodium Fluoride | 0.05 |
| Composition A | 0.9 |
| Chlorhexidine Digluconate | 0.2 |
| PEG-40 Hydrogenated Castor Oil | 0.2 |
| Dyes, pH adjuster, Preservative | q.s. |

Example formulation 204: Mouthrinse

| Ingredient | % |
|---|---|
| Aqua | ad 100 |
| Glycerin | 10.0 |
| Hydrogenated Starch Hydrolysate | 1.0 |
| PVP | 0.5 |
| Composition A | 0.5 |
| Aroma | 0.4 |
| Zinc Acetate | 0.2 |
| Sodium Fluoride | 0.32 |
| Potassium Acesulfame | 0.15 |
| Chlorhexidine Digluconate | 0.2 |
| Dyes, pH adjuster, Preservative | q.s. |

Example formulation 205: Mouthrinse

| Ingredient | % |
|---|---|
| Aqua | ad 100 |
| Glycerin | 12.0 |
| Sorbitol | 7.0 |
| Composition A | 0.75 |
| PEG-40 Hydrogenated Castor Oil | 0.5 |
| Chlorhexidine Gluconate | 0.2 |
| Mentha Piperita Oil | 0.2 |
| Sodium Fluoride | 0.05 |
| Sodium Saccharin | 0.2 |
| Dyes, pH adjuster, Preservative | q.s. |

Example formulation 206: Mouthrinse

| Ingredient | % |
|---|---|
| Aqua | ad 100 |
| Glycerin | 15.0 |
| Macrogolglycerol Hydroxystearate | 7.0 |
| Sorbitol | 7.0 |
| Composition A | 0.5 |
| Cocamidopropyl Betaine | 0.2 |
| Sodium Fluoride | 0.02 |
| Chlorhexidine Digluconate | 0.2 |
| Flavor | 0.5 |
| Dyes, pH adjuster, Preservative | q.s. |

Example formulation 207: Mouthrinse Concentrate

| Ingredient | % |
|---|---|
| Propylene Glycol | 55.0 |
| Aqua | ad 100 |
| Aroma | 2.0 |
| Sodium Saccharin | 0.4 |
| Composition A | 0.75 |
| Polysorbate 20 | 0.25 |
| Commiphora Myrrha Oil | 2.0 |
| Salvia Officinalis Oil | 0.2 |
| Mentha Piperita Oil | 0.2 |
| Chamomilla Recutita Flower Extract | 0.2 |
| Limonene | 0.1 |
| Sodium Fluoride | 0.02 |
| Dyes, pH adjuster, Preservative | q.s. |

Example formulation 208: Mouthrinse Concentrate

| Ingredient | % |
|---|---|
| Propylene Glycol | 45.0 |
| PEG-6 | 5.0 |
| Aqua | ad 100 |
| Composition A | 1.0 |
| Bisabolol | 0.2 |
| Zinc Chloride | 0.25 |
| Cetylpyridinium Chloride | 0.1 |
| Sodium Saccharin | 0.5 |
| Sodium Fluoride | 0.05 |
| Salvia Officinalis Oil | 2.5 |
| Eugenol | 0.2 |
| Limonene | 0.1 |
| Linalool | 0.1 |
| Citronellol | 0.1 |
| Dyes, pH adjuster, Preservative | q.s. |

Example formulation 209: Mouthrinse Concentrate

| Ingredient | % |
|---|---|
| Alcohols | 55.0 |
| Aqua | ad 100 |
| Aroma | 1.5 |
| Composition A | 0.7 |
| Sodium C14-17 Alkyl Sulfonate | 0.7 |
| Mentha Arvensis Leaf Oil | 2.0 |
| Sodium Saccharin | 0.5 |
| Chamomilla Recutita Flower Extract | 0.5 |
| Commiphora Myrrha Resin Extract | 0.2 |
| Salvia Officinalis Leaf Extract | 0.2 |
| Sodium Fluoride | 0.02 |
| Limonene | 0.1 |
| Dyes, pH adjuster, Preservative | q.s. |

Example formulation 210: Peroxide Mouthrinse

| Ingredient | % |
|---|---|
| Aqua | ad 100 |
| Glycerin | 11.0 |
| Alcohol | 5.0 |
| H₂O₂ 35% | 4.30 |
| Polyphosphate | 1.0 |
| Menthol | 0.05 |
| Coolant | 0.02 |
| Flavor | 0.15 |
| Calcium Chloride | 0.03 |
| Composition A | 0.50 |
| Sodium Saccharin | 0.1 |
| Sodium Fluoride | 0.22 |
| Dyes, pH adjuster, Preservative | q.s. |

Example formulation 211: Fluoride Gel

| Ingredient | % |
|---|---|
| Aqua | ad 100 |
| Propylene Glycol | 30.0 |
| Hydroxyethylcellulose | 3.0 |
| Saccharin | 1.0 |
| Composition A | 0.5 |
| Pyrus Malus Fruit | 0.2 |
| Mentha Piperita Oil | 0.2 |
| Flavor | 0.5 |
| Aminofluoride Dectafluor | 0.29 |
| Olaflur | 3.03 |
| Sodium Fluoride | 2.21 |
| Dyes, pH adjuster, Preservative | q.s. |

Example formulation 212: Fluoride Gel

| Ingredient | % |
|---|---|
| Aqua | ad 100 |
| Sorbitol | 15.00 |
| Silica | 5.0 |
| PEG 600 | 3.5 |
| Tetrapotassium Pyrophosphate | 1.5 |
| Xanthan Gum | 0.5 |
| Sodium Lauryl Sulfate | 0.75 |
| Composition A | 0.75 |
| Aroma | 0.9 |
| Methol | 0.05 |
| Anethol | 0.05 |
| Citronellol | 0.2 |
| Sodium Fluoride | 5.0 |
| Dyes, pH adjuster, Preservative | q.s. |

Example formulation 213: Dental Floss

| Ingredient | % |
|---|---|
| PTFE | ad 100 |
| Aroma | 1.0 |
| Acacia Senegal Gum | 0.7 |
| Cera Alba | 0.7 |
| Potassium Acesulfame | 0.5 |
| Sodium Lauryl Sulfate | 0.25 |
| Composition A | 0.25 |
| Glycerin | 0.7 |
| Silica | 0.5 |
| Sodium Fluoride | 0.22 |

Example formulation 214: Teeth Wipe

| Ingredient | % |
|---|---|
| Aqua | q.s. 100 |
| Alcohol Denat. | 30.00 |
| Glycerin | 20.00 |
| Stevia | 1.0 |
| Polysorbate 20 | 0.5 |
| Composition A | 0.5 |
| Aroma | 1.0 |
| Dyes, pH adjuster, Preservatives | q.s. |

Example formulation 215: Chewing Gum

| Ingredient | % |
|---|---|
| Gum Base | ad 100 |
| Sorbit | 40.0 |
| Isomalt | 9.0 |
| Xylitol | 2.5 |
| Mannit D | 2.5 |
| Aspartame | 0.1 |
| Acesulfam K | 0.05 |
| Emulgum | 0.2 |
| Sorbitol | 12.00 |
| Glycerin | 1.0 |
| Composition A | 1.0 |
| Flavor | 1.0 |
| Sodium Fluoride | 0.05 |
| Dyes, pH adjuster, Preservative | q.s. |

Example formulation 216: Mouthwash Tabs

| Ingredient | % |
|---|---|
| Dicalcium Phosphate Anhydrous | ad 100 |
| Sodium Bicarbonate | 35.0 |
| Silica | 7.0 |
| Citric Acid | 4.0 |
| Composition A | 3.0 |
| Sorbitol | 1.0 |
| Glycerin | 1.0 |
| Stevia Rebaudiana Extract (Stevia) | 0.2 |
| Xylitol | 0.5 |
| Aroma | 0.1 |
| Mentha Piperita Oil | 0.1 |
| Dyes, pH adjuster, Preservative | q.s. |

Example formulation 217: Tooth Tab

| Ingredient | % |
|---|---|
| Dicalcium Phosphate Anhydrous | ad 100 |
| Potassium Bitartrate | 15.0 |
| Sorbitol | 15.0 |
| Composition A | 6.0 |
| Lauroyl Sarcosine | 0.8 |
| Kaolin | 0.5 |
| Sodium Saccharin | 0.5 |
| Aroma | 0.2 |
| Dyes, pH adjuster, Preservative | q.s. |

Example formulation 218: Powder Toothpaste

| Ingredient | % |
|---|---|
| Calcium Carbonate | ad 100 |
| Sorbitol | 15.0 |
| Kaolin | 15.0 |
| Sodium Bicarbonate | 15.0 |
| Composition A | 1.5 |
| Aroma | 1.0 |
| Citrus Limon Peel Oil | 1.0 |
| Stevia Rebaudiana Extract | 0.5 |
| Xylitol | 0.5 |
| Citral | 0.1 |
| Geraniol | 0.1 |
| Limonene | 0.1 |
| Dyes, pH adjuster, Preservative | q.s. |

Further example formulations (in wt.-%):

| | I | II | III | IV | V | VI | VII | VIII | IX | X |
|---|---|---|---|---|---|---|---|---|---|---|
| Water | ad 100 | | | | | | | | | |
| Composition A | 9.0 | 15.0 | 5.5 | 5.0 | 7.0 | 3.0 | 6.0 | 4.0 | 3.5 | 3.0 |
| Sodium Laureth Sulfate | — | — | — | 7.0 | — | — | — | 4.5 | — | — |
| Sodium Lauryl Sulfate | — | — | — | — | 5.0 | — | — | — | 3.5 | 1.0 |
| Cocamidopropyl Betaine | — | — | — | — | — | 6.0 | — | 3.0 | 2.0 | 4.5 |
| Sodium Cocoamphoacetate | — | — | — | — | — | — | 2.0 | — | 3.5 | — |
| Coco-Glucoside | — | — | — | — | — | — | 5.5 | 1.5 | — | — |
| Sodium Cocoyl Glutamate | — | — | — | — | — | — | 0.7 | — | — | — |
| Stearic Acid | — | — | — | — | — | — | — | 1.0 | — | 3.5 |
| Glyceryl Glucoside | — | 0.5 | — | — | 0.3 | — | 0.2 | — | — | — |
| Sucrose Cocoate | 0.5 | — | 1.0 | 1.0 | 1.0 | 0.5 | — | 1.0 | 1.0 | 1.0 |
| Glycerin | — | 1.0 | 0.5 | — | — | 0.3 | 1.5 | 1.0 | — | 1.0 |
| PEG-7 Glyceryl Cocoate | — | 0.7 | — | 0.5 | — | — | — | — | — | 0.5 |
| Trideceth-9 | — | — | — | 0.2 | — | — | — | 0.3 | — | — |
| Polyglyceryl-4 Caprate | — | — | 1.0 | — | 0.4 | — | 0.5 | — | — | — |
| Polyquaternium-10 | — | 0.2 | — | 0.1 | — | — | — | 0.2 | — | — |
| Hydroxypropyl Guar | 0.2 | — | 0.3 | 0.2 | 0.2 | 0.2 | 0.3 | 0.1 | 0.2 | — |
| Hydroxypropyltrimonium | | | | | | | | | | |
| Chloride | | | | | | | | | | |
| Silicone Quaternium-22 | — | — | — | 0.5 | — | — | — | 0.5 | — | — |
| Dimethicone | — | 0.5 | — | — | — | — | — | 1.0 | — | — |
| Amodimethicone | — | 0.1 | — | — | 1.0 | 0.3 | — | — | 0.5 | — |
| Argania Spinosa Oil | — | — | 0.2 | 0.1 | — | 0.1 | 0.2 | — | — | — |
| Glycol Distearate | 0.5 | 0.5 | — | — | 0.5 | — | 0.3 | 0.5 | 0.5 | 0.5 |
| Acrylates/C10-30 Alkyl | 1.0 | — | — | — | 0.5 | — | — | — | 0.5 | — |
| Acrylate Crosspolymer | | | | | | | | | | |
| Sodium Hydroxide, 25% | 1.5 | — | — | — | 0.8 | — | — | — | 0.7 | — |
| Isostearamide MIPA; | — | — | — | — | — | 2.5 | — | — | 1.0 | 0.5 |
| Glyceryl Laurate | | | | | | | | | | |
| Cocamide DEA | — | — | — | 1.0 | 0.5 | — | — | — | — | — |
| Sodium Chloride | 0.1 | 1.2 | — | 0.5 | — | 1.5 | — | 2.0 | — | 1.2 |
| PEG-120 Methyl Glucose | — | 3.5 | — | 0.2 | — | — | — | 0.6 | — | — |
| Dioleate | | | | | | | | | | |
| Xanthan Gum | — | — | 1.2 | — | — | 0.2 | 1.5 | — | — | — |
| Cellulose | — | — | — | 0.1 | — | 0.1 | 0.1 | — | 0.1 | — |
| Zinc Pyrithione | — | 0.1 | — | 1.1 | — | — | — | 0.5 | — | — |
| Benzophenone-4 | — | 0.1 | — | 0.2 | — | — | — | 0.2 | 0.1 | — |
| Tetrasodium EDTA | 0.1 | 0.1 | — | 0.1 | — | — | — | 0.1 | 0.1 | — |
| Caffeine | — | — | 0.1 | — | — | — | — | 0.1 | 0.1 | — |
| Hydrolyzed Keratin | — | — | 0.1 | 0.1 | — | 0.1 | 0.2 | — | 0.1 | — |
| Panthenol | 0.1 | 0.1 | 0.1 | 0.1 | 0.1 | 0.1 | — | 0.1 | 0.1 | 0.1 |

| | |
|---|---|
| Citric Acid | ad pH |
| Parfums, Dyes, | q.s. |
| Preservatives | |

| | XI | XII | XIII | XIV | XV | XVI | XVII | XVIII | XIX | XX |
|---|---|---|---|---|---|---|---|---|---|---|
| Water | ad 100 | | | | | | | | | |
| Composition A | 10.0 | 7.0 | 5.0 | 4.0 | 7.0 | 2.5 | 6.0 | 4.0 | 2.0 | 1.0 |
| Sodium Laureth Sulfate | — | — | 8.0 | — | — | — | — | 3.5 | — | — |
| Coco-Betaine | — | — | — | 5.5 | 5.0 | — | — | 3.0 | — | — |
| Cocamidopropyl Betaine | — | — | — | — | — | 6.0 | — | — | 3.0 | 4.0 |
| Sodium Cocoamphoacetate | — | — | — | — | 2.0 | — | — | — | — | — |
| Coco Glucoside | — | — | — | — | 2.0 | — | 4.0 | 1.5 | 6.0 | — |
| Glycolipids | — | — | — | — | — | — | 2.5 | 0.5 | 2.5 | 0.3 |
| Stearic Acid | — | 8.0 | — | — | — | — | — | — | — | 4.5 |
| Sodium Cocoyl Glycinate | — | — | — | — | — | 3.0 | 0.5 | — | — | 0.3 |
| Sodium Lauroyl Methyl | — | — | — | — | — | 1.0 | — | 1.0 | 2.0 | 0.5 |
| Isethionate | | | | | | | | | | |
| Sucrose Cocoate | 0.5 | 1.1 | — | 1.0 | 1.0 | — | — | 0.3 | 1.0 | 0.3 |
| Glycerin | 1.5 | 0.3 | 0.5 | — | 0.3 | 0.5 | 1.0 | 0.5 | — | 1.0 |
| PEG-7 Glyceryl Cocoate | — | — | 0.5 | — | — | 0.8 | — | 0.5 | — | 0.5 |
| PEG-40 Hydrogenated | — | — | 0.5 | — | — | 1.5 | — | 0.3 | — | — |
| Castor Oil | | | | | | | | | | |
| Polyglyceryl-4 Caprate | 0.5 | — | — | — | 0.4 | — | 0.5 | — | 1.1 | — |
| Polyquaternium-11 | — | 0.2 | — | — | 0.1 | 0.3 | — | 0.2 | — | — |
| Guar | 0.3 | — | 0.3 | 0.2 | 0.2 | — | 0.3 | 0.1 | 0.4 | 0.2 |
| Hydroxypropyltrimonium | | | | | | | | | | |
| Chloride | | | | | | | | | | |
| Silicone Quaternium-22 | — | — | 0.5 | — | — | — | — | 1.5 | — | — |
| PEG-12 Dimethicone | — | — | — | — | — | 1.5 | — | — | — | — |
| Dimethicone | — | — | 1.0 | — | 0.5 | — | — | — | — | — |
| Aminopropyl Dimethicone | — | — | — | — | 0.3 | — | — | — | — | — |
| Helianthus Annuus Oil | 0.1 | — | 0.2 | 0.1 | — | 0.1 | 0.3 | 0.2 | — | 0.1 |
| PEG-3 Distearate | — | — | 0.5 | — | — | 0.8 | — | 0.5 | — | — |
| Acrylates/C10-30 Alkyl | — | — | 0.2 | — | 0.5 | — | — | — | — | — |
| Acrylate Crosspolymer | | | | | | | | | | |
| Sodium Hydroxide, 25% | — | — | 0.3 | — | 0.8 | — | — | — | — | — |
| Cocamide DEA | — | — | — | — | — | 2.5 | — | — | — | 0.5 |
| Cocamide MEA | — | — | — | 1.0 | — | | — | 1.0 | — | — |
| Sodium Chloride | 0.4 | 0.7 | 0.1 | — | — | 1.5 | — | 2.0 | — | 1.2 |
| Propylene Glycol (and) | — | 2.5 | 2.0 | — | — | 0.3 | — | 0.6 | — | 0.2 |
| PEG-55 Propylene Glycol | | | | | | | | | | |
| Oleate | | | | | | | | | | |
| Xanthan Gum | 1.2 | — | — | 1.0 | 0.2 | — | 1.5 | 0.1 | 1.1 | — |
| Hydroxyethyl Cellulose | 0.1 | — | — | 0.1 | — | 0.1 | 0.1 | — | — | — |
| Benzophenone-4 | — | 0.1 | 0.2 | — | — | 0.2 | — | 0.2 | — | 0.1 |
| Menthol | 0.1 | — | — | — | — | — | — | — | — | 0.1 |
| Tetrasodium Glutamate | — | 0.1 | — | 0.1 | 0.1 | — | 0.1 | 0.1 | — | 0.1 |
| Diacetate | | | | | | | | | | |
| Caffeine | 0.1 | — | 0.1 | — | — | — | — | 0.1 | — | — |
| Coumarin | 0.1 | — | — | 0.1 | 0.1 | — | 0.1 | 0.1 | — | 0.1 |
| Hydrolyzed Wheat Protein | 0.1 | — | 0.1 | 0.1 | — | — | 0.2 | 0.1 | 0.1 | 0.1 |
| Panthenol | 0.1 | 0.1 | — | 0.1 | 0.1 | 0.1 | 0.1 | — | 0.1 | 0.1 |

| Citric Acid | ad pH | | | | | | | | | |
|---|---|---|---|---|---|---|---|---|---|---|
| Parfums, Dyes, | q.s. | | | | | | | | | |
| Preservatives | | | | | | | | | | |
| | XXI | XXII | XXIII | XIV | XXV | XXVI | XXVII | XXVIII | XXIX | XXX |
| Water | ad 100 | | | | | | | | | |
| Composition A | 6.0 | 4.0 | 3.5 | 1.0 | 3.0 | 3.0 | 4.0 | 2.0 | 2.5 | 5.0 |
| Sodium Coco-Sulfate | 3.0 | 1.0 | — | 5.0 | 3.0 | 3.0 | 2.0 | — | 2.5 | 3.0 |
| Glycolipids | 1.0 | | 1.5 | 1.0 | | 2.0 | | — | 2.0 | — |
| Lauryl Glucoside | — | 2.0 | — | — | | — | — | 2.0 | 3.5 | 2.0 |
| Sodium Cocoyl Glutamate | 1.0 | — | — | — | 3.0 | — | — | — | | — |
| Sodium Cocoyl Glycinate | — | — | 1.5 | — | — | — | — | 2.5 | 1.5 | — |
| Sodium C14-16 Olefin | | | | | 3.0 | | 2.0 | — | — | 2.0 |
| Sulfonate | | | | | | | | | | |
| Lauroyl/Myristoyl Methyl | — | 1.0 | — | 2.0 | — | | — | — | — | — |
| Glucamide | | | | | | | | | | |
| Cocamidopropyl Betaine | — | 2.0 | 1.5 | 4.0 | — | 2.0 | — | 7.5 | — | — |
| Sodium Cocoamphoacetate | — | 2.0 | 5.0 | — | 2.0 | 2.0 | 4.0 | — | — | — |
| Sucrose Cocoate | 1.0 | — | 1.5 | — | 1.5 | 1.0 | — | 1.0 | — | 3.5 |
| Glycerin | 0.5 | 0.5 | — | — | 0.3 | — | 0.2 | — | 0.5 | — |
| Alcohol Denat. | — | — | 3.0 | — | — | 2.0 | — | — | — | — |
| Sorbitan Sesquicaprylate | 1.0 | — | 0.7 | 1.0 | | 0.5 | — | 0.5 | — | 1.0 |
| Isostearamide MIPA; | — | 1.0 | — | — | — | 2.0 | — | — | — | 0.5 |
| Glyceryl Laurate | | | | | | | | | | |
| Xanthan Gum | 1.0 | 0.7 | 0.5 | — | 0.7 | 0.3 | 0.7 | 0.5 | — | 0.5 |
| Chondrus Crispus | — | — | 1.5 | | 2.0 | 1.5 | 0.7 | — | — | 0.5 |
| Carrageenan | — | — | — | 1.0 | — | — | — | 0.3 | — | 0.5 |
| Hydroxypropyl Starch | — | — | — | — | — | — | — | 4.5 | 5.5 | — |
| Phosphate | | | | | | | | | | |
| Guar | 0.2 | — | 0.5 | 0.2 | 0.2 | 0.5 | 0.3 | 0.1 | — | — |
| Hydroxpropyltrimonium | | | | | | | | | | |
| Chloride | | | | | | | | | | |
| Bis-(Isostearoyl/Oleyl | 0.5 | 0.5 | — | — | 0.5 | — | — | — | 0.5 | — |
| Isopropyl) Dimonium | | | | | | | | | | |
| Methosulfate | | | | | | | | | | |
| Prunus Amygdalus Dulcis | — | 0.1 | — | 0.1 | — | 0.05 | — | — | — | — |
| (Almond) Oil | | | | | | | | | | |
| Lemon Oil | | — | 0.2 | — | 0.1 | | 0.2 | | | |
| Cellulose | 0.5 | — | — | — | 0.7 | — | 0.3 | 0.1 | — | 0.5 |
| Charcoal Powder | 1.0 | — | — | — | — | — | — | — | — | — |
| Saccharum Officinarum | — | — | — | 0.1 | — | 0.2 | — | — | — | — |
| (Sugar Cane) Extract | | | | | | | | | | |
| Sodium Chloride | 0.7 | — | — | 0.5 | 1.0 | 0.7 | — | 2.0 | — | 1.2 |
| Polyglyceryl-6 Caprylate; | | 3.0 | — | 4.0 | | 2.5 | — | | | |
| Polyglyceryl-3 Cocoate; | | | | | | | | | | |
| Polyglyceryl-4 Caprate; | | | | | | | | | | |
| Polyglyceryl-6 Ricinoleate | | | | | | | | | | |
| Caprylyl/Capryl Glucoside; | | — | 3.0 | — | 1.0 | | 2.0 | | | |
| Aqua, Sodium Cocoyl | | | | | | | | | | |
| Glutamate; Glyceryl | | | | | | | | | | |
| Caprylate; Ccitric Acid; | | | | | | | | | | |
| Polyglyceryl-6 Oleate, | | | | | | | | | | |
| Sodium Surfactin | | | | | | | | | | |
| Tocopherol | — | 0.1 | — | 0.1 | — | 0.1 | 0.1 | — | 0.1 | — |
| Phytic Acid | 0.1 | 0.1 | — | — | — | — | — | 0.1 | 0.1 | — |

| Citric Acid | ad pH | | | | | | | |
|---|---|---|---|---|---|---|---|---|
| Parfums, Dyes, | q.s. | | | | | | | |
| Preservatives | | | | | | | | |
| | XXXI | XXXII | XXXIII | XXXIV | XXXV | XXXVI | XXXVII | XXXVIII |

| | |
|---|---|
| Water | ad 100 |

| | | | | | | | | |
|---|---|---|---|---|---|---|---|---|
| Composition A | 1.0 | 1.5 | 2.0 | 1.5 | 3.0 | 2.5 | 4.0 | 5.0 |
| Capryl/Capramidopropyl | — | 2.0 | — | — | 1.0 | — | 2.0 | |
| Betaine | | | | | | | | |
| Glycolipids | 1.0 | — | — | — | 1.0 | — | — | |
| Decyl Glucoside | 1.0 | — | — | — | — | 2.0 | — | |
| Disodium | — | — | 3.0 | 2.0 | — | — | 2.0 | |
| Cocoamphodiacetate | | | | | | | | |
| PEG-6 Caprylic/Capric | 3.0 | 1.5 | — | — | — | — | — | 2.0 |
| Glycerides | | | | | | | | |
| Polyglyceryl-6 Caprylate; | | — | 2.0 | — | — | 3.5 | — | |
| Polyglyceryl-4 Caprate | | | | | | | | |
| Polyglyceryl-6 Caprylate; | — | 3.0 | | — | 4.0 | — | — | |
| Polyglyceryl-3 Cocoate; | | | | | | | | |
| Polyglyceryl-4 Caprate; | | | | | | | | |
| Polyglyceryl-6 Ricinoleate | | | | | | | | |
| Polyglyceryl-4 Caprate | — | — | | 2.5 | | — | — | |
| Persea Gratissima | — | 0.1 | | — | 0.2 | — | 0.1 | 0.05 |
| (Avocado) Oil | | | | | | | | |
| Rosemary Oil | 0.1 | — | 0.05 | — | 0.1 | — | — | |
| Hydroxypropyl | — | 0.5 | | — | — | — | 0.5 | |
| Methylcellulose | | | | | | | | |
| Sorbitan Sesquicaprylate | 0.3 | — | | 0.5 | — | 0.3 | 0.5 | 0.1 |
| Betaine | — | 0.5 | | 1.0 | — | 1.0 | — | 1.0 |
| Allantoin | — | — | 0.1 | — | 0.1 | | — | |
| Starch | — | — | — | 0.1 | 0.05 | | — | 0.1 |
| Glycerin | 3.0 | 1.0 | — | — | — | 1.0 | 1.0 | — |
| Hexylene Glycol | 1.0 | — | 1.0 | — | 1.0 | 1.0 | — | — |
| Methylpropanediol; | 1.2 | — | — | — | — | — | — | — |
| Caprylyl Glycol; | | | | | | | | |
| Benzoic Acid | | | | | | | | |
| Benzyl Alcohol, Caprylyl | — | 1.0 | 1.0 | 1.0 | 1.0 | — | — | — |
| Glycol, Benzoic Acid | | | | | | | | |
| Aqua, Sodium | — | — | — | — | — | 1.0 | 1.0 | 1. |
| Levulinate, Sodium | | | | | | | | |
| Benzoate | | | | | | | | |

| | |
|---|---|
| pH adjuster (Citric Acid | ad pH |
| or NaOH) | |
| Preservatives, Parfum | q.s. |

1. A composition comprising:
a) at least one sophorolipid
b) lactic acid and/or a salt thereof,
wherein:
a) is comprised in an amount of from 1.2 wt.-% to 70.0 wt.-% and
b) is comprised in an amount of from 0.08 wt.-% to 10.0 wt.-%.

2. composition according to claim 1, wherein a total content of monohydric alcohols is selected from the group consisting of: methanol, ethanol, propanol, isopropanol and butanol.

3. The composition according to claim 1, further comprising water.

4. The composition according to claim 1, wherein:
a weight ratio of a) to b) is from 5:1 to 30:1.

5. The composition according to claim 1, wherein:
the component a) has a content of lactone type sophorolipids of more than 50 wt.-%, of the total component a).

6. The composition according to claim 1, wherein:
the component a) has a content of acid type sophorolipids of from 55 wt.-% to 99 wt.-%, of the total component a).

7. The composition according to claim 1, wherein:
in component b), L-enantiomer content is higher than 51.0 wt.-%, of the total component b).

8. The composition according to claim 1, wherein the composition has a pH of 3.5 to 11.0.

9. The composition according to claim 1 wherein:
a) is comprised in an amount of from 30.0 wt.-% to 55.0 wt.-%,
b) is comprised in an amount of from 1.0 wt.-% to 5.0 wt.-%, and
water is comprised in an amount of from 30.0 wt.-% to 69.0 wt.-%,
the weight percentages refer to the sum of a), b) and water.

10. The composition according to claim 1, wherein:
a) is comprised in an amount of from 30.0 wt.-% to 55.0 wt.-%,
b) is comprised in an amount of from 1.0 wt.-% to 5.0 wt.-%, and
water is comprised in an amount of from 40.0 wt.-% to 69.0 wt.-%,
the weight percentages refer to the total composition.

11. The composition according to claim 1, wherein:
a) is comprised in an amount of from 1.2 wt.-% to 10.0 wt.-%, and
b) is comprised in an amount of from 0.08 wt.-% to 3.0 wt.-%,
the weight percentages refer to the total composition.

12. The composition according to claim 1, further comprising a biosurfactant.

13. A method comprising:
utilizing a composition according to claim 1 in cleaning a surface, optionally wherein the surface is at least one selected from the group consisting of skin, a dish surface, and a textile's surface.

14. A method comprising:
utilizing a composition according to claim 1 in moisturizing human skin.

15. The composition according to claim 2, wherein the total content of the monohydric alcohols is in a range of from 0.0 wt.-% to 10.0 wt.-% of the total composition.

16. The composition according to claim 3, wherein the water is in an amount of from 25.0 wt.-% to 95.0 wt.-% of the total composition.

17. The composition according to claim 12, wherein the biosurfactant is at least one selected from the group consisting of rhamnolipids, glucolipids, cellulose lipids, mannosylerythritol lipids, and trehalose lipids.
